SEQUENCE LISTING (844 sequences)

SEQ ID NO: 1 - protein, 7 aa, Artificial Sequence
  Description of Artificial Sequence Synthetic peptide
RREHLVR

SEQ ID NO: 2 - protein, 7 aa, Artificial Sequence
  Description of Artificial Sequence Synthetic peptide
QSAHLKR

SEQ ID NO: 3 - protein, 7 aa, Artificial Sequence
  Description of Artificial Sequence Synthetic peptide
LKEHLTR

SEQ ID NO: 4 - protein, 7 aa, Artificial Sequence
  Description of Artificial Sequence Synthetic peptide
EAHHLSR

SEQ ID NO: 5 - protein, 7 aa, Artificial Sequence
  Description of Artificial Sequence Synthetic peptide
RQDNLGR

SEQ ID NO: 6 - protein, 7 aa, Artificial Sequence
  Description of Artificial Sequence Synthetic peptide
QQTNLTR

SEQ ID NO: 7 - protein, 7 aa, Artificial Sequence
  Description of Artificial Sequence Synthetic peptide
DRGNLTR

SEQ ID NO: 8 - protein, 7 aa, Artificial Sequence
  Description of Artificial Sequence Synthetic peptide
VRHNLTR

SEQ ID NO: 9 - protein, 7 aa, Artificial Sequence
  Description of Artificial Sequence Synthetic peptide
RTDTLAR

SEQ ID NO: 10 - protein, 7 aa, Artificial Sequence
  Description of Artificial Sequence Synthetic peptide
QSTTLKR

SEQ ID NO: 11 - protein, 7 aa, Artificial Sequence
  Description of Artificial Sequence Synthetic peptide
DSSVLRR

SEQ ID NO: 12 - protein, 7 aa, Artificial Sequence
  Description of Artificial Sequence Synthetic peptide
QRSDLTR

SEQ ID NO: 13 - protein, 7 aa, Artificial Sequence
  Description of Artificial Sequence Synthetic peptide
RREVLEN

SEQ ID NO: 14 - protein, 7 aa, Artificial Sequence
  Description of Artificial Sequence Synthetic peptide
QRSSLVR

SEQ ID NO: 15 - protein, 7 aa, Artificial Sequence
  Description of Artificial Sequence Synthetic peptide
DHSSLKR

SEQ ID NO: 16 - protein, 7 aa, Artificial Sequence
  Description of Artificial Sequence Synthetic peptide
HKSSLTR

SEQ ID NO: 17 - protein, 7 aa, Artificial Sequence
  Description of Artificial Sequence Synthetic peptide
RREHLTI

SEQ ID NO: 18 - protein, 7 aa, Artificial Sequence
  Description of Artificial Sequence Synthetic peptide
QREHLTT

SEQ ID NO: 19 - protein, 7 aa, Artificial Sequence
  Description of Artificial Sequence Synthetic peptide
RKHHLGR

SEQ ID NO: 20 - protein, 7 aa, Artificial Sequence
  Description of Artificial Sequence Synthetic peptide
KKDHLHR

SEQ ID NO: 21 - protein, 7 aa, Artificial Sequence
  Description of Artificial Sequence Synthetic peptide
RKHHLGR

SEQ ID NO: 22 - protein, 7 aa, Artificial Sequence
  Description of Artificial Sequence Synthetic peptide
KGDHLRR

SEQ ID NO: 23 - protein, 7 aa, Artificial Sequence
  Description of Artificial Sequence Synthetic peptide
RNTHLAR

SEQ ID NO: 24 - protein, 7 aa, Artificial Sequence
  Description of Artificial Sequence Synthetic peptide
KRERLDR

SEQ ID NO: 25 - protein, 7 aa, Artificial Sequence
  Description of Artificial Sequence Synthetic peptide
KKDHLHR

SEQ ID NO: 26 - protein, 7 aa, Artificial Sequence
  Description of Artificial Sequence Synthetic peptide
KKDHLHR

SEQ ID NO: 27 - protein, 7 aa, Artificial Sequence
  Description of Artificial Sequence Synthetic peptide
RGNHLRR

SEQ ID NO: 28 - protein, 7 aa, Artificial Sequence
  Description of Artificial Sequence Synthetic peptide
RRAHLQN

SEQ ID NO: 29 - protein, 7 aa, Artificial Sequence
  Description of Artificial Sequence Synthetic peptide
KGDHLRR

SEQ ID NO: 30 - protein, 7 aa, Artificial Sequence
  Description of Artificial Sequence Synthetic peptide
KKDHLHR

SEQ ID NO: 31 - protein, 7 aa, Artificial Sequence
  Description of Artificial Sequence Synthetic peptide
KKDHLHR

SEQ ID NO: 32 - protein, 7 aa, Artificial Sequence
  Description of Artificial Sequence Synthetic peptide
KRERLER

SEQ ID NO: 33 - protein, 7 aa, Artificial Sequence
  Description of Artificial Sequence Synthetic peptide
KKDHLHR

SEQ ID NO: 34 - protein, 7 aa, Artificial Sequence
  Description of Artificial Sequence Synthetic peptide
KSNHLHV

SEQ ID NO: 35 - protein, 7 aa, Artificial Sequence
  Description of Artificial Sequence Synthetic peptide
TSAHLAR

SEQ ID NO: 36 - protein, 7 aa, Artificial Sequence
  Description of Artificial Sequence Synthetic peptide
RTDRLIR

SEQ ID NO: 37 - protein, 7 aa, Artificial Sequence
  Description of Artificial Sequence Synthetic peptide
RPSKLVL

SEQ ID NO: 38 - protein, 7 aa, Artificial Sequence
  Description of Artificial Sequence Synthetic peptide
THAHLTR

SEQ ID NO: 39 - protein, 7 aa, Artificial Sequence
  Description of Artificial Sequence Synthetic peptide
THAHLTR

SEQ ID NO: 40 - protein, 7 aa, Artificial Sequence
  Description of Artificial Sequence Synthetic peptide
RMERLDR

SEQ ID NO: 41 - protein, 7 aa, Artificial Sequence
  Description of Artificial Sequence Synthetic peptide
RPAKLVL

SEQ ID NO: 42 - protein, 7 aa, Artificial Sequence
  Description of Artificial Sequence Synthetic peptide
DKTKLRV

SEQ ID NO: 43 - protein, 7 aa, Artificial Sequence
  Description of Artificial Sequence Synthetic peptide
DKTKLNV

SEQ ID NO: 44 - protein, 7 aa, Artificial Sequence
  Description of Artificial Sequence Synthetic peptide
RTDRLIR

SEQ ID NO: 45 - protein, 7 aa, Artificial Sequence
  Description of Artificial Sequence Synthetic peptide
DNAHLAR

SEQ ID NO: 46 - protein, 7 aa, Artificial Sequence
  Description of Artificial Sequence Synthetic peptide
RPAKLVL

SEQ ID NO: 47 - protein, 7 aa, Artificial Sequence
  Description of Artificial Sequence Synthetic peptide
THAHLTR

SEQ ID NO: 48 - protein, 7 aa, Artificial Sequence
  Description of Artificial Sequence Synthetic peptide
THAHLTR

SEQ ID NO: 49 - protein, 7 aa, Artificial Sequence
  Description of Artificial Sequence Synthetic peptide
RPSKLVL

SEQ ID NO: 50 - protein, 7 aa, Artificial Sequence
  Description of Artificial Sequence Synthetic peptide
RSTHLRV

SEQ ID NO: 51 - protein, 7 aa, Artificial Sequence
  Description of Artificial Sequence Synthetic peptide
TNSKLTR

SEQ ID NO: 52 - protein, 7 aa, Artificial Sequence
  Description of Artificial Sequence Synthetic peptide
VPSKLLR

SEQ ID NO: 53 - protein, 7 aa, Artificial Sequence
  Description of Artificial Sequence Synthetic peptide
SKHKLER

SEQ ID NO: 54 - protein, 7 aa, Artificial Sequence
  Description of Artificial Sequence Synthetic peptide
VPSKLKR

SEQ ID NO: 55 - protein, 7 aa, Artificial Sequence
  Description of Artificial Sequence Synthetic peptide
SPSKLVR

SEQ ID NO: 56 - protein, 7 aa, Artificial Sequence
  Description of Artificial Sequence Synthetic peptide
TRAKLHI

SEQ ID NO: 57 - protein, 7 aa, Artificial Sequence
  Description of Artificial Sequence Synthetic peptide
APSKLDR

SEQ ID NO: 58 - protein, 7 aa, Artificial Sequence
  Description of Artificial Sequence Synthetic peptide
SPSKLAR

SEQ ID NO: 59 - protein, 7 aa, Artificial Sequence
  Description of Artificial Sequence Synthetic peptide
VPSKLAR

SEQ ID NO: 60 - protein, 7 aa, Artificial Sequence
  Description of Artificial Sequence Synthetic peptide
VPSKLLR

SEQ ID NO: 61 - protein, 7 aa, Artificial Sequence
  Description of Artificial Sequence Synthetic peptide
TPSKLDR

SEQ ID NO: 62 - protein, 7 aa, Artificial Sequence
  Description of Artificial Sequence Synthetic peptide
APSKLAR

SEQ ID NO: 63 - protein, 7 aa, Artificial Sequence
  Description of Artificial Sequence Synthetic peptide
SPSKLVR

SEQ ID NO: 64 - protein, 7 aa, Artificial Sequence
  Description of Artificial Sequence Synthetic peptide
SPSKLVR

SEQ ID NO: 65 - protein, 7 aa, Artificial Sequence
  Description of Artificial Sequence Synthetic peptide
AKSKLDR

SEQ ID NO: 66 - protein, 7 aa, Artificial Sequence
  Description of Artificial Sequence Synthetic peptide
APSKLKR

SEQ ID NO: 67 - protein, 7 aa, Artificial Sequence
  Description of Artificial Sequence Synthetic peptide
APSKLKR

SEQ ID NO: 68 - protein, 7 aa, Artificial Sequence
  Description of Artificial Sequence Synthetic peptide
RQSRLQR

SEQ ID NO: 69 - protein, 7 aa, Artificial Sequence
  Description of Artificial Sequence Synthetic peptide
IPNHLAR

SEQ ID NO: 70 - protein, 7 aa, Artificial Sequence
  Description of Artificial Sequence Synthetic peptide
RQSRLQR

SEQ ID NO: 71 - protein, 7 aa, Artificial Sequence
  Description of Artificial Sequence Synthetic peptide
MKHHLAR

SEQ ID NO: 72 - protein, 7 aa, Artificial Sequence
  Description of Artificial Sequence Synthetic peptide
TRQKLET

SEQ ID NO: 73 - protein, 7 aa, Artificial Sequence
  Description of Artificial Sequence Synthetic peptide
TKQRLEV

SEQ ID NO: 74 - protein, 7 aa, Artificial Sequence
  Description of Artificial Sequence Synthetic peptide
MKHHLDA

SEQ ID NO: 75 - protein, 7 aa, Artificial Sequence
  Description of Artificial Sequence Synthetic peptide
TKQKLQT

SEQ ID NO: 76 - protein, 7 aa, Artificial Sequence
  Description of Artificial Sequence Synthetic peptide
TTTKLAI

SEQ ID NO: 77 - protein, 7 aa, Artificial Sequence
  Description of Artificial Sequence Synthetic peptide
RQSRLQR

SEQ ID NO: 78 - protein, 7 aa, Artificial Sequence
  Description of Artificial Sequence Synthetic peptide
RRQKLTI

SEQ ID NO: 79 - protein, 7 aa, Artificial Sequence
  Description of Artificial Sequence Synthetic peptide
IPNHLAR

SEQ ID NO: 80 - protein, 7 aa, Artificial Sequence
  Description of Artificial Sequence Synthetic peptide
TRTRLVI

SEQ ID NO: 81 - protein, 7 aa, Artificial Sequence
  Description of Artificial Sequence Synthetic peptide
RRSRLVR

SEQ ID NO: 82 - protein, 7 aa, Artificial Sequence
  Description of Artificial Sequence Synthetic peptide
RRQKLTI

SEQ ID NO: 83 - protein, 7 aa, Artificial Sequence
  Description of Artificial Sequence Synthetic peptide
RNTNLTR

SEQ ID NO: 84 - protein, 7 aa, Artificial Sequence
  Description of Artificial Sequence Synthetic peptide
KHSNLTR

SEQ ID NO: 85 - protein, 7 aa, Artificial Sequence
  Description of Artificial Sequence Synthetic peptide
RNTNLTR

SEQ ID NO: 86 - protein, 7 aa, Artificial Sequence
  Description of Artificial Sequence Synthetic peptide
RQMNLDR

SEQ ID NO: 87 - protein, 7 aa, Artificial Sequence
  Description of Artificial Sequence Synthetic peptide
KHSNLTR

SEQ ID NO: 88 - protein, 7 aa, Artificial Sequence
  Description of Artificial Sequence Synthetic peptide
RTHNLTR

SEQ ID NO: 89 - protein, 7 aa, Artificial Sequence
  Description of Artificial Sequence Synthetic peptide
TNNNLAR

SEQ ID NO: 90 - protein, 7 aa, Artificial Sequence
  Description of Artificial Sequence Synthetic peptide
RMSNLDR

SEQ ID NO: 91 - protein, 7 aa, Artificial Sequence
  Description of Artificial Sequence Synthetic peptide
KHSNLAR

SEQ ID NO: 92 - protein, 7 aa, Artificial Sequence
  Description of Artificial Sequence Synthetic peptide
KKTNLTR

SEQ ID NO: 93 - protein, 7 aa, Artificial Sequence
  Description of Artificial Sequence Synthetic peptide
KHSNLTR

SEQ ID NO: 94 - protein, 7 aa, Artificial Sequence
  Description of Artificial Sequence Synthetic peptide
KHSNLTR

SEQ ID NO: 95 - protein, 7 aa, Artificial Sequence
  Description of Artificial Sequence Synthetic peptide
KHSNLTR

SEQ ID NO: 96 - protein, 7 aa, Artificial Sequence
  Description of Artificial Sequence Synthetic peptide
RTHNLKR

SEQ ID NO: 97 - protein, 7 aa, Artificial Sequence
  Description of Artificial Sequence Synthetic peptide
RPHNLLR

SEQ ID NO: 98 - protein, 7 aa, Artificial Sequence
  Description of Artificial Sequence Synthetic peptide
QLSNLTR

SEQ ID NO: 99 - protein, 7 aa, Artificial Sequence
  Description of Artificial Sequence Synthetic peptide
QRSNLAR

SEQ ID NO: 100 - protein, 7 aa, Artificial Sequence
  Description of Artificial Sequence Synthetic peptide
HRPNLTR

SEQ ID NO: 101 - protein, 7 aa, Artificial Sequence
  Description of Artificial Sequence Synthetic peptide
RKPNLLR

SEQ ID NO: 102 - protein, 7 aa, Artificial Sequence
  Description of Artificial Sequence Synthetic peptide
QASNLLR

SEQ ID NO: 103 - protein, 7 aa, Artificial Sequence
  Description of Artificial Sequence Synthetic peptide
QASNLTR

SEQ ID NO: 104 - protein, 7 aa, Artificial Sequence
  Description of Artificial Sequence Synthetic peptide
QRSNLAR

SEQ ID NO: 105 - protein, 7 aa, Artificial Sequence
  Description of Artificial Sequence Synthetic peptide
RKPNLLR

SEQ ID NO: 106 - protein, 7 aa, Artificial Sequence
  Description of Artificial Sequence Synthetic peptide
QQSNLSR

SEQ ID NO: 107 - protein, 7 aa, Artificial Sequence
  Description of Artificial Sequence Synthetic peptide
QGSNLAR

SEQ ID NO: 108 - protein, 7 aa, Artificial Sequence
  Description of Artificial Sequence Synthetic peptide
QASNLAR

SEQ ID NO: 109 - protein, 7 aa, Artificial Sequence
  Description of Artificial Sequence Synthetic peptide
TTTNLRR

SEQ ID NO: 110 - protein, 7 aa, Artificial Sequence
  Description of Artificial Sequence Synthetic peptide
HKPNLHR

SEQ ID NO: 111 - protein, 7 aa, Artificial Sequence
  Description of Artificial Sequence Synthetic peptide
HRPNLTR

SEQ ID NO: 112 - protein, 7 aa, Artificial Sequence
  Description of Artificial Sequence Synthetic peptide
QRTNLQR

SEQ ID NO: 113 - protein, 7 aa, Artificial Sequence
  Description of Artificial Sequence Synthetic peptide
QRSNLAR

SEQ ID NO: 114 - protein, 7 aa, Artificial Sequence
  Description of Artificial Sequence Synthetic peptide
QNANLKR

SEQ ID NO: 115 - protein, 7 aa, Artificial Sequence
  Description of Artificial Sequence Synthetic peptide
EEANLRR

SEQ ID NO: 116 - protein, 7 aa, Artificial Sequence
  Description of Artificial Sequence Synthetic peptide
DEANLRR

SEQ ID NO: 117 - protein, 7 aa, Artificial Sequence
  Description of Artificial Sequence Synthetic peptide
DPSNLRR

SEQ ID NO: 118 - protein, 7 aa, Artificial Sequence
  Description of Artificial Sequence Synthetic peptide
EEANLRR

SEQ ID NO: 119 - protein, 7 aa, Artificial Sequence
  Description of Artificial Sequence Synthetic peptide
DEANLRR

SEQ ID NO: 120 - protein, 7 aa, Artificial Sequence
  Description of Artificial Sequence Synthetic peptide
DEANLRR

SEQ ID NO: 121 - protein, 7 aa, Artificial Sequence
  Description of Artificial Sequence Synthetic peptide
EESNLRR

SEQ ID NO: 122 - protein, 7 aa, Artificial Sequence
  Description of Artificial Sequence Synthetic peptide
EEVNLRR

SEQ ID NO: 123 - protein, 7 aa, Artificial Sequence
  Description of Artificial Sequence Synthetic peptide
DPSNLIR

SEQ ID NO: 124 - protein, 7 aa, Artificial Sequence
  Description of Artificial Sequence Synthetic peptide
EEVNLRR

SEQ ID NO: 125 - protein, 7 aa, Artificial Sequence
  Description of Artificial Sequence Synthetic peptide
DPSNLRR

SEQ ID NO: 126 - protein, 7 aa, Artificial Sequence
  Description of Artificial Sequence Synthetic peptide
DLSNLKR

SEQ ID NO: 127 - protein, 7 aa, Artificial Sequence
  Description of Artificial Sequence Synthetic peptide
DEANLRR

SEQ ID NO: 128 - protein, 7 aa, Artificial Sequence
  Description of Artificial Sequence Synthetic peptide
DPANLRR

SEQ ID NO: 129 - protein, 7 aa, Artificial Sequence
  Description of Artificial Sequence Synthetic peptide
DDANLRR

SEQ ID NO: 130 - protein, 7 aa, Artificial Sequence
  Description of Artificial Sequence Synthetic peptide
EQANLRR

SEQ ID NO: 131 - protein, 7 aa, Artificial Sequence
  Description of Artificial Sequence Synthetic peptide
TRQRLRI

SEQ ID NO: 132 - protein, 7 aa, Artificial Sequence
  Description of Artificial Sequence Synthetic peptide
TSQMLVV

SEQ ID NO: 133 - protein, 7 aa, Artificial Sequence
  Description of Artificial Sequence Synthetic peptide
TKQRLVV

SEQ ID NO: 134 - protein, 7 aa, Artificial Sequence
  Description of Artificial Sequence Synthetic peptide
SKQALAV

SEQ ID NO: 135 - protein, 7 aa, Artificial Sequence
  Description of Artificial Sequence Synthetic peptide
TRQRLAI

SEQ ID NO: 136 - protein, 7 aa, Artificial Sequence
  Description of Artificial Sequence Synthetic peptide
TGQQLRV

SEQ ID NO: 137 - protein, 7 aa, Artificial Sequence
  Description of Artificial Sequence Synthetic peptide
KRHTLTR

SEQ ID NO: 138 - protein, 7 aa, Artificial Sequence
  Description of Artificial Sequence Synthetic peptide
KNNDLTR

SEQ ID NO: 139 - protein, 7 aa, Artificial Sequence
  Description of Artificial Sequence Synthetic peptide
RRHGLDR

SEQ ID NO: 140 - protein, 7 aa, Artificial Sequence
  Description of Artificial Sequence Synthetic peptide
RRLTLLR

SEQ ID NO: 141 - protein, 7 aa, Artificial Sequence
  Description of Artificial Sequence Synthetic peptide
RKLGLLR

SEQ ID NO: 142 - protein, 7 aa, Artificial Sequence
  Description of Artificial Sequence Synthetic peptide
RLRDLPR

SEQ ID NO: 143 - protein, 7 aa, Artificial Sequence
  Description of Artificial Sequence Synthetic peptide
KHDTLHR

SEQ ID NO: 144 - protein, 7 aa, Artificial Sequence
  Description of Artificial Sequence Synthetic peptide
RNLTLAR

SEQ ID NO: 145 - protein, 7 aa, Artificial Sequence
  Description of Artificial Sequence Synthetic peptide
RSNTLLR

SEQ ID NO: 146 - protein, 7 aa, Artificial Sequence
  Description of Artificial Sequence Synthetic peptide
RRHTLTR

SEQ ID NO: 147 - protein, 7 aa, Artificial Sequence
  Description of Artificial Sequence Synthetic peptide
RLRDLPR

SEQ ID NO: 148 - protein, 7 aa, Artificial Sequence
  Description of Artificial Sequence Synthetic peptide
RKGTLDR

SEQ ID NO: 149 - protein, 7 aa, Artificial Sequence
  Description of Artificial Sequence Synthetic peptide
RAHTLRR

SEQ ID NO: 150 - protein, 7 aa, Artificial Sequence
  Description of Artificial Sequence Synthetic peptide
KADTLVR

SEQ ID NO: 151 - protein, 7 aa, Artificial Sequence
  Description of Artificial Sequence Synthetic peptide
RRHGLDR

SEQ ID NO: 152 - protein, 7 aa, Artificial Sequence
  Description of Artificial Sequence Synthetic peptide
RNLTLVR

SEQ ID NO: 153 - protein, 7 aa, Artificial Sequence
  Description of Artificial Sequence Synthetic peptide
DRSQLAR

SEQ ID NO: 154 - protein, 7 aa, Artificial Sequence
  Description of Artificial Sequence Synthetic peptide
KNTRLSV

SEQ ID NO: 155 - protein, 7 aa, Artificial Sequence
  Description of Artificial Sequence Synthetic peptide
RRQELHR

SEQ ID NO: 156 - protein, 7 aa, Artificial Sequence
  Description of Artificial Sequence Synthetic peptide
DKAQLGR

SEQ ID NO: 157 - protein, 7 aa, Artificial Sequence
  Description of Artificial Sequence Synthetic peptide
RRVDLLR

SEQ ID NO: 158 - protein, 7 aa, Artificial Sequence
  Description of Artificial Sequence Synthetic peptide
RGQELRR

SEQ ID NO: 159 - protein, 7 aa, Artificial Sequence
  Description of Artificial Sequence Synthetic peptide
LKHSLLR

SEQ ID NO: 160 - protein, 7 aa, Artificial Sequence
  Description of Artificial Sequence Synthetic peptide
LRHSLSR

SEQ ID NO: 161 - protein, 7 aa, Artificial Sequence
  Description of Artificial Sequence Synthetic peptide
SPEQLAR

SEQ ID NO: 162 - protein, 7 aa, Artificial Sequence
  Description of Artificial Sequence Synthetic peptide
QRGTLNR

SEQ ID NO: 163 - protein, 7 aa, Artificial Sequence
  Description of Artificial Sequence Synthetic peptide
LNHTLKR

SEQ ID NO: 164 - protein, 7 aa, Artificial Sequence
  Description of Artificial Sequence Synthetic peptide
HNGTLKR

SEQ ID NO: 165 - protein, 7 aa, Artificial Sequence
  Description of Artificial Sequence Synthetic peptide
HNGTLKR

SEQ ID NO: 166 - protein, 7 aa, Artificial Sequence
  Description of Artificial Sequence Synthetic peptide
RRQELTR

SEQ ID NO: 167 - protein, 7 aa, Artificial Sequence
  Description of Artificial Sequence Synthetic peptide
RRQELKR

SEQ ID NO: 168 - protein, 7 aa, Artificial Sequence
  Description of Artificial Sequence Synthetic peptide
SHTVLTR

SEQ ID NO: 169 - protein, 7 aa, Artificial Sequence
  Description of Artificial Sequence Synthetic peptide
DSPTLRR

SEQ ID NO: 170 - protein, 7 aa, Artificial Sequence
  Description of Artificial Sequence Synthetic peptide
SNKDLTR

SEQ ID NO: 171 - protein, 7 aa, Artificial Sequence
  Description of Artificial Sequence Synthetic peptide
SKKSLTR

SEQ ID NO: 172 - protein, 7 aa, Artificial Sequence
  Description of Artificial Sequence Synthetic peptide
VRKDLTR

SEQ ID NO: 173 - protein, 7 aa, Artificial Sequence
  Description of Artificial Sequence Synthetic peptide
DSPTLRR

SEQ ID NO: 174 - protein, 7 aa, Artificial Sequence
  Description of Artificial Sequence Synthetic peptide
ERRGLDR

SEQ ID NO: 175 - protein, 7 aa, Artificial Sequence
  Description of Artificial Sequence Synthetic peptide
DGSTLNR

SEQ ID NO: 176 - protein, 7 aa, Artificial Sequence
  Description of Artificial Sequence Synthetic peptide
DPSTLRR

SEQ ID NO: 177 - protein, 7 aa, Artificial Sequence
  Description of Artificial Sequence Synthetic peptide
DPSTLRR

SEQ ID NO: 178 - protein, 7 aa, Artificial Sequence
  Description of Artificial Sequence Synthetic peptide
DGSTLRR

SEQ ID NO: 179 - protein, 7 aa, Artificial Sequence
  Description of Artificial Sequence Synthetic peptide
DRRTLDR

SEQ ID NO: 180 - protein, 7 aa, Artificial Sequence
  Description of Artificial Sequence Synthetic peptide
KRRDLDR

SEQ ID NO: 181 - protein, 7 aa, Artificial Sequence
  Description of Artificial Sequence Synthetic peptide
DGSTLRR

SEQ ID NO: 182 - protein, 7 aa, Artificial Sequence
  Description of Artificial Sequence Synthetic peptide
DSPTLRR

SEQ ID NO: 183 - protein, 7 aa, Artificial Sequence
  Description of Artificial Sequence Synthetic peptide
LKKDLLR

SEQ ID NO: 184 - protein, 7 aa, Artificial Sequence
  Description of Artificial Sequence Synthetic peptide
THSMLAR

SEQ ID NO: 185 - protein, 7 aa, Artificial Sequence
  Description of Artificial Sequence Synthetic peptide
TTQALRR

SEQ ID NO: 186 - protein, 7 aa, Artificial Sequence
  Description of Artificial Sequence Synthetic peptide
TSSGLTR

SEQ ID NO: 187 - protein, 7 aa, Artificial Sequence
  Description of Artificial Sequence Synthetic peptide
LRQTLAR

SEQ ID NO: 188 - protein, 7 aa, Artificial Sequence
  Description of Artificial Sequence Synthetic peptide
NKQALDR

SEQ ID NO: 189 - protein, 7 aa, Artificial Sequence
  Description of Artificial Sequence Synthetic peptide
QRQALDR

SEQ ID NO: 190 - protein, 7 aa, Artificial Sequence
  Description of Artificial Sequence Synthetic peptide
VRQGLTR

SEQ ID NO: 191 - protein, 7 aa, Artificial Sequence
  Description of Artificial Sequence Synthetic peptide
TKQVLDR

SEQ ID NO: 192 - protein, 7 aa, Artificial Sequence
  Description of Artificial Sequence Synthetic peptide
KHQTLQR

SEQ ID NO: 193 - protein, 7 aa, Artificial Sequence
  Description of Artificial Sequence Synthetic peptide
TKQILGR

SEQ ID NO: 194 - protein, 7 aa, Artificial Sequence
  Description of Artificial Sequence Synthetic peptide
LRTSLVR

SEQ ID NO: 195 - protein, 7 aa, Artificial Sequence
  Description of Artificial Sequence Synthetic peptide
MKNTLTR

SEQ ID NO: 196 - protein, 7 aa, Artificial Sequence
  Description of Artificial Sequence Synthetic peptide
TKPILVR

SEQ ID NO: 197 - protein, 7 aa, Artificial Sequence
  Description of Artificial Sequence Synthetic peptide
NGQGLRR

SEQ ID NO: 198 - protein, 7 aa, Artificial Sequence
  Description of Artificial Sequence Synthetic peptide
GATALKR

SEQ ID NO: 199 - protein, 7 aa, Artificial Sequence
  Description of Artificial Sequence Synthetic peptide
TKQILGR

SEQ ID NO: 200 - protein, 7 aa, Artificial Sequence
  Description of Artificial Sequence Synthetic peptide
TKPILVR

SEQ ID NO: 201 - protein, 7 aa, Artificial Sequence
  Description of Artificial Sequence Synthetic peptide
SRFTLGR

SEQ ID NO: 202 - protein, 7 aa, Artificial Sequence
  Description of Artificial Sequence Synthetic peptide
RNFILQR

SEQ ID NO: 203 - protein, 7 aa, Artificial Sequence
  Description of Artificial Sequence Synthetic peptide
SRFTLGR

SEQ ID NO: 204 - protein, 7 aa, Artificial Sequence
  Description of Artificial Sequence Synthetic peptide
RRFILSR

SEQ ID NO: 205 - protein, 7 aa, Artificial Sequence
  Description of Artificial Sequence Synthetic peptide
RRHILDR

SEQ ID NO: 206 - protein, 7 aa, Artificial Sequence
  Description of Artificial Sequence Synthetic peptide
RTSSLKR

SEQ ID NO: 207 - protein, 7 aa, Artificial Sequence
  Description of Artificial Sequence Synthetic peptide
RASVLDI

SEQ ID NO: 208 - protein, 7 aa, Artificial Sequence
  Description of Artificial Sequence Synthetic peptide
RKHILIH

SEQ ID NO: 209 - protein, 7 aa, Artificial Sequence
  Description of Artificial Sequence Synthetic peptide
RNFILAR

SEQ ID NO: 210 - protein, 7 aa, Artificial Sequence
  Description of Artificial Sequence Synthetic peptide
RSHILTN

SEQ ID NO: 211 - protein, 7 aa, Artificial Sequence
  Description of Artificial Sequence Synthetic peptide
RTSSLKR

SEQ ID NO: 212 - protein, 7 aa, Artificial Sequence
  Description of Artificial Sequence Synthetic peptide
RTSSLKR

SEQ ID NO: 213 - protein, 7 aa, Artificial Sequence
  Description of Artificial Sequence Synthetic peptide
RNFVLAR

SEQ ID NO: 214 - protein, 7 aa, Artificial Sequence
  Description of Artificial Sequence Synthetic peptide
RKHILIH

SEQ ID NO: 215 - protein, 7 aa, Artificial Sequence
  Description of Artificial Sequence Synthetic peptide
RNFILQR

SEQ ID NO: 216 - protein, 7 aa, Artificial Sequence
  Description of Artificial Sequence Synthetic peptide
RNFILQR

SEQ ID NO: 217 - protein, 7 aa, Artificial Sequence
  Description of Artificial Sequence Synthetic peptide
RNFILQR

SEQ ID NO: 218 - protein, 7 aa, Artificial Sequence
  Description of Artificial Sequence Synthetic peptide
QQSSLLR

SEQ ID NO: 219 - protein, 7 aa, Artificial Sequence
  Description of Artificial Sequence Synthetic peptide
QQQALTR

SEQ ID NO: 220 - protein, 7 aa, Artificial Sequence
  Description of Artificial Sequence Synthetic peptide
QQSSLLR

SEQ ID NO: 221 - protein, 7 aa, Artificial Sequence
  Description of Artificial Sequence Synthetic peptide
QQQALKR

SEQ ID NO: 222 - protein, 7 aa, Artificial Sequence
  Description of Artificial Sequence Synthetic peptide
QQQALVR

SEQ ID NO: 223 - protein, 7 aa, Artificial Sequence
  Description of Artificial Sequence Synthetic peptide
QKQALDR

SEQ ID NO: 224 - protein, 7 aa, Artificial Sequence
  Description of Artificial Sequence Synthetic peptide
QQQALVR

SEQ ID NO: 225 - protein, 7 aa, Artificial Sequence
  Description of Artificial Sequence Synthetic peptide
QQQALVR

SEQ ID NO: 226 - protein, 7 aa, Artificial Sequence
  Description of Artificial Sequence Synthetic peptide
QQQALKR

SEQ ID NO: 227 - protein, 7 aa, Artificial Sequence
  Description of Artificial Sequence Synthetic peptide
QKQALTR

SEQ ID NO: 228 - protein, 7 aa, Artificial Sequence
  Description of Artificial Sequence Synthetic peptide
QKQALTR

SEQ ID NO: 229 - protein, 7 aa, Artificial Sequence
  Description of Artificial Sequence Synthetic peptide
QQSSLLR

SEQ ID NO: 230 - protein, 7 aa, Artificial Sequence
  Description of Artificial Sequence Synthetic peptide
QQQALKR

SEQ ID NO: 231 - protein, 7 aa, Artificial Sequence
  Description of Artificial Sequence Synthetic peptide
QQQALKR

SEQ ID NO: 232 - protein, 7 aa, Artificial Sequence
  Description of Artificial Sequence Synthetic peptide
QQQALKR

SEQ ID NO: 233 - protein, 7 aa, Artificial Sequence
  Description of Artificial Sequence Synthetic peptide
TKSLLAR

SEQ ID NO: 234 - protein, 7 aa, Artificial Sequence
  Description of Artificial Sequence Synthetic peptide
TSTLLKR

SEQ ID NO: 235 - protein, 7 aa, Artificial Sequence
  Description of Artificial Sequence Synthetic peptide
TGAVLTR

SEQ ID NO: 236 - protein, 7 aa, Artificial Sequence
  Description of Artificial Sequence Synthetic peptide
TRAVLRR

SEQ ID NO: 237 - protein, 7 aa, Artificial Sequence
  Description of Artificial Sequence Synthetic peptide
NTSLLRR

SEQ ID NO: 238 - protein, 7 aa, Artificial Sequence
  Description of Artificial Sequence Synthetic peptide
TSTLLNR

SEQ ID NO: 239 - protein, 7 aa, Artificial Sequence
  Description of Artificial Sequence Synthetic peptide
TKSLLAR

SEQ ID NO: 240 - protein, 7 aa, Artificial Sequence
  Description of Artificial Sequence Synthetic peptide
TKKILTV

SEQ ID NO: 241 - protein, 7 aa, Artificial Sequence
  Description of Artificial Sequence Synthetic peptide
TSTLLNR

SEQ ID NO: 242 - protein, 7 aa, Artificial Sequence
  Description of Artificial Sequence Synthetic peptide
TGAVLTR

SEQ ID NO: 243 - protein, 7 aa, Artificial Sequence
  Description of Artificial Sequence Synthetic peptide
APSSLRR

SEQ ID NO: 244 - protein, 7 aa, Artificial Sequence
  Description of Artificial Sequence Synthetic peptide
TRAVLRR

SEQ ID NO: 245 - protein, 7 aa, Artificial Sequence
  Description of Artificial Sequence Synthetic peptide
TMAVLRR

SEQ ID NO: 246 - protein, 7 aa, Artificial Sequence
  Description of Artificial Sequence Synthetic peptide
TSTILAR

SEQ ID NO: 247 - protein, 7 aa, Artificial Sequence
  Description of Artificial Sequence Synthetic peptide
TGAVLRR

SEQ ID NO: 248 - protein, 7 aa, Artificial Sequence
  Description of Artificial Sequence Synthetic peptide
TKKILTV

SEQ ID NO: 249 - protein, 7 aa, Artificial Sequence
  Description of Artificial Sequence Synthetic peptide
IKAILTR

SEQ ID NO: 250 - protein, 7 aa, Artificial Sequence
  Description of Artificial Sequence Synthetic peptide
TTALLKR

SEQ ID NO: 251 - protein, 7 aa, Artificial Sequence
  Description of Artificial Sequence Synthetic peptide
TSTLLKR

SEQ ID NO: 252 - protein, 7 aa, Artificial Sequence
  Description of Artificial Sequence Synthetic peptide
AATALRR

SEQ ID NO: 253 - protein, 7 aa, Artificial Sequence
  Description of Artificial Sequence Synthetic peptide
TTTVLAR

SEQ ID NO: 254 - protein, 7 aa, Artificial Sequence
  Description of Artificial Sequence Synthetic peptide
TSTLLKR

SEQ ID NO: 255 - protein, 7 aa, Artificial Sequence
  Description of Artificial Sequence Synthetic peptide
THTVLAR

SEQ ID NO: 256 - protein, 7 aa, Artificial Sequence
  Description of Artificial Sequence Synthetic peptide
TNQALGV

SEQ ID NO: 257 - protein, 7 aa, Artificial Sequence
  Description of Artificial Sequence Synthetic peptide
MTSSLRR

SEQ ID NO: 258 - protein, 7 aa, Artificial Sequence
  Description of Artificial Sequence Synthetic peptide
TTTVLAR

SEQ ID NO: 259 - protein, 7 aa, Artificial Sequence
  Description of Artificial Sequence Synthetic peptide
QATLLRR

SEQ ID NO: 260 - protein, 7 aa, Artificial Sequence
  Description of Artificial Sequence Synthetic peptide
MNSVLKR

SEQ ID NO: 261 - protein, 7 aa, Artificial Sequence
  Description of Artificial Sequence Synthetic peptide
TSTRLDI

SEQ ID NO: 262 - protein, 7 aa, Artificial Sequence
  Description of Artificial Sequence Synthetic peptide
TKPVLKI

SEQ ID NO: 263 - protein, 7 aa, Artificial Sequence
  Description of Artificial Sequence Synthetic peptide
TNSVLGR

SEQ ID NO: 264 - protein, 7 aa, Artificial Sequence
  Description of Artificial Sequence Synthetic peptide
RPHHLDA

SEQ ID NO: 265 - protein, 7 aa, Artificial Sequence
  Description of Artificial Sequence Synthetic peptide
RRAHLLS

SEQ ID NO: 266 - protein, 7 aa, Artificial Sequence
  Description of Artificial Sequence Synthetic peptide
RMAHLHA

SEQ ID NO: 267 - protein, 7 aa, Artificial Sequence
  Description of Artificial Sequence Synthetic peptide
RRAHLLN

SEQ ID NO: 268 - protein, 7 aa, Artificial Sequence
  Description of Artificial Sequence Synthetic peptide
RGNHLVV

SEQ ID NO: 269 - protein, 7 aa, Artificial Sequence
  Description of Artificial Sequence Synthetic peptide
RSSHLKM

SEQ ID NO: 270 - protein, 7 aa, Artificial Sequence
  Description of Artificial Sequence Synthetic peptide
RNEHLKV

SEQ ID NO: 271 - protein, 7 aa, Artificial Sequence
  Description of Artificial Sequence Synthetic peptide
RSSHLKM

SEQ ID NO: 272 - protein, 7 aa, Artificial Sequence
  Description of Artificial Sequence Synthetic peptide
RRAHLRQ

SEQ ID NO: 273 - protein, 7 aa, Artificial Sequence
  Description of Artificial Sequence Synthetic peptide
RRTHLRV

SEQ ID NO: 274 - protein, 7 aa, Artificial Sequence
  Description of Artificial Sequence Synthetic peptide
HRTNLIA

SEQ ID NO: 275 - protein, 7 aa, Artificial Sequence
  Description of Artificial Sequence Synthetic peptide
GGTALVM

SEQ ID NO: 276 - protein, 7 aa, Artificial Sequence
  Description of Artificial Sequence Synthetic peptide
GASALRS

SEQ ID NO: 277 - protein, 7 aa, Artificial Sequence
  Description of Artificial Sequence Synthetic peptide
GHTALRN

SEQ ID NO: 278 - protein, 7 aa, Artificial Sequence
  Description of Artificial Sequence Synthetic peptide
GASALRQ

SEQ ID NO: 279 - protein, 7 aa, Artificial Sequence
  Description of Artificial Sequence Synthetic peptide
GHTALRN

SEQ ID NO: 280 - protein, 7 aa, Artificial Sequence
  Description of Artificial Sequence Synthetic peptide
GHTALRN

SEQ ID NO: 281 - protein, 7 aa, Artificial Sequence
  Description of Artificial Sequence Synthetic peptide
GHTALAL

SEQ ID NO: 282 - protein, 7 aa, Artificial Sequence
  Description of Artificial Sequence Synthetic peptide
GHTALRN

SEQ ID NO: 283 - protein, 7 aa, Artificial Sequence
  Description of Artificial Sequence Synthetic peptide
GHTALRH

SEQ ID NO: 284 - protein, 7 aa, Artificial Sequence
  Description of Artificial Sequence Synthetic peptide
GGTALRM

SEQ ID NO: 285 - protein, 7 aa, Artificial Sequence
  Description of Artificial Sequence Synthetic peptide
GPTALVN

SEQ ID NO: 286 - protein, 7 aa, Artificial Sequence
  Description of Artificial Sequence Synthetic peptide
KNNDLTR

SEQ ID NO: 287 - protein, 7 aa, Artificial Sequence
  Description of Artificial Sequence Synthetic peptide
KRIDLQR

SEQ ID NO: 288 - protein, 7 aa, Artificial Sequence
  Description of Artificial Sequence Synthetic peptide
KNNDLTR

SEQ ID NO: 289 - protein, 7 aa, Artificial Sequence
  Description of Artificial Sequence Synthetic peptide
RKHDLNM

SEQ ID NO: 290 - protein, 7 aa, Artificial Sequence
  Description of Artificial Sequence Synthetic peptide
RRQTLRQ

SEQ ID NO: 291 - protein, 7 aa, Artificial Sequence
  Description of Artificial Sequence Synthetic peptide
KGNDLTR

SEQ ID NO: 292 - protein, 7 aa, Artificial Sequence
  Description of Artificial Sequence Synthetic peptide
KGNDLTR

SEQ ID NO: 293 - protein, 7 aa, Artificial Sequence
  Description of Artificial Sequence Synthetic peptide
RSQTLAQ

SEQ ID NO: 294 - protein, 7 aa, Artificial Sequence
  Description of Artificial Sequence Synthetic peptide
RSQTLAQ

SEQ ID NO: 295 - protein, 7 aa, Artificial Sequence
  Description of Artificial Sequence Synthetic peptide
RNITLVR

SEQ ID NO: 296 - protein, 7 aa, Artificial Sequence
  Description of Artificial Sequence Synthetic peptide
RNITLVR

SEQ ID NO: 297 - protein, 7 aa, Artificial Sequence
  Description of Artificial Sequence Synthetic peptide
RSHDLTV

SEQ ID NO: 298 - protein, 7 aa, Artificial Sequence
  Description of Artificial Sequence Synthetic peptide
RRRNLTL

SEQ ID NO: 299 - protein, 7 aa, Artificial Sequence
  Description of Artificial Sequence Synthetic peptide
RSRNLLL

SEQ ID NO: 300 - protein, 7 aa, Artificial Sequence
  Description of Artificial Sequence Synthetic peptide
RRRNLHL

SEQ ID NO: 301 - protein, 7 aa, Artificial Sequence
  Description of Artificial Sequence Synthetic peptide
RRRNLTL

SEQ ID NO: 302 - protein, 7 aa, Artificial Sequence
  Description of Artificial Sequence Synthetic peptide
RSRNLDI

SEQ ID NO: 303 - protein, 7 aa, Artificial Sequence
  Description of Artificial Sequence Synthetic peptide
RKRNLIM

SEQ ID NO: 304 - protein, 7 aa, Artificial Sequence
  Description of Artificial Sequence Synthetic peptide
RSRNLTL

SEQ ID NO: 305 - protein, 7 aa, Artificial Sequence
  Description of Artificial Sequence Synthetic peptide
RNRNLVL

SEQ ID NO: 306 - protein, 7 aa, Artificial Sequence
  Description of Artificial Sequence Synthetic peptide
RGRNLEM

SEQ ID NO: 307 - protein, 7 aa, Artificial Sequence
  Description of Artificial Sequence Synthetic peptide
RARNLTL

SEQ ID NO: 308 - protein, 7 aa, Artificial Sequence
  Description of Artificial Sequence Synthetic peptide
RKRNLIM

SEQ ID NO: 309 - protein, 7 aa, Artificial Sequence
  Description of Artificial Sequence Synthetic peptide
RMRNLII

SEQ ID NO: 310 - protein, 7 aa, Artificial Sequence
  Description of Artificial Sequence Synthetic peptide
RKQHLTL

SEQ ID NO: 311 - protein, 7 aa, Artificial Sequence
  Description of Artificial Sequence Synthetic peptide
RRQHLQY

SEQ ID NO: 312 - protein, 7 aa, Artificial Sequence
  Description of Artificial Sequence Synthetic peptide
RHQHLKL

SEQ ID NO: 313 - protein, 7 aa, Artificial Sequence
  Description of Artificial Sequence Synthetic peptide
RRQHLQY

SEQ ID NO: 314 - protein, 7 aa, Artificial Sequence
  Description of Artificial Sequence Synthetic peptide
RKQHLQL

SEQ ID NO: 315 - protein, 7 aa, Artificial Sequence
  Description of Artificial Sequence Synthetic peptide
KRQHLEY

SEQ ID NO: 316 - protein, 7 aa, Artificial Sequence
  Description of Artificial Sequence Synthetic peptide
RRQHLQY

SEQ ID NO: 317 - protein, 7 aa, Artificial Sequence
  Description of Artificial Sequence Synthetic peptide
RRQHLQY

SEQ ID NO: 318 - protein, 7 aa, Artificial Sequence
  Description of Artificial Sequence Synthetic peptide
RKQHLTL

SEQ ID NO: 319 - protein, 7 aa, Artificial Sequence
  Description of Artificial Sequence Synthetic peptide
KRQHLEY

SEQ ID NO: 320 - protein, 7 aa, Artificial Sequence
  Description of Artificial Sequence Synthetic peptide
RRQALEY

SEQ ID NO: 321 - protein, 7 aa, Artificial Sequence
  Description of Artificial Sequence Synthetic peptide
RKQHLQL

SEQ ID NO: 322 - protein, 7 aa, Artificial Sequence
  Description of Artificial Sequence Synthetic peptide
RKQHLVL

SEQ ID NO: 323 - protein, 7 aa, Artificial Sequence
  Description of Artificial Sequence Synthetic peptide
RRQHLQY

SEQ ID NO: 324 - protein, 7 aa, Artificial Sequence
  Description of Artificial Sequence Synthetic peptide
RGHNLLV

SEQ ID NO: 325 - protein, 7 aa, Artificial Sequence
  Description of Artificial Sequence Synthetic peptide
RRRNLQI

SEQ ID NO: 326 - protein, 7 aa, Artificial Sequence
  Description of Artificial Sequence Synthetic peptide
RSHNLRL

SEQ ID NO: 327 - protein, 7 aa, Artificial Sequence
  Description of Artificial Sequence Synthetic peptide
RSHNLRL

SEQ ID NO: 328 - protein, 7 aa, Artificial Sequence
  Description of Artificial Sequence Synthetic peptide
RSHNLKL

SEQ ID NO: 329 - protein, 7 aa, Artificial Sequence
  Description of Artificial Sequence Synthetic peptide
RSNNLRL

SEQ ID NO: 330 - protein, 7 aa, Artificial Sequence
  Description of Artificial Sequence Synthetic peptide
RAHNLLL

SEQ ID NO: 331 - protein, 7 aa, Artificial Sequence
  Description of Artificial Sequence Synthetic peptide
RGTNLRT

SEQ ID NO: 332 - protein, 7 aa, Artificial Sequence
  Description of Artificial Sequence Synthetic peptide
RSHNLRL

SEQ ID NO: 333 - protein, 7 aa, Artificial Sequence
  Description of Artificial Sequence Synthetic peptide
KNNDLLK

SEQ ID NO: 334 - protein, 7 aa, Artificial Sequence
  Description of Artificial Sequence Synthetic peptide
KNNDLLK

SEQ ID NO: 335 - protein, 7 aa, Artificial Sequence
  Description of Artificial Sequence Synthetic peptide
KNNDLLK

SEQ ID NO: 336 - protein, 7 aa, Artificial Sequence
  Description of Artificial Sequence Synthetic peptide
SKPNLKM

SEQ ID NO: 337 - protein, 7 aa, Artificial Sequence
  Description of Artificial Sequence Synthetic peptide
RANHLTI

SEQ ID NO: 338 - protein, 7 aa, Artificial Sequence
  Description of Artificial Sequence Synthetic peptide
RNDKLVP

SEQ ID NO: 339 - protein, 7 aa, Artificial Sequence
  Description of Artificial Sequence Synthetic peptide
RTEHLAR

SEQ ID NO: 340 - protein, 7 aa, Artificial Sequence
  Description of Artificial Sequence Synthetic peptide
RGDKLAL

SEQ ID NO: 341 - protein, 7 aa, Artificial Sequence
  Description of Artificial Sequence Synthetic peptide
RTEHLAR

SEQ ID NO: 342 - protein, 7 aa, Artificial Sequence
  Description of Artificial Sequence Synthetic peptide
RIDKLGG

SEQ ID NO: 343 - protein, 7 aa, Artificial Sequence
  Description of Artificial Sequence Synthetic peptide
RIDKLGG

SEQ ID NO: 344 - protein, 7 aa, Artificial Sequence
  Description of Artificial Sequence Synthetic peptide
RNHGLVR

SEQ ID NO: 345 - protein, 7 aa, Artificial Sequence
  Description of Artificial Sequence Synthetic peptide
RGDKLGP

SEQ ID NO: 346 - protein, 7 aa, Artificial Sequence
  Description of Artificial Sequence Synthetic peptide
RKHRLDG

SEQ ID NO: 347 - protein, 7 aa, Artificial Sequence
  Description of Artificial Sequence Synthetic peptide
RTEHLAR

SEQ ID NO: 348 - protein, 7 aa, Artificial Sequence
  Description of Artificial Sequence Synthetic peptide
RTEHLAR

SEQ ID NO: 349 - protein, 7 aa, Artificial Sequence
  Description of Artificial Sequence Synthetic peptide
RTEHLAR

SEQ ID NO: 350 - protein, 7 aa, Artificial Sequence
  Description of Artificial Sequence Synthetic peptide
RQGHLKR

SEQ ID NO: 351 - protein, 7 aa, Artificial Sequence
  Description of Artificial Sequence Synthetic peptide
RTEHLAR

SEQ ID NO: 352 - protein, 7 aa, Artificial Sequence
  Description of Artificial Sequence Synthetic peptide
RIDKLGG

SEQ ID NO: 353 - protein, 7 aa, Artificial Sequence
  Description of Artificial Sequence Synthetic peptide
RTEHLAR

SEQ ID NO: 354 - protein, 7 aa, Artificial Sequence
  Description of Artificial Sequence Synthetic peptide
QTTHLRR

SEQ ID NO: 355 - protein, 7 aa, Artificial Sequence
  Description of Artificial Sequence Synthetic peptide
QMSHLKR

SEQ ID NO: 356 - protein, 7 aa, Artificial Sequence
  Description of Artificial Sequence Synthetic peptide
QSQHLVR

SEQ ID NO: 357 - protein, 7 aa, Artificial Sequence
  Description of Artificial Sequence Synthetic peptide
QNSHLRR

SEQ ID NO: 358 - protein, 7 aa, Artificial Sequence
  Description of Artificial Sequence Synthetic peptide
QANHLSR

SEQ ID NO: 359 - protein, 7 aa, Artificial Sequence
  Description of Artificial Sequence Synthetic peptide
QTTHLSR

SEQ ID NO: 360 - protein, 7 aa, Artificial Sequence
  Description of Artificial Sequence Synthetic peptide
QGGHLKR

SEQ ID NO: 361 - protein, 7 aa, Artificial Sequence
  Description of Artificial Sequence Synthetic peptide
QTTHLSR

SEQ ID NO: 362 - protein, 7 aa, Artificial Sequence
  Description of Artificial Sequence Synthetic peptide
QTTHLSR

SEQ ID NO: 363 - protein, 7 aa, Artificial Sequence
  Description of Artificial Sequence Synthetic peptide
QKPHLSR

SEQ ID NO: 364 - protein, 7 aa, Artificial Sequence
  Description of Artificial Sequence Synthetic peptide
QNSHLRR

SEQ ID NO: 365 - protein, 7 aa, Artificial Sequence
  Description of Artificial Sequence Synthetic peptide
QKPHLSR

SEQ ID NO: 366 - protein, 7 aa, Artificial Sequence
  Description of Artificial Sequence Synthetic peptide
QTTHLSR

SEQ ID NO: 367 - protein, 7 aa, Artificial Sequence
  Description of Artificial Sequence Synthetic peptide
QTTHLSR

SEQ ID NO: 368 - protein, 7 aa, Artificial Sequence
  Description of Artificial Sequence Synthetic peptide
QTTHLSR

SEQ ID NO: 369 - protein, 7 aa, Artificial Sequence
  Description of Artificial Sequence Synthetic peptide
ESGHLKR

SEQ ID NO: 370 - protein, 7 aa, Artificial Sequence
  Description of Artificial Sequence Synthetic peptide
EKSHLKR

SEQ ID NO: 371 - protein, 7 aa, Artificial Sequence
  Description of Artificial Sequence Synthetic peptide
DKSHLPR

SEQ ID NO: 372 - protein, 7 aa, Artificial Sequence
  Description of Artificial Sequence Synthetic peptide
KNHSLNN

SEQ ID NO: 373 - protein, 7 aa, Artificial Sequence
  Description of Artificial Sequence Synthetic peptide
KNVSLTH

SEQ ID NO: 374 - protein, 7 aa, Artificial Sequence
  Description of Artificial Sequence Synthetic peptide
ESGHLKR

SEQ ID NO: 375 - protein, 7 aa, Artificial Sequence
  Description of Artificial Sequence Synthetic peptide
ESGHLKR

SEQ ID NO: 376 - protein, 7 aa, Artificial Sequence
  Description of Artificial Sequence Synthetic peptide
DGGHLTR

SEQ ID NO: 377 - protein, 7 aa, Artificial Sequence
  Description of Artificial Sequence Synthetic peptide
ENSKLNR

SEQ ID NO: 378 - protein, 7 aa, Artificial Sequence
  Description of Artificial Sequence Synthetic peptide
ESGHLRR

SEQ ID NO: 379 - protein, 7 aa, Artificial Sequence
  Description of Artificial Sequence Synthetic peptide
ESGHLKR

SEQ ID NO: 380 - protein, 7 aa, Artificial Sequence
  Description of Artificial Sequence Synthetic peptide
ESGHLKR

SEQ ID NO: 381 - protein, 7 aa, Artificial Sequence
  Description of Artificial Sequence Synthetic peptide
EKSHLTR

SEQ ID NO: 382 - protein, 7 aa, Artificial Sequence
  Description of Artificial Sequence Synthetic peptide
ESGHLKR

SEQ ID NO: 383 - protein, 7 aa, Artificial Sequence
  Description of Artificial Sequence Synthetic peptide
VDHHLRR

SEQ ID NO: 384 - protein, 7 aa, Artificial Sequence
  Description of Artificial Sequence Synthetic peptide
IRHHLKR

SEQ ID NO: 385 - protein, 7 aa, Artificial Sequence
  Description of Artificial Sequence Synthetic peptide
MGHHLKR

SEQ ID NO: 386 - protein, 7 aa, Artificial Sequence
  Description of Artificial Sequence Synthetic peptide
IRHHLKR

SEQ ID NO: 387 - protein, 7 aa, Artificial Sequence
  Description of Artificial Sequence Synthetic peptide
VKHGLGR

SEQ ID NO: 388 - protein, 7 aa, Artificial Sequence
  Description of Artificial Sequence Synthetic peptide
IRHHLKR

SEQ ID NO: 389 - protein, 7 aa, Artificial Sequence
  Description of Artificial Sequence Synthetic peptide
IRHHLKR

SEQ ID NO: 390 - protein, 7 aa, Artificial Sequence
  Description of Artificial Sequence Synthetic peptide
QPHHLPR

SEQ ID NO: 391 - protein, 7 aa, Artificial Sequence
  Description of Artificial Sequence Synthetic peptide
HGHRLKT

SEQ ID NO: 392 - protein, 7 aa, Artificial Sequence
  Description of Artificial Sequence Synthetic peptide
VDHHLRR

SEQ ID NO: 393 - protein, 7 aa, Artificial Sequence
  Description of Artificial Sequence Synthetic peptide
LTQGLRR

SEQ ID NO: 394 - protein, 7 aa, Artificial Sequence
  Description of Artificial Sequence Synthetic peptide
HGHRLKT

SEQ ID NO: 395 - protein, 7 aa, Artificial Sequence
  Description of Artificial Sequence Synthetic peptide
VKHGLTR

SEQ ID NO: 396 - protein, 7 aa, Artificial Sequence
  Description of Artificial Sequence Synthetic peptide
HGHRLKT

SEQ ID NO: 397 - protein, 7 aa, Artificial Sequence
  Description of Artificial Sequence Synthetic peptide
QPHHLPR

SEQ ID NO: 398 - protein, 7 aa, Artificial Sequence
  Description of Artificial Sequence Synthetic peptide
HGHRLKT

SEQ ID NO: 399 - protein, 7 aa, Artificial Sequence
  Description of Artificial Sequence Synthetic peptide
RRDNLLR

SEQ ID NO: 400 - protein, 7 aa, Artificial Sequence
  Description of Artificial Sequence Synthetic peptide
VHWNLMR

SEQ ID NO: 401 - protein, 7 aa, Artificial Sequence
  Description of Artificial Sequence Synthetic peptide
RVDNLPR

SEQ ID NO: 402 - protein, 7 aa, Artificial Sequence
  Description of Artificial Sequence Synthetic peptide
RGDNLKR

SEQ ID NO: 403 - protein, 7 aa, Artificial Sequence
  Description of Artificial Sequence Synthetic peptide
RVDNLPR

SEQ ID NO: 404 - protein, 7 aa, Artificial Sequence
  Description of Artificial Sequence Synthetic peptide
RRDNLNR

SEQ ID NO: 405 - protein, 7 aa, Artificial Sequence
  Description of Artificial Sequence Synthetic peptide
REDNLGR

SEQ ID NO: 406 - protein, 7 aa, Artificial Sequence
  Description of Artificial Sequence Synthetic peptide
REDNLPR

SEQ ID NO: 407 - protein, 7 aa, Artificial Sequence
  Description of Artificial Sequence Synthetic peptide
RRDNLLR

SEQ ID NO: 408 - protein, 7 aa, Artificial Sequence
  Description of Artificial Sequence Synthetic peptide
RGDNLNR

SEQ ID NO: 409 - protein, 7 aa, Artificial Sequence
  Description of Artificial Sequence Synthetic peptide
RVDNLPR

SEQ ID NO: 410 - protein, 7 aa, Artificial Sequence
  Description of Artificial Sequence Synthetic peptide
RQDNLQR

SEQ ID NO: 411 - protein, 7 aa, Artificial Sequence
  Description of Artificial Sequence Synthetic peptide
RRDNLLR

SEQ ID NO: 412 - protein, 7 aa, Artificial Sequence
  Description of Artificial Sequence Synthetic peptide
REDNLGR

SEQ ID NO: 413 - protein, 7 aa, Artificial Sequence
  Description of Artificial Sequence Synthetic peptide
RIDNLGR

SEQ ID NO: 414 - protein, 7 aa, Artificial Sequence
  Description of Artificial Sequence Synthetic peptide
RHDQLTR

SEQ ID NO: 415 - protein, 7 aa, Artificial Sequence
  Description of Artificial Sequence Synthetic peptide
RRDNLNR

SEQ ID NO: 416 - protein, 7 aa, Artificial Sequence
  Description of Artificial Sequence Synthetic peptide
RRDNLNR

SEQ ID NO: 417 - protein, 7 aa, Artificial Sequence
  Description of Artificial Sequence Synthetic peptide
QDGNLGR

SEQ ID NO: 418 - protein, 7 aa, Artificial Sequence
  Description of Artificial Sequence Synthetic peptide
LGENLRR

SEQ ID NO: 419 - protein, 7 aa, Artificial Sequence
  Description of Artificial Sequence Synthetic peptide
QTNNLTR

SEQ ID NO: 420 - protein, 7 aa, Artificial Sequence
  Description of Artificial Sequence Synthetic peptide
QDGNLTR

SEQ ID NO: 421 - protein, 7 aa, Artificial Sequence
  Description of Artificial Sequence Synthetic peptide
QRNNLGR

SEQ ID NO: 422 - protein, 7 aa, Artificial Sequence
  Description of Artificial Sequence Synthetic peptide
QTNNLNR

SEQ ID NO: 423 - protein, 7 aa, Artificial Sequence
  Description of Artificial Sequence Synthetic peptide
QSNNLNR

SEQ ID NO: 424 - protein, 7 aa, Artificial Sequence
  Description of Artificial Sequence Synthetic peptide
QRNNLGR

SEQ ID NO: 425 - protein, 7 aa, Artificial Sequence
  Description of Artificial Sequence Synthetic peptide
QTNNLGR

SEQ ID NO: 426 - protein, 7 aa, Artificial Sequence
  Description of Artificial Sequence Synthetic peptide
QRNNLGR

SEQ ID NO: 427 - protein, 7 aa, Artificial Sequence
  Description of Artificial Sequence Synthetic peptide
QHPNLTR

SEQ ID NO: 428 - protein, 7 aa, Artificial Sequence
  Description of Artificial Sequence Synthetic peptide
QRNNLGR

SEQ ID NO: 429 - protein, 7 aa, Artificial Sequence
  Description of Artificial Sequence Synthetic peptide
QTVNLDR

SEQ ID NO: 430 - protein, 7 aa, Artificial Sequence
  Description of Artificial Sequence Synthetic peptide
QRNNLGR

SEQ ID NO: 431 - protein, 7 aa, Artificial Sequence
  Description of Artificial Sequence Synthetic peptide
QHPNLTR

SEQ ID NO: 432 - protein, 7 aa, Artificial Sequence
  Description of Artificial Sequence Synthetic peptide
QTNNLGR

SEQ ID NO: 433 - protein, 7 aa, Artificial Sequence
  Description of Artificial Sequence Synthetic peptide
DPSNLQR

SEQ ID NO: 434 - protein, 7 aa, Artificial Sequence
  Description of Artificial Sequence Synthetic peptide
DPSNLRR

SEQ ID NO: 435 - protein, 7 aa, Artificial Sequence
  Description of Artificial Sequence Synthetic peptide
DPSNLRR

SEQ ID NO: 436 - protein, 7 aa, Artificial Sequence
  Description of Artificial Sequence Synthetic peptide
DPSNLRR

SEQ ID NO: 437 - protein, 7 aa, Artificial Sequence
  Description of Artificial Sequence Synthetic peptide
DPSNLRR

SEQ ID NO: 438 - protein, 7 aa, Artificial Sequence
  Description of Artificial Sequence Synthetic peptide
DMGNLGR

SEQ ID NO: 439 - protein, 7 aa, Artificial Sequence
  Description of Artificial Sequence Synthetic peptide
DQGNLIR

SEQ ID NO: 440 - protein, 7 aa, Artificial Sequence
  Description of Artificial Sequence Synthetic peptide
DPSNLRR

SEQ ID NO: 441 - protein, 7 aa, Artificial Sequence
  Description of Artificial Sequence Synthetic peptide
DPSNLRR

SEQ ID NO: 442 - protein, 7 aa, Artificial Sequence
  Description of Artificial Sequence Synthetic peptide
DPSNLRR

SEQ ID NO: 443 - protein, 7 aa, Artificial Sequence
  Description of Artificial Sequence Synthetic peptide
EGGNLMR

SEQ ID NO: 444 - protein, 7 aa, Artificial Sequence
  Description of Artificial Sequence Synthetic peptide
DPSNLRR

SEQ ID NO: 445 - protein, 7 aa, Artificial Sequence
  Description of Artificial Sequence Synthetic peptide
DPSNLRR

SEQ ID NO: 446 - protein, 7 aa, Artificial Sequence
  Description of Artificial Sequence Synthetic peptide
DMGNLGR

SEQ ID NO: 447 - protein, 7 aa, Artificial Sequence
  Description of Artificial Sequence Synthetic peptide
DPSNLRR

SEQ ID NO: 448 - protein, 7 aa, Artificial Sequence
  Description of Artificial Sequence Synthetic peptide
EGGNLMR

SEQ ID NO: 449 - protein, 7 aa, Artificial Sequence
  Description of Artificial Sequence Synthetic peptide
DPSNLRR

SEQ ID NO: 450 - protein, 7 aa, Artificial Sequence
  Description of Artificial Sequence Synthetic peptide
ISHNLAR

SEQ ID NO: 451 - protein, 7 aa, Artificial Sequence
  Description of Artificial Sequence Synthetic peptide
ISHNLAR

SEQ ID NO: 452 - protein, 7 aa, Artificial Sequence
  Description of Artificial Sequence Synthetic peptide
LSHNLAR

SEQ ID NO: 453 - protein, 7 aa, Artificial Sequence
  Description of Artificial Sequence Synthetic peptide
LNSNLAR

SEQ ID NO: 454 - protein, 7 aa, Artificial Sequence
  Description of Artificial Sequence Synthetic peptide
VGSNLTR

SEQ ID NO: 455 - protein, 7 aa, Artificial Sequence
  Description of Artificial Sequence Synthetic peptide
LGNNLKR

SEQ ID NO: 456 - protein, 7 aa, Artificial Sequence
  Description of Artificial Sequence Synthetic peptide
ISHNLAR

SEQ ID NO: 457 - protein, 7 aa, Artificial Sequence
  Description of Artificial Sequence Synthetic peptide
LNSNLAR

SEQ ID NO: 458 - protein, 7 aa, Artificial Sequence
  Description of Artificial Sequence Synthetic peptide
LNSNLAR

SEQ ID NO: 459 - protein, 7 aa, Artificial Sequence
  Description of Artificial Sequence Synthetic peptide
LSTNLTR

SEQ ID NO: 460 - protein, 7 aa, Artificial Sequence
  Description of Artificial Sequence Synthetic peptide
LTHNLRR

SEQ ID NO: 461 - protein, 7 aa, Artificial Sequence
  Description of Artificial Sequence Synthetic peptide
ISHNLAR

SEQ ID NO: 462 - protein, 7 aa, Artificial Sequence
  Description of Artificial Sequence Synthetic peptide
VGHNLSR

SEQ ID NO: 463 - protein, 7 aa, Artificial Sequence
  Description of Artificial Sequence Synthetic peptide
ISHNLAR

SEQ ID NO: 464 - protein, 7 aa, Artificial Sequence
  Description of Artificial Sequence Synthetic peptide
ISHNLAR

SEQ ID NO: 465 - protein, 7 aa, Artificial Sequence
  Description of Artificial Sequence Synthetic peptide
RTDSLPR

SEQ ID NO: 466 - protein, 7 aa, Artificial Sequence
  Description of Artificial Sequence Synthetic peptide
REDSLPR

SEQ ID NO: 467 - protein, 7 aa, Artificial Sequence
  Description of Artificial Sequence Synthetic peptide
RKDGLTR

SEQ ID NO: 468 - protein, 7 aa, Artificial Sequence
  Description of Artificial Sequence Synthetic peptide
RLDMLAR

SEQ ID NO: 469 - protein, 7 aa, Artificial Sequence
  Description of Artificial Sequence Synthetic peptide
RHAALLS

SEQ ID NO: 470 - protein, 7 aa, Artificial Sequence
  Description of Artificial Sequence Synthetic peptide
RLDMLAR

SEQ ID NO: 471 - protein, 7 aa, Artificial Sequence
  Description of Artificial Sequence Synthetic peptide
RRDGLTR

SEQ ID NO: 472 - protein, 7 aa, Artificial Sequence
  Description of Artificial Sequence Synthetic peptide
RVDDLGR

SEQ ID NO: 473 - protein, 7 aa, Artificial Sequence
  Description of Artificial Sequence Synthetic peptide
RVDDLGR

SEQ ID NO: 474 - protein, 7 aa, Artificial Sequence
  Description of Artificial Sequence Synthetic peptide
RLDMLAR

SEQ ID NO: 475 - protein, 7 aa, Artificial Sequence
  Description of Artificial Sequence Synthetic peptide
RVDDLGR

SEQ ID NO: 476 - protein, 7 aa, Artificial Sequence
  Description of Artificial Sequence Synthetic peptide
RLDMLAR

SEQ ID NO: 477 - protein, 7 aa, Artificial Sequence
  Description of Artificial Sequence Synthetic peptide
RPDGLAR

SEQ ID NO: 478 - protein, 7 aa, Artificial Sequence
  Description of Artificial Sequence Synthetic peptide
RRDDLTR

SEQ ID NO: 479 - protein, 7 aa, Artificial Sequence
  Description of Artificial Sequence Synthetic peptide
RTDLLRR

SEQ ID NO: 480 - protein, 7 aa, Artificial Sequence
  Description of Artificial Sequence Synthetic peptide
RVDDLGR

SEQ ID NO: 481 - protein, 7 aa, Artificial Sequence
  Description of Artificial Sequence Synthetic peptide
QTATLKR

SEQ ID NO: 482 - protein, 7 aa, Artificial Sequence
  Description of Artificial Sequence Synthetic peptide
QDVSLVR

SEQ ID NO: 483 - protein, 7 aa, Artificial Sequence
  Description of Artificial Sequence Synthetic peptide
QTNTLKR

SEQ ID NO: 484 - protein, 7 aa, Artificial Sequence
  Description of Artificial Sequence Synthetic peptide
QNGTLTR

SEQ ID NO: 485 - protein, 7 aa, Artificial Sequence
  Description of Artificial Sequence Synthetic peptide
QSNVLSR

SEQ ID NO: 486 - protein, 7 aa, Artificial Sequence
  Description of Artificial Sequence Synthetic peptide
QGNTLTR

SEQ ID NO: 487 - protein, 7 aa, Artificial Sequence
  Description of Artificial Sequence Synthetic peptide
QGNTLTR

SEQ ID NO: 488 - protein, 7 aa, Artificial Sequence
  Description of Artificial Sequence Synthetic peptide
QGNTLTR

SEQ ID NO: 489 - protein, 7 aa, Artificial Sequence
  Description of Artificial Sequence Synthetic peptide
QKGTLGR

SEQ ID NO: 490 - protein, 7 aa, Artificial Sequence
  Description of Artificial Sequence Synthetic peptide
QPNTLTR

SEQ ID NO: 491 - protein, 7 aa, Artificial Sequence
  Description of Artificial Sequence Synthetic peptide
QGGTLRR

SEQ ID NO: 492 - protein, 7 aa, Artificial Sequence
  Description of Artificial Sequence Synthetic peptide
QGGTLRR

SEQ ID NO: 493 - protein, 7 aa, Artificial Sequence
  Description of Artificial Sequence Synthetic peptide
QGGTLRR

SEQ ID NO: 494 - protein, 7 aa, Artificial Sequence
  Description of Artificial Sequence Synthetic peptide
QDNTLRR

SEQ ID NO: 495 - protein, 7 aa, Artificial Sequence
  Description of Artificial Sequence Synthetic peptide
QGGTLRR

SEQ ID NO: 496 - protein, 7 aa, Artificial Sequence
  Description of Artificial Sequence Synthetic peptide
QGNTLTR

SEQ ID NO: 497 - protein, 7 aa, Artificial Sequence
  Description of Artificial Sequence Synthetic peptide
EHRGLKR

SEQ ID NO: 498 - protein, 7 aa, Artificial Sequence
  Description of Artificial Sequence Synthetic peptide
DPSNLRR

SEQ ID NO: 499 - protein, 7 aa, Artificial Sequence
  Description of Artificial Sequence Synthetic peptide
DPSNLRR

SEQ ID NO: 500 - protein, 7 aa, Artificial Sequence
  Description of Artificial Sequence Synthetic peptide
DPSNLRR

SEQ ID NO: 501 - protein, 7 aa, Artificial Sequence
  Description of Artificial Sequence Synthetic peptide
DKSVLAR

SEQ ID NO: 502 - protein, 7 aa, Artificial Sequence
  Description of Artificial Sequence Synthetic peptide
EHRGLKR

SEQ ID NO: 503 - protein, 7 aa, Artificial Sequence
  Description of Artificial Sequence Synthetic peptide
DKSVLAR

SEQ ID NO: 504 - protein, 7 aa, Artificial Sequence
  Description of Artificial Sequence Synthetic peptide
DHSNLSR

SEQ ID NO: 505 - protein, 7 aa, Artificial Sequence
  Description of Artificial Sequence Synthetic peptide
ERRGLAR

SEQ ID NO: 506 - protein, 7 aa, Artificial Sequence
  Description of Artificial Sequence Synthetic peptide
DPSNLRR

SEQ ID NO: 507 - protein, 7 aa, Artificial Sequence
  Description of Artificial Sequence Synthetic peptide
ERRGLAR

SEQ ID NO: 508 - protein, 7 aa, Artificial Sequence
  Description of Artificial Sequence Synthetic peptide
DPSNLRR

SEQ ID NO: 509 - protein, 7 aa, Artificial Sequence
  Description of Artificial Sequence Synthetic peptide
ERRGLHR

SEQ ID NO: 510 - protein, 7 aa, Artificial Sequence
  Description of Artificial Sequence Synthetic peptide
DKSVLAR

SEQ ID NO: 511 - protein, 7 aa, Artificial Sequence
  Description of Artificial Sequence Synthetic peptide
DPSNLRR

SEQ ID NO: 512 - protein, 7 aa, Artificial Sequence
  Description of Artificial Sequence Synthetic peptide
DHSNLSR

SEQ ID NO: 513 - protein, 7 aa, Artificial Sequence
  Description of Artificial Sequence Synthetic peptide
VSNSLAR

SEQ ID NO: 514 - protein, 7 aa, Artificial Sequence
  Description of Artificial Sequence Synthetic peptide
LKHDLRR

SEQ ID NO: 515 - protein, 7 aa, Artificial Sequence
  Description of Artificial Sequence Synthetic peptide
EGSGLKR

SEQ ID NO: 516 - protein, 7 aa, Artificial Sequence
  Description of Artificial Sequence Synthetic peptide
VSNTLTR

SEQ ID NO: 517 - protein, 7 aa, Artificial Sequence
  Description of Artificial Sequence Synthetic peptide
VGNSLTR

SEQ ID NO: 518 - protein, 7 aa, Artificial Sequence
  Description of Artificial Sequence Synthetic peptide
VSNSLAR

SEQ ID NO: 519 - protein, 7 aa, Artificial Sequence
  Description of Artificial Sequence Synthetic peptide
LKHDLRR

SEQ ID NO: 520 - protein, 7 aa, Artificial Sequence
  Description of Artificial Sequence Synthetic peptide
LKHDLRR

SEQ ID NO: 521 - protein, 7 aa, Artificial Sequence
  Description of Artificial Sequence Synthetic peptide
LKHDLRR

SEQ ID NO: 522 - protein, 7 aa, Artificial Sequence
  Description of Artificial Sequence Synthetic peptide
QRSDLHR

SEQ ID NO: 523 - protein, 7 aa, Artificial Sequence
  Description of Artificial Sequence Synthetic peptide
LRASLRR

SEQ ID NO: 524 - protein, 7 aa, Artificial Sequence
  Description of Artificial Sequence Synthetic peptide
VGASLKR

SEQ ID NO: 525 - protein, 7 aa, Artificial Sequence
  Description of Artificial Sequence Synthetic peptide
VKNTLTR

SEQ ID NO: 526 - protein, 7 aa, Artificial Sequence
  Description of Artificial Sequence Synthetic peptide
VSNSLAR

SEQ ID NO: 527 - protein, 7 aa, Artificial Sequence
  Description of Artificial Sequence Synthetic peptide
VSNTLTR

SEQ ID NO: 528 - protein, 7 aa, Artificial Sequence
  Description of Artificial Sequence Synthetic peptide
RPDALPR

SEQ ID NO: 529 - protein, 7 aa, Artificial Sequence
  Description of Artificial Sequence Synthetic peptide
RNTALQH

SEQ ID NO: 530 - protein, 7 aa, Artificial Sequence
  Description of Artificial Sequence Synthetic peptide
RKDALHV

SEQ ID NO: 531 - protein, 7 aa, Artificial Sequence
  Description of Artificial Sequence Synthetic peptide
RKDALHV

SEQ ID NO: 532 - protein, 7 aa, Artificial Sequence
  Description of Artificial Sequence Synthetic peptide
RNVNLVT

SEQ ID NO: 533 - protein, 7 aa, Artificial Sequence
  Description of Artificial Sequence Synthetic peptide
RNVALGN

SEQ ID NO: 534 - protein, 7 aa, Artificial Sequence
  Description of Artificial Sequence Synthetic peptide
RGDALAR

SEQ ID NO: 535 - protein, 7 aa, Artificial Sequence
  Description of Artificial Sequence Synthetic peptide
RRAALGP

SEQ ID NO: 536 - protein, 7 aa, Artificial Sequence
  Description of Artificial Sequence Synthetic peptide
RKTALNR

SEQ ID NO: 537 - protein, 7 aa, Artificial Sequence
  Description of Artificial Sequence Synthetic peptide
RKDALHV

SEQ ID NO: 538 - protein, 7 aa, Artificial Sequence
  Description of Artificial Sequence Synthetic peptide
RHTSLTR

SEQ ID NO: 539 - protein, 7 aa, Artificial Sequence
  Description of Artificial Sequence Synthetic peptide
RPDALPR

SEQ ID NO: 540 - protein, 7 aa, Artificial Sequence
  Description of Artificial Sequence Synthetic peptide
RKDALHV

SEQ ID NO: 541 - protein, 7 aa, Artificial Sequence
  Description of Artificial Sequence Synthetic peptide
RTVALNR

SEQ ID NO: 542 - protein, 7 aa, Artificial Sequence
  Description of Artificial Sequence Synthetic peptide
RHTSLTR

SEQ ID NO: 543 - protein, 7 aa, Artificial Sequence
  Description of Artificial Sequence Synthetic peptide
RNTALQH

SEQ ID NO: 544 - protein, 7 aa, Artificial Sequence
  Description of Artificial Sequence Synthetic peptide
QSTSLQR

SEQ ID NO: 545 - protein, 7 aa, Artificial Sequence
  Description of Artificial Sequence Synthetic peptide
QSTSLQR

SEQ ID NO: 546 - protein, 7 aa, Artificial Sequence
  Description of Artificial Sequence Synthetic peptide
QTQSLQR

SEQ ID NO: 547 - protein, 7 aa, Artificial Sequence
  Description of Artificial Sequence Synthetic peptide
QSTSLQR

SEQ ID NO: 548 - protein, 7 aa, Artificial Sequence
  Description of Artificial Sequence Synthetic peptide
QMNALQR

SEQ ID NO: 549 - protein, 7 aa, Artificial Sequence
  Description of Artificial Sequence Synthetic peptide
QSTSLQR

SEQ ID NO: 550 - protein, 7 aa, Artificial Sequence
  Description of Artificial Sequence Synthetic peptide
QSTSLQR

SEQ ID NO: 551 - protein, 7 aa, Artificial Sequence
  Description of Artificial Sequence Synthetic peptide
QSTSLQR

SEQ ID NO: 552 - protein, 7 aa, Artificial Sequence
  Description of Artificial Sequence Synthetic peptide
QGGALQR

SEQ ID NO: 553 - protein, 7 aa, Artificial Sequence
  Description of Artificial Sequence Synthetic peptide
QKVSLKR

SEQ ID NO: 554 - protein, 7 aa, Artificial Sequence
  Description of Artificial Sequence Synthetic peptide
QSTSLQR

SEQ ID NO: 555 - protein, 7 aa, Artificial Sequence
  Description of Artificial Sequence Synthetic peptide
QSGTLTR

SEQ ID NO: 556 - protein, 7 aa, Artificial Sequence
  Description of Artificial Sequence Synthetic peptide
QSTSLQR

SEQ ID NO: 557 - protein, 7 aa, Artificial Sequence
  Description of Artificial Sequence Synthetic peptide
QGTSLAR

SEQ ID NO: 558 - protein, 7 aa, Artificial Sequence
  Description of Artificial Sequence Synthetic peptide
QSTSLQR

SEQ ID NO: 559 - protein, 7 aa, Artificial Sequence
  Description of Artificial Sequence Synthetic peptide
QSTSLQR

SEQ ID NO: 560 - protein, 7 aa, Artificial Sequence
  Description of Artificial Sequence Synthetic peptide
DPTSLNR

SEQ ID NO: 561 - protein, 7 aa, Artificial Sequence
  Description of Artificial Sequence Synthetic peptide
DPTSLNR

SEQ ID NO: 562 - protein, 7 aa, Artificial Sequence
  Description of Artificial Sequence Synthetic peptide
DPTSLNR

SEQ ID NO: 563 - protein, 7 aa, Artificial Sequence
  Description of Artificial Sequence Synthetic peptide
DRSSLRR

SEQ ID NO: 564 - protein, 7 aa, Artificial Sequence
  Description of Artificial Sequence Synthetic peptide
DRTPLNR

SEQ ID NO: 565 - protein, 7 aa, Artificial Sequence
  Description of Artificial Sequence Synthetic peptide
DATQLVR

SEQ ID NO: 566 - protein, 7 aa, Artificial Sequence
  Description of Artificial Sequence Synthetic peptide
DRSSLRR

SEQ ID NO: 567 - protein, 7 aa, Artificial Sequence
  Description of Artificial Sequence Synthetic peptide
DRTSLAR

SEQ ID NO: 568 - protein, 7 aa, Artificial Sequence
  Description of Artificial Sequence Synthetic peptide
ESGALRR

SEQ ID NO: 569 - protein, 7 aa, Artificial Sequence
  Description of Artificial Sequence Synthetic peptide
EGGALRR

SEQ ID NO: 570 - protein, 7 aa, Artificial Sequence
  Description of Artificial Sequence Synthetic peptide
DRSSLRR

SEQ ID NO: 571 - protein, 7 aa, Artificial Sequence
  Description of Artificial Sequence Synthetic peptide
DRSSLRR

SEQ ID NO: 572 - protein, 7 aa, Artificial Sequence
  Description of Artificial Sequence Synthetic peptide
DRTPLNR

SEQ ID NO: 573 - protein, 7 aa, Artificial Sequence
  Description of Artificial Sequence Synthetic peptide
DPTSLNR

SEQ ID NO: 574 - protein, 7 aa, Artificial Sequence
  Description of Artificial Sequence Synthetic peptide
DRTPLQR

SEQ ID NO: 575 - protein, 7 aa, Artificial Sequence
  Description of Artificial Sequence Synthetic peptide
DRSSLRR

SEQ ID NO: 576 - protein, 7 aa, Artificial Sequence
  Description of Artificial Sequence Synthetic peptide
INHSLRR

SEQ ID NO: 577 - protein, 7 aa, Artificial Sequence
  Description of Artificial Sequence Synthetic peptide
HHNSLTR

SEQ ID NO: 578 - protein, 7 aa, Artificial Sequence
  Description of Artificial Sequence Synthetic peptide
IRTSLKR

SEQ ID NO: 579 - protein, 7 aa, Artificial Sequence
  Description of Artificial Sequence Synthetic peptide
HHNSLTR

SEQ ID NO: 580 - protein, 7 aa, Artificial Sequence
  Description of Artificial Sequence Synthetic peptide
VGGSLNR

SEQ ID NO: 581 - protein, 7 aa, Artificial Sequence
  Description of Artificial Sequence Synthetic peptide
HHNSLTR

SEQ ID NO: 582 - protein, 7 aa, Artificial Sequence
  Description of Artificial Sequence Synthetic peptide
HHNSLTR

SEQ ID NO: 583 - protein, 7 aa, Artificial Sequence
  Description of Artificial Sequence Synthetic peptide
IRTSLKR

SEQ ID NO: 584 - protein, 7 aa, Artificial Sequence
  Description of Artificial Sequence Synthetic peptide
TRHSLGR

SEQ ID NO: 585 - protein, 7 aa, Artificial Sequence
  Description of Artificial Sequence Synthetic peptide
IRTSLKR

SEQ ID NO: 586 - protein, 7 aa, Artificial Sequence
  Description of Artificial Sequence Synthetic peptide
IRTSLKR

SEQ ID NO: 587 - protein, 7 aa, Artificial Sequence
  Description of Artificial Sequence Synthetic peptide
INHSLRR

SEQ ID NO: 588 - protein, 7 aa, Artificial Sequence
  Description of Artificial Sequence Synthetic peptide
HHNSLTR

SEQ ID NO: 589 - protein, 7 aa, Artificial Sequence
  Description of Artificial Sequence Synthetic peptide
HHNSLTR

SEQ ID NO: 590 - protein, 7 aa, Artificial Sequence
  Description of Artificial Sequence Synthetic peptide
IRTSLKR

SEQ ID NO: 591 - protein, 7 aa, Artificial Sequence
  Description of Artificial Sequence Synthetic peptide
RMDHLAG

SEQ ID NO: 592 - protein, 7 aa, Artificial Sequence
  Description of Artificial Sequence Synthetic peptide
RSDHLSL

SEQ ID NO: 593 - protein, 7 aa, Artificial Sequence
  Description of Artificial Sequence Synthetic peptide
RTESLHI

SEQ ID NO: 594 - protein, 7 aa, Artificial Sequence
  Description of Artificial Sequence Synthetic peptide
RSDHLSL

SEQ ID NO: 595 - protein, 7 aa, Artificial Sequence
  Description of Artificial Sequence Synthetic peptide
RMDHLAG

SEQ ID NO: 596 - protein, 7 aa, Artificial Sequence
  Description of Artificial Sequence Synthetic peptide
RSDHLSL

SEQ ID NO: 597 - protein, 7 aa, Artificial Sequence
  Description of Artificial Sequence Synthetic peptide
RSDHLSL

SEQ ID NO: 598 - protein, 7 aa, Artificial Sequence
  Description of Artificial Sequence Synthetic peptide
RSDHLSL

SEQ ID NO: 599 - protein, 7 aa, Artificial Sequence
  Description of Artificial Sequence Synthetic peptide
RSDHLSL

SEQ ID NO: 600 - protein, 7 aa, Artificial Sequence
  Description of Artificial Sequence Synthetic peptide
RSDHLSL

SEQ ID NO: 601 - protein, 7 aa, Artificial Sequence
  Description of Artificial Sequence Synthetic peptide
RSDHLSL

SEQ ID NO: 602 - protein, 7 aa, Artificial Sequence
  Description of Artificial Sequence Synthetic peptide
RSDHLSL

SEQ ID NO: 603 - protein, 7 aa, Artificial Sequence
  Description of Artificial Sequence Synthetic peptide
RRDHLSL

SEQ ID NO: 604 - protein, 7 aa, Artificial Sequence
  Description of Artificial Sequence Synthetic peptide
RSDHLSL

SEQ ID NO: 605 - protein, 7 aa, Artificial Sequence
  Description of Artificial Sequence Synthetic peptide
RSDHLSL

SEQ ID NO: 606 - protein, 7 aa, Artificial Sequence
  Description of Artificial Sequence Synthetic peptide
RSDHLSL

SEQ ID NO: 607 - protein, 7 aa, Artificial Sequence
  Description of Artificial Sequence Synthetic peptide
ANRTLVH

SEQ ID NO: 608 - protein, 7 aa, Artificial Sequence
  Description of Artificial Sequence Synthetic peptide
QRRSLGH

SEQ ID NO: 609 - protein, 7 aa, Artificial Sequence
  Description of Artificial Sequence Synthetic peptide
ANRTLVH

SEQ ID NO: 610 - protein, 7 aa, Artificial Sequence
  Description of Artificial Sequence Synthetic peptide
ANRTLVH

SEQ ID NO: 611 - protein, 7 aa, Artificial Sequence
  Description of Artificial Sequence Synthetic peptide
QARSLRA

SEQ ID NO: 612 - protein, 7 aa, Artificial Sequence
  Description of Artificial Sequence Synthetic peptide
QRRSLGH

SEQ ID NO: 613 - protein, 7 aa, Artificial Sequence
  Description of Artificial Sequence Synthetic peptide
QGRSLRA

SEQ ID NO: 614 - protein, 7 aa, Artificial Sequence
  Description of Artificial Sequence Synthetic peptide
QQRSLKN

SEQ ID NO: 615 - protein, 7 aa, Artificial Sequence
  Description of Artificial Sequence Synthetic peptide
ANRTLVH

SEQ ID NO: 616 - protein, 7 aa, Artificial Sequence
  Description of Artificial Sequence Synthetic peptide
ANRTLVH

SEQ ID NO: 617 - protein, 7 aa, Artificial Sequence
  Description of Artificial Sequence Synthetic peptide
QNRSLAH

SEQ ID NO: 618 - protein, 7 aa, Artificial Sequence
  Description of Artificial Sequence Synthetic peptide
QRHGLSS

SEQ ID NO: 619 - protein, 7 aa, Artificial Sequence
  Description of Artificial Sequence Synthetic peptide
QPHGLAH

SEQ ID NO: 620 - protein, 7 aa, Artificial Sequence
  Description of Artificial Sequence Synthetic peptide
QAHGLTG

SEQ ID NO: 621 - protein, 7 aa, Artificial Sequence
  Description of Artificial Sequence Synthetic peptide
QPHGLAH

SEQ ID NO: 622 - protein, 7 aa, Artificial Sequence
  Description of Artificial Sequence Synthetic peptide
QQHGLRH

SEQ ID NO: 623 - protein, 7 aa, Artificial Sequence
  Description of Artificial Sequence Synthetic peptide
QPHGLTA

SEQ ID NO: 624 - protein, 7 aa, Artificial Sequence
  Description of Artificial Sequence Synthetic peptide
QPHGLRA

SEQ ID NO: 625 - protein, 7 aa, Artificial Sequence
  Description of Artificial Sequence Synthetic peptide
QPHGLAH

SEQ ID NO: 626 - protein, 7 aa, Artificial Sequence
  Description of Artificial Sequence Synthetic peptide
QPHGLGA

SEQ ID NO: 627 - protein, 7 aa, Artificial Sequence
  Description of Artificial Sequence Synthetic peptide
QAHGLTA

SEQ ID NO: 628 - protein, 7 aa, Artificial Sequence
  Description of Artificial Sequence Synthetic peptide
QAHGLTA

SEQ ID NO: 629 - protein, 7 aa, Artificial Sequence
  Description of Artificial Sequence Synthetic peptide
QPHGLRH

SEQ ID NO: 630 - protein, 7 aa, Artificial Sequence
  Description of Artificial Sequence Synthetic peptide
QRHGLSS

SEQ ID NO: 631 - protein, 7 aa, Artificial Sequence
  Description of Artificial Sequence Synthetic peptide
QPHGLAH

SEQ ID NO: 632 - protein, 7 aa, Artificial Sequence
  Description of Artificial Sequence Synthetic peptide
QPHGLAH

SEQ ID NO: 633 - protein, 7 aa, Artificial Sequence
  Description of Artificial Sequence Synthetic peptide
QPHGLAH

SEQ ID NO: 634 - protein, 7 aa, Artificial Sequence
  Description of Artificial Sequence Synthetic peptide
RPESLAP

SEQ ID NO: 635 - protein, 7 aa, Artificial Sequence
  Description of Artificial Sequence Synthetic peptide
RPEGLST

SEQ ID NO: 636 - protein, 7 aa, Artificial Sequence
  Description of Artificial Sequence Synthetic peptide
RRDNLPK

SEQ ID NO: 637 - protein, 7 aa, Artificial Sequence
  Description of Artificial Sequence Synthetic peptide
RPESLRP

SEQ ID NO: 638 - protein, 7 aa, Artificial Sequence
  Description of Artificial Sequence Synthetic peptide
RRDHLSL

SEQ ID NO: 639 - protein, 7 aa, Artificial Sequence
  Description of Artificial Sequence Synthetic peptide
RKTGLLI

SEQ ID NO: 640 - protein, 7 aa, Artificial Sequence
  Description of Artificial Sequence Synthetic peptide
RIDHLVP

SEQ ID NO: 641 - protein, 7 aa, Artificial Sequence
  Description of Artificial Sequence Synthetic peptide
RRDHLSP

SEQ ID NO: 642 - protein, 7 aa, Artificial Sequence
  Description of Artificial Sequence Synthetic peptide
RRDGLAG

SEQ ID NO: 643 - protein, 7 aa, Artificial Sequence
  Description of Artificial Sequence Synthetic peptide
RHDGLAG

SEQ ID NO: 644 - protein, 7 aa, Artificial Sequence
  Description of Artificial Sequence Synthetic peptide
RRDNLPK

SEQ ID NO: 645 - protein, 7 aa, Artificial Sequence
  Description of Artificial Sequence Synthetic peptide
RLDGLAG

SEQ ID NO: 646 - protein, 7 aa, Artificial Sequence
  Description of Artificial Sequence Synthetic peptide
RPESLAP

SEQ ID NO: 647 - protein, 7 aa, Artificial Sequence
  Description of Artificial Sequence Synthetic peptide
QQGNLRN

SEQ ID NO: 648 - protein, 7 aa, Artificial Sequence
  Description of Artificial Sequence Synthetic peptide
QRGNLNM

SEQ ID NO: 649 - protein, 7 aa, Artificial Sequence
  Description of Artificial Sequence Synthetic peptide
QGGNLTL

SEQ ID NO: 650 - protein, 7 aa, Artificial Sequence
  Description of Artificial Sequence Synthetic peptide
QGGNLAL

SEQ ID NO: 651 - protein, 7 aa, Artificial Sequence
  Description of Artificial Sequence Synthetic peptide
QSGNLHT

SEQ ID NO: 652 - protein, 7 aa, Artificial Sequence
  Description of Artificial Sequence Synthetic peptide
QQGNLQL

SEQ ID NO: 653 - protein, 7 aa, Artificial Sequence
  Description of Artificial Sequence Synthetic peptide
QGGNLAL

SEQ ID NO: 654 - protein, 7 aa, Artificial Sequence
  Description of Artificial Sequence Synthetic peptide
QSGNLHT

SEQ ID NO: 655 - protein, 7 aa, Artificial Sequence
  Description of Artificial Sequence Synthetic peptide
QRGNLNM

SEQ ID NO: 656 - protein, 7 aa, Artificial Sequence
  Description of Artificial Sequence Synthetic peptide
QRGNLNM

SEQ ID NO: 657 - protein, 7 aa, Artificial Sequence
  Description of Artificial Sequence Synthetic peptide
QQGNLQL

SEQ ID NO: 658 - protein, 7 aa, Artificial Sequence
  Description of Artificial Sequence Synthetic peptide
QQGNLGL

SEQ ID NO: 659 - protein, 7 aa, Artificial Sequence
  Description of Artificial Sequence Synthetic peptide
QQGNLQL

SEQ ID NO: 660 - protein, 7 aa, Artificial Sequence
  Description of Artificial Sequence Synthetic peptide
RMDSLGG

SEQ ID NO: 661 - protein, 7 aa, Artificial Sequence
  Description of Artificial Sequence Synthetic peptide
RGDSLKK

SEQ ID NO: 662 - protein, 7 aa, Artificial Sequence
  Description of Artificial Sequence Synthetic peptide
RSDGLRG

SEQ ID NO: 663 - protein, 7 aa, Artificial Sequence
  Description of Artificial Sequence Synthetic peptide
RADGLQL

SEQ ID NO: 664 - protein, 7 aa, Artificial Sequence
  Description of Artificial Sequence Synthetic peptide
RTDSLQP

SEQ ID NO: 665 - protein, 7 aa, Artificial Sequence
  Description of Artificial Sequence Synthetic peptide
RRDGLSG

SEQ ID NO: 666 - protein, 7 aa, Artificial Sequence
  Description of Artificial Sequence Synthetic peptide
RADGLQL

SEQ ID NO: 667 - protein, 7 aa, Artificial Sequence
  Description of Artificial Sequence Synthetic peptide
RSDTLPA

SEQ ID NO: 668 - protein, 7 aa, Artificial Sequence
  Description of Artificial Sequence Synthetic peptide
RSDTLPL

SEQ ID NO: 669 - protein, 7 aa, Artificial Sequence
  Description of Artificial Sequence Synthetic peptide
RADGLQL

SEQ ID NO: 670 - protein, 7 aa, Artificial Sequence
  Description of Artificial Sequence Synthetic peptide
RRDGLSG

SEQ ID NO: 671 - protein, 7 aa, Artificial Sequence
  Description of Artificial Sequence Synthetic peptide
RSDGLRG

SEQ ID NO: 672 - protein, 7 aa, Artificial Sequence
  Description of Artificial Sequence Synthetic peptide
RADGLQL

SEQ ID NO: 673 - protein, 7 aa, Artificial Sequence
  Description of Artificial Sequence Synthetic peptide
RRDGLSG

SEQ ID NO: 674 - protein, 7 aa, Artificial Sequence
  Description of Artificial Sequence Synthetic peptide
RGDSLKK

SEQ ID NO: 675 - protein, 7 aa, Artificial Sequence
  Description of Artificial Sequence Synthetic peptide
RADGLQL

SEQ ID NO: 676 - protein, 7 aa, Artificial Sequence
  Description of Artificial Sequence Synthetic peptide
DKRSLPH

SEQ ID NO: 677 - protein, 7 aa, Artificial Sequence
  Description of Artificial Sequence Synthetic peptide
QRNTLKG

SEQ ID NO: 678 - protein, 7 aa, Artificial Sequence
  Description of Artificial Sequence Synthetic peptide
QRNTLKG

SEQ ID NO: 679 - protein, 7 aa, Artificial Sequence
  Description of Artificial Sequence Synthetic peptide
QQTGLNV

SEQ ID NO: 680 - protein, 7 aa, Artificial Sequence
  Description of Artificial Sequence Synthetic peptide
QRNALRG

SEQ ID NO: 681 - protein, 7 aa, Artificial Sequence
  Description of Artificial Sequence Synthetic peptide
QRNALSG

SEQ ID NO: 682 - DNA, 11 bp, Artificial Sequence
  Description of Artificial Sequence Synthetic oligonucleotide
agatgaagagg

SEQ ID NO: 683 - DNA, 11 bp, Artificial Sequence
  Description of Artificial Sequence Synthetic oligonucleotide
agatgaagagg

SEQ ID NO: 684 - DNA, 11 bp, Artificial Sequence
  Description of Artificial Sequence Synthetic oligonucleotide
tgatgaagagc

SEQ ID NO: 685 - DNA, 11 bp, Artificial Sequence
  Description of Artificial Sequence Synthetic oligonucleotide
tgatgaagagc

SEQ ID NO: 686 - DNA, 11 bp, Artificial Sequence
  Description of Artificial Sequence Synthetic oligonucleotide
agaagatgtgg

SEQ ID NO: 687 - DNA, 11 bp, Artificial Sequence
  Description of Artificial Sequence Synthetic oligonucleotide
tgactgggtgc

SEQ ID NO: 688 - DNA, 11 bp, Artificial Sequence
  Description of Artificial Sequence Synthetic oligonucleotide
agaagcaaaca

SEQ ID NO: 689 - DNA, 11 bp, Artificial Sequence
  Description of Artificial Sequence Synthetic oligonucleotide
ctctgctgctt

SEQ ID NO: 690 - DNA, 11 bp, Artificial Sequence
  Description of Artificial Sequence Synthetic oligonucleotide
cgctgagtctg

SEQ ID NO: 691 - DNA, 11 bp, Artificial Sequence
  Description of Artificial Sequence Synthetic oligonucleotide
gtctgctggag

SEQ ID NO: 692 - DNA, 11 bp, Artificial Sequence
  Description of Artificial Sequence Synthetic oligonucleotide
ggcggccgtca

SEQ ID NO: 693 - DNA, 11 bp, Artificial Sequence
  Description of Artificial Sequence Synthetic oligonucleotide
ggcggccgtca

SEQ ID NO: 694 - DNA, 11 bp, Artificial Sequence
  Description of Artificial Sequence Synthetic oligonucleotide
ggtctgaggaa

SEQ ID NO: 695 - DNA, 11 bp, Artificial Sequence
  Description of Artificial Sequence Synthetic oligonucleotide
atctgctgagg

SEQ ID NO: 696 - DNA, 11 bp, Artificial Sequence
  Description of Artificial Sequence Synthetic oligonucleotide
ggccgctggtc

SEQ ID NO: 697 - DNA, 11 bp, Artificial Sequence
  Description of Artificial Sequence Synthetic oligonucleotide
tgcagcagcat

SEQ ID NO: 698 - DNA, 11 bp, Artificial Sequence
  Description of Artificial Sequence Synthetic oligonucleotide
tgctgcaggaa

SEQ ID NO: 699 - DNA, 11 bp, Artificial Sequence
  Description of Artificial Sequence Synthetic oligonucleotide
agaaggaggtg

SEQ ID NO: 700 - DNA, 11 bp, Artificial Sequence
  Description of Artificial Sequence Synthetic oligonucleotide
tgctgctggca

SEQ ID NO: 701 - DNA, 11 bp, Artificial Sequence
  Description of Artificial Sequence Synthetic oligonucleotide
tgctgatggag

SEQ ID NO: 702 - DNA, 11 bp, Artificial Sequence
  Description of Artificial Sequence Synthetic oligonucleotide
agtcgatgtgg

SEQ ID NO: 703 - DNA, 11 bp, Artificial Sequence
  Description of Artificial Sequence Synthetic oligonucleotide
ggcagaagacc

SEQ ID NO: 704 - DNA, 11 bp, Artificial Sequence
  Description of Artificial Sequence Synthetic oligonucleotide
agctgctggtc

SEQ ID NO: 705 - DNA, 11 bp, Artificial Sequence
  Description of Artificial Sequence Synthetic oligonucleotide
tgacgctgagg

SEQ ID NO: 706 - DNA, 11 bp, Artificial Sequence
  Description of Artificial Sequence Synthetic oligonucleotide
tgctggaggtg

SEQ ID NO: 707 - DNA, 11 bp, Artificial Sequence
  Description of Artificial Sequence Synthetic oligonucleotide
agatgaagatg

SEQ ID NO: 708 - DNA, 11 bp, Artificial Sequence
  Description of Artificial Sequence Synthetic oligonucleotide
tgacgccgcgc

SEQ ID NO: 709 - DNA, 11 bp, Artificial Sequence
  Description of Artificial Sequence Synthetic oligonucleotide
tgacgccgcgc

SEQ ID NO: 710 - protein, 7 aa, Artificial Sequence
  Description of Artificial Sequence Synthetic peptide
KHSNLTR

SEQ ID NO: 711 - protein, 7 aa, Artificial Sequence
  Description of Artificial Sequence Synthetic peptide
KHSNLTR

SEQ ID NO: 712 - protein, 7 aa, Artificial Sequence
  Description of Artificial Sequence Synthetic peptide
KHSNLTR

SEQ ID NO: 713 - protein, 7 aa, Artificial Sequence
  Description of Artificial Sequence Synthetic peptide
KHSNLTR

SEQ ID NO: 714 - protein, 7 aa, Artificial Sequence
  Description of Artificial Sequence Synthetic peptide
RKSVLHN

SEQ ID NO: 715 - protein, 7 aa, Artificial Sequence
  Description of Artificial Sequence Synthetic peptide
RNFILQR

SEQ ID NO: 716 - protein, 7 aa, Artificial Sequence
  Description of Artificial Sequence Synthetic peptide
GHTALRN

SEQ ID NO: 717 - protein, 7 aa, Artificial Sequence
  Description of Artificial Sequence Synthetic peptide
TKQVLDR

SEQ ID NO: 718 - protein, 7 aa, Artificial Sequence
  Description of Artificial Sequence Synthetic peptide
SKPNLKM

SEQ ID NO: 719 - protein, 7 aa, Artificial Sequence
  Description of Artificial Sequence Synthetic peptide
QKQHLMV

SEQ ID NO: 720 - protein, 7 aa, Artificial Sequence
  Description of Artificial Sequence Synthetic peptide
TRAVLAR

SEQ ID NO: 721 - protein, 7 aa, Artificial Sequence
  Description of Artificial Sequence Synthetic peptide
TRAVLAR

SEQ ID NO: 722 - protein, 7 aa, Artificial Sequence
  Description of Artificial Sequence Synthetic peptide
RQDRLDR

SEQ ID NO: 723 - protein, 7 aa, Artificial Sequence
  Description of Artificial Sequence Synthetic peptide
KHSNLTR

SEQ ID NO: 724 - protein, 7 aa, Artificial Sequence
  Description of Artificial Sequence Synthetic peptide
LRHHLEA

SEQ ID NO: 725 - protein, 7 aa, Artificial Sequence
  Description of Artificial Sequence Synthetic peptide
QRGTLNR

SEQ ID NO: 726 - protein, 7 aa, Artificial Sequence
  Description of Artificial Sequence Synthetic peptide
QQAHLVR

SEQ ID NO: 727 - protein, 7 aa, Artificial Sequence
  Description of Artificial Sequence Synthetic peptide
IPNHLAR

SEQ ID NO: 728 - protein, 7 aa, Artificial Sequence
  Description of Artificial Sequence Synthetic peptide
APSKLKR

SEQ ID NO: 729 - protein, 7 aa, Artificial Sequence
  Description of Artificial Sequence Synthetic peptide
DKTKLRV

SEQ ID NO: 730 - protein, 7 aa, Artificial Sequence
  Description of Artificial Sequence Synthetic peptide
RKSVLHN

SEQ ID NO: 731 - protein, 7 aa, Artificial Sequence
  Description of Artificial Sequence Synthetic peptide
DEANLRR

SEQ ID NO: 732 - protein, 7 aa, Artificial Sequence
  Description of Artificial Sequence Synthetic peptide
TTTKLAI

SEQ ID NO: 733 - protein, 7 aa, Artificial Sequence
  Description of Artificial Sequence Synthetic peptide
KHSNLTR

SEQ ID NO: 734 - protein, 7 aa, Artificial Sequence
  Description of Artificial Sequence Synthetic peptide
IPNHLAR

SEQ ID NO: 735 - protein, 7 aa, Artificial Sequence
  Description of Artificial Sequence Synthetic peptide
SNQALGV

SEQ ID NO: 736 - protein, 7 aa, Artificial Sequence
  Description of Artificial Sequence Synthetic peptide
RLRDLPR

SEQ ID NO: 737 - protein, 7 aa, Artificial Sequence
  Description of Artificial Sequence Synthetic peptide
RRNSLKR

SEQ ID NO: 738 - protein, 7 aa, Artificial Sequence
  Description of Artificial Sequence Synthetic peptide
QKVNLAR

SEQ ID NO: 739 - protein, 7 aa, Artificial Sequence
  Description of Artificial Sequence Synthetic peptide
QQTNLAR

SEQ ID NO: 740 - protein, 7 aa, Artificial Sequence
  Description of Artificial Sequence Synthetic peptide
QKVNLAR

SEQ ID NO: 741 - protein, 7 aa, Artificial Sequence
  Description of Artificial Sequence Synthetic peptide
QQTNLAR

SEQ ID NO: 742 - protein, 7 aa, Artificial Sequence
  Description of Artificial Sequence Synthetic peptide
VRHNLTR

SEQ ID NO: 743 - protein, 7 aa, Artificial Sequence
  Description of Artificial Sequence Synthetic peptide
RREHLTI

SEQ ID NO: 744 - protein, 7 aa, Artificial Sequence
  Description of Artificial Sequence Synthetic peptide
QSGTLKR

SEQ ID NO: 745 - protein, 7 aa, Artificial Sequence
  Description of Artificial Sequence Synthetic peptide
VKHSLQR

SEQ ID NO: 746 - protein, 7 aa, Artificial Sequence
  Description of Artificial Sequence Synthetic peptide
RGDNLGR

SEQ ID NO: 747 - protein, 7 aa, Artificial Sequence
  Description of Artificial Sequence Synthetic peptide
VKHSLQR

SEQ ID NO: 748 - protein, 7 aa, Artificial Sequence
  Description of Artificial Sequence Synthetic peptide
DSSVLRR

SEQ ID NO: 749 - protein, 7 aa, Artificial Sequence
  Description of Artificial Sequence Synthetic peptide
DSSVLRR

SEQ ID NO: 750 - protein, 7 aa, Artificial Sequence
  Description of Artificial Sequence Synthetic peptide
QKEHLAG

SEQ ID NO: 751 - protein, 7 aa, Artificial Sequence
  Description of Artificial Sequence Synthetic peptide
QRSDLTR

SEQ ID NO: 752 - protein, 7 aa, Artificial Sequence
  Description of Artificial Sequence Synthetic peptide
VAHSLKR

SEQ ID NO: 753 - protein, 7 aa, Artificial Sequence
  Description of Artificial Sequence Synthetic peptide
QRETLKR

SEQ ID NO: 754 - protein, 7 aa, Artificial Sequence
  Description of Artificial Sequence Synthetic peptide
QAETLKR

SEQ ID NO: 755 - protein, 7 aa, Artificial Sequence
  Description of Artificial Sequence Synthetic peptide
QSAHLKR

SEQ ID NO: 756 - protein, 7 aa, Artificial Sequence
  Description of Artificial Sequence Synthetic peptide
LRDSLKR

SEQ ID NO: 757 - protein, 7 aa, Artificial Sequence
  Description of Artificial Sequence Synthetic peptide
VRHNLTR

SEQ ID NO: 758 - protein, 7 aa, Artificial Sequence
  Description of Artificial Sequence Synthetic peptide
VRHNLTR

SEQ ID NO: 759 - protein, 7 aa, Artificial Sequence
  Description of Artificial Sequence Synthetic peptide
QQTNLTR

SEQ ID NO: 760 - protein, 7 aa, Artificial Sequence
  Description of Artificial Sequence Synthetic peptide
LSQTLKR

SEQ ID NO: 761 - protein, 7 aa, Artificial Sequence
  Description of Artificial Sequence Synthetic peptide
QRSDLTR

SEQ ID NO: 762 - protein, 7 aa, Artificial Sequence
  Description of Artificial Sequence Synthetic peptide
QRPHLTN

SEQ ID NO: 763 - protein, 7 aa, Artificial Sequence
  Description of Artificial Sequence Synthetic peptide
QQTNLAR

SEQ ID NO: 764 - protein, 7 aa, Artificial Sequence
  Description of Artificial Sequence Synthetic peptide
DPSVLTR

SEQ ID NO: 765 - protein, 7 aa, Artificial Sequence
  Description of Artificial Sequence Synthetic peptide
DRSVLKR

SEQ ID NO: 766 - protein, 7 aa, Artificial Sequence
  Description of Artificial Sequence Synthetic peptide
ISHNLAR

SEQ ID NO: 767 - protein, 7 aa, Artificial Sequence
  Description of Artificial Sequence Synthetic peptide
VGSNLTR

SEQ ID NO: 768 - protein, 7 aa, Artificial Sequence
  Description of Artificial Sequence Synthetic peptide
ISHNLAR

SEQ ID NO: 769 - protein, 7 aa, Artificial Sequence
  Description of Artificial Sequence Synthetic peptide
VGSNLTR

SEQ ID NO: 770 - protein, 7 aa, Artificial Sequence
  Description of Artificial Sequence Synthetic peptide
QRNNLGR

SEQ ID NO: 771 - protein, 7 aa, Artificial Sequence
  Description of Artificial Sequence Synthetic peptide
DPSNLRR

SEQ ID NO: 772 - protein, 7 aa, Artificial Sequence
  Description of Artificial Sequence Synthetic peptide
QHPNLTR

SEQ ID NO: 773 - protein, 7 aa, Artificial Sequence
  Description of Artificial Sequence Synthetic peptide
QRNALAG

SEQ ID NO: 774 - protein, 7 aa, Artificial Sequence
  Description of Artificial Sequence Synthetic peptide
QRSDLHR

SEQ ID NO: 775 - protein, 7 aa, Artificial Sequence
  Description of Artificial Sequence Synthetic peptide
QRNALAG

SEQ ID NO: 776 - protein, 7 aa, Artificial Sequence
  Description of Artificial Sequence Synthetic peptide
RTDLLGR

SEQ ID NO: 777 - protein, 7 aa, Artificial Sequence
  Description of Artificial Sequence Synthetic peptide
RREGLGR

SEQ ID NO: 778 - protein, 7 aa, Artificial Sequence
  Description of Artificial Sequence Synthetic peptide
DRSSLRR

SEQ ID NO: 779 - protein, 7 aa, Artificial Sequence
  Description of Artificial Sequence Synthetic peptide
QRNTLKG

SEQ ID NO: 780 - protein, 7 aa, Artificial Sequence
  Description of Artificial Sequence Synthetic peptide
DPSNLRR

SEQ ID NO: 781 - protein, 7 aa, Artificial Sequence
  Description of Artificial Sequence Synthetic peptide
QGGTLVR

SEQ ID NO: 782 - protein, 7 aa, Artificial Sequence
  Description of Artificial Sequence Synthetic peptide
VGASLKR

SEQ ID NO: 783 - protein, 7 aa, Artificial Sequence
  Description of Artificial Sequence Synthetic peptide
LGENLRR

SEQ ID NO: 784 - protein, 7 aa, Artificial Sequence
  Description of Artificial Sequence Synthetic peptide
VGASLKR

SEQ ID NO: 785 - protein, 7 aa, Artificial Sequence
  Description of Artificial Sequence Synthetic peptide
VSNSLAR

SEQ ID NO: 786 - protein, 7 aa, Artificial Sequence
  Description of Artificial Sequence Synthetic peptide
DPTSLNR

SEQ ID NO: 787 - protein, 7 aa, Artificial Sequence
  Description of Artificial Sequence Synthetic peptide
QGNTLTR

SEQ ID NO: 788 - protein, 7 aa, Artificial Sequence
  Description of Artificial Sequence Synthetic peptide
LKHDLGR

SEQ ID NO: 789 - protein, 7 aa, Artificial Sequence
  Description of Artificial Sequence Synthetic peptide
DPSNLRR

SEQ ID NO: 790 - protein, 7 aa, Artificial Sequence
  Description of Artificial Sequence Synthetic peptide
VGASLKR

SEQ ID NO: 791 - protein, 7 aa, Artificial Sequence
  Description of Artificial Sequence Synthetic peptide
VGSNLTR

SEQ ID NO: 792 - protein, 7 aa, Artificial Sequence
  Description of Artificial Sequence Synthetic peptide
DPSNLRR

SEQ ID NO: 793 - protein, 7 aa, Artificial Sequence
  Description of Artificial Sequence Synthetic peptide
EGGNLMR

SEQ ID NO: 794 - DNA, 23 bp, Danio rerio
gccttctccggggcgcagaaggt

SEQ ID NO: 795 - DNA, 24 bp, Danio rerio
atcttctgctccaggggtgaaggt

SEQ ID NO: 796 - DNA, 23 bp, Danio rerio
agccgcagctctcggctgtagac

SEQ ID NO: 797 - DNA, 24 bp, Danio rerio
ctcttctcccccagagctgtggag

SEQ ID NO: 798 - DNA, 23 bp, Danio rerio
cccctcagcccagatgggaggag

SEQ ID NO: 799 - DNA, 23 bp, Danio rerio
cacacctgcacacagatgctgct

SEQ ID NO: 800 - DNA, 23 bp, Danio rerio
ggccacctccaccagcagcgggc

SEQ ID NO: 801 - DNA, 23 bp, Danio rerio
cccctctcctcaaagcagatgca

SEQ ID NO: 802 - DNA, 24 bp, Danio rerio
ggccccaccaagcctgctggagga

SEQ ID NO: 803 - DNA, 24 bp, Danio rerio
ctctgccgccacctagaggatggt

SEQ ID NO: 804 - DNA, 24 bp, Danio rerio
gcccacagcaatggcggagccgcc

SEQ ID NO: 805 - DNA, 23 bp, Danio rerio
gacttcctctctgtgcagtcggc

SEQ ID NO: 806 - DNA, 23 bp, Danio rerio
acctcctgcagtgtgaggttgtc

SEQ ID NO: 807 - DNA, 24 bp, Danio rerio
ggcgtccacgtacgagcggaggag

SEQ ID NO: 808 - DNA, 23 bp, Danio rerio
ttcctcctcctgatgcggaggct

SEQ ID NO: 809 - DNA, 23 bp, Danio rerio
ttctgcagctcaatggagatggt

SEQ ID NO: 810 - DNA, 23 bp, Danio rerio
agcttcctccgccggaagttgag

SEQ ID NO: 811 - DNA, 24 bp, Danio rerio
gtcctcctcatggcggtcgatggt

SEQ ID NO: 812 - DNA, 23 bp, Danio rerio
tcccactgctgattgtaggtgga

SEQ ID NO: 813 - DNA, 23 bp, Danio rerio
aaccgcaccacacagtggaagag

SEQ ID NO: 814 - DNA, 24 bp, Danio rerio
aactacaacattagggctggagga

SEQ ID NO: 815 - DNA, 23 bp, Danio rerio
gtcctccccttcaagtcgaatag

SEQ ID NO: 816 - DNA, 24 bp, Danio rerio
ctcctcctcaaacacgaagctgtc

SEQ ID NO: 817 - DNA, 23 bp, Danio rerio
agcagctgcatgggggggatgaa

SEQ ID NO: 818 - DNA, 23 bp, Glycine max
tgcttcatcacaatggagatgat

SEQ ID NO: 819 - DNA, 23 bp, Glycine max
tgcttcatcacaatggagatgat

SEQ ID NO: 820 - DNA, 23 bp, Arabidopsis thaliana
ctccacaacatcaggatgacgaa

SEQ ID NO: 821 - DNA, 24 bp, Arabidopsis thaliana
ctcttcgtcctatcggcagaggcg

SEQ ID NO: 822 - DNA, 24 bp, Arabidopsis thaliana
gccagcgacggtggtggtggtggc

SEQ ID NO: 823 - DNA, 23 bp, Arabidopsis thaliana
ttcttcatccagatgttgttgag

SEQ ID NO: 824 - DNA, 24 bp, Arabidopsis thaliana
cccttccacaacaacggagaagct

SEQ ID NO: 825 - DNA, 24 bp, Arabidopsis thaliana
agctacgccgtagccggagacgcc

SEQ ID NO: 826 - DNA, 24 bp, Arabidopsis thaliana
ggcacctccgatttcgtggaggaa

SEQ ID NO: 827 - DNA, 23 bp, Arabidopsis thaliana
tcctccaccgaatcgacggcgct

SEQ ID NO: 828 - DNA, 24 bp, Arabidopsis thaliana
ttcctccaccgaatcgacggcgct

SEQ ID NO: 829 - DNA, 23 bp, Arabidopsis thaliana
gtctccgcctaggagatgcagac

SEQ ID NO: 830 - DNA, 23 bp, Arabidopsis thaliana
tgcttcttcatccagatgttgtt

SEQ ID NO: 831 - DNA, 24 bp, Arabidopsis thaliana
ctcttcgtcctatcggtagaggcg

SEQ ID NO: 832 - DNA, 23 bp, Arabidopsis thaliana
ttcgtcttcgagtcgtcgttgtt

SEQ ID NO: 833 - DNA, 21 bp, Artificial Sequence
  Description of Artificial Sequence Synthetic oligonucleotide
gagcgccccttccagtgtcgc

SEQ ID NO: 834 - DNA, 22 bp, Artificial Sequence
  Description of Artificial Sequence Synthetic oligonucleotide
tcggcattggaatggcttctcg

SEQ ID NO: 835 - DNA, 23 bp, Artificial Sequence
  Description of Artificial Sequence Synthetic oligonucleotide
gccattccaatgccgaatatgca

SEQ ID NO: 836 - DNA, 22 bp, Artificial Sequence
  Description of Artificial Sequence Synthetic oligonucleotide
ccctcaggtgggtttttaggtg

SEQ ID NO: 837 - DNA, 24 bp, Artificial Sequence
  Description of Artificial Sequence Synthetic oligonucleotide
ggggagcgccccttccagtgtcgc

SEQ ID NO: 838 - DNA, 34 bp, Artificial Sequence
  Description of Artificial Sequence Synthetic oligonucleotide
gtgcagaggatcccctcaggtgggtttttaggtg

SEQ ID NO: 839 - DNA, 25 bp, Artificial Sequence
  Description of Artificial Sequence Synthetic oligonucleotide
gggtagtacgatgacggaacctgtc

SEQ ID NO: 840 - protein, 25 aa, Artificial Sequence
  Description of Artificial Sequence Synthetic peptide
CXXXXCXXXXXXXXXXXXHXXXXXH

SEQ ID NO: 841 - protein, 11 aa, Artificial Sequence
  Description of Artificial Sequence Synthetic peptide
FQCRICMRNFS

SEQ ID NO: 842 - protein, 5 aa, Artificial Sequence
  Description of Artificial Sequence Synthetic peptide
HTRTH

SEQ ID NO: 843 - protein, 5 aa, Artificial Sequence
  Description of Artificial Sequence Synthetic peptide
HLRTH

SEQ ID NO: 844 - protein, 5 aa, Artificial Sequence
  Description of Artificial Sequence Synthetic peptide
HLKTH

(19) United States
(12) Patent Application Publication
(10) Pub. No.: US 2012/0178647 A1
(43) Pub. Date: Jul. 12, 2012

(54) ENGINEERING OF ZINC FINGER ARRAYS BY CONTEXT-DEPENDENT ASSEMBLY

(75) Inventors: J. Keith Joung, Winchester, MA (US); Jeffry D. Sander, Woburn, MA (US)

(73) Assignee: THE GENERAL HOSPITAL CORPORATION, Boston, MA (US)

(21) Appl. No.: 13/386,995

(22) PCT Filed: Aug. 3, 2010
(86) PCT No.: PCT/US2010/044205

§ 371 Date: Mar. 26, 2012

Related U.S. Application Data
(60) Provisional application No. 61/230,887, filed on Aug. 3, 2009.

(51) Int. Cl. C40B 30/04; C12N 15/62; C07K 19/00; C12N 9/10; C12N 5/071; C12N 9/16; C12N 9/14; C12P 19/34; C40B 40/10; C12P 21/00; C12N 9/12

(52) U.S. Cl. 506/9; 435/69.7; 536/25.3; 530/350; 435/193; 435/194; 435/196; 435/195; 435/91.53; 506/18; 435/375

(57) ABSTRACT

A method of designing a multi-zinc-finger polypeptide predicted to bind to a sequence of interest that has at least three subsites includes the steps of: a) providing a nucleotide sequence of interest having first, second, and third consecutive subsites, wherein each of the first and third subsites are adjacent to the second subsite; b) identifying first and second adjacent zinc finger polypeptide sequences previously shown to bind to the first and second subsites in the context of a multi-zinc finger polypeptide; c) identifying a third zinc finger polypeptide previously shown to bind to a third subsite adjacent to the second subsite when present in the context of a multi-zinc finger polypeptide adjacent to the second zinc finger polypeptide; and d) combining the first, second, and third zinc finger polypeptide sequences in linear order, thereby designing a multi-zinc finger polypeptide predicted to bind to the sequence of interest.

### CLAIM OF PRIORITY

This application claims priority to U.S. Patent Application Ser. No. 61/230,887, filed on Aug. 3, 2009, the entire contents of which are hereby incorporated by reference.

### STATEMENT AS TO FEDERALLY SPONSORED RESEARCH

This invention was made with government support under grant numbers C009216, GM069906, GM088040, and GM078369 awarded by the National Institutes of Health. The government has certain rights in the invention.

### TECHNICAL FIELD

This invention relates to methods of engineering DNA-binding proteins that include zinc finger arrays.

### BACKGROUND

Zinc finger proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci.

USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

Widespread adoption and large-scale use of zinc finger protein technology have been hindered by the continued lack of a robust, easy-to-use, and publicly available method for engineering zinc finger arrays. One existing approach, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660), but the building and screening of such combinatorial libraries requires significantly greater labor and expertise than modular assembly approaches. There is a need for a robust, easy-to-use method for engineering zinc finger arrays.

### SUMMARY

This disclosure describes a new platform for context-dependent design of zinc finger proteins that is as simple to practice as modular assembly but that possesses a high success rate comparable to combinatorial selection-based methods. In the methods described herein, multi-finger arrays are assembled together by using an archive of zinc finger units that have been pre-determined to work well with one another. In contrast to modular assembly, the disclosed context-dependent assembly (CoDA) methods do not treat fingers as independent modules. Instead, the choice of finger units used to assemble an array is explicitly determined by the identity of neighboring fingers, a strategy that strictly accounts for potential context-dependent effects between neighboring fingers and thereby increases the probability that the multi-finger array will function well when assembled together. The disclosed methods are rapid and require no specialized expertise.

In one aspect, the invention features methods of designing a multi-zinc-finger polypeptide sequence predicted to bind to a nucleic acid sequence of interest that includes at least three subsites. The methods include the steps of a) providing a nucleotide sequence of interest having first, second, and third consecutive subsites, wherein each of the first and third subsites are adjacent to the second subsite; b) identifying first and second adjacent zinc finger polypeptide sequences previously shown to bind to the first and second subsites in the context of a multi-zinc finger polypeptide; c) identifying a third zinc finger polypeptide sequence shown to bind to a third subsite adjacent to the second subsite when present in the context of a multi-zinc finger polypeptide adjacent to the second zinc finger polypeptide sequence; and d) combining the first, second, and third zinc finger polypeptide sequences in linear order, thereby designing a multi-zinc finger polypeptide sequence predicted to bind to the sequence of interest. In some embodiments, the first subsite is located 5′ to the second subsite and/or the first zinc finger polypeptide sequence is located amino-terminal to the second zinc finger polypeptide sequence. In some embodiments, the first subsite is located 3′ to the second subsite and/or the first zinc finger polypeptide sequence is located carboxy-terminal to the second zinc finger polypeptide sequence.

In some embodiments, the second zinc finger sequence includes a sequence selected from SEQ ID NOs: 1-18. In some embodiments, the first zinc finger sequence includes a sequence selected from SEQ ID NOs: 19-337. In some embodiments, the third zinc finger sequence comprises a sequence selected from SEQ ID NOs: 338-681.

In some embodiments, the methods further include causing to be produced or producing a polynucleotide comprising a sequence that encodes a polypeptide comprising the multi-zinc-finger polypeptide, causing to be produced or producing a polypeptide comprising the multi-zinc-finger polypeptide sequence, and polypeptides and polynucleotides designed and/or produced by the methods described herein. The polypeptides include one or more functional domains (e.g., a transcriptional activation domain, endonuclease domain, transcriptional repressor domain, transcriptional silencing domain, acetylase domain, de-acetylase domain, methylation domain, de-methylation domain, kinase domain, phosphatase domain, dimerization domain, multimerization domain, nuclear localization domain, nuclease domain, endonuclease domain, resolvase domain, or integrase domain).

In another aspect, the invention features polypeptides having two or more zinc finger domains, wherein one of the zinc finger domains includes a recognition helix sequence selected from SEQ ID NOs: 1-18, and one or more associated recognition helix sequence selected from SEQ ID NOs: 19-337 and/or SEQ ID NOs: 338-681. In some embodiments, the zinc finger domains are associated as shown in FIGS. 3 and 4. In some embodiments, a sequence selected from SEQ ID NOs: 19-337 is located amino-terminal to a sequence selected from SEQ ID NOs: 1-18. In some embodiments, a sequence selected from SEQ ID NOs: 338-681 is located carboxy-terminal to a sequence selected from SEQ ID NOs: 1-18. In some embodiments, a sequence selected from SEQ ID NOs: 19-337 is located amino-terminal to a sequence selected from SEQ ID NOs: 1-18, and a sequence selected from SEQ ID NOs: 338-681 is located carboxy-terminal to the sequence selected from SEQ ID NOs: 1-18. In some embodiments, one or more of the zinc finger domains include the motif Cys-(X)₂₋₄-Cys-(X)₁₂-His-(X)₃₋₅-His (SEQ ID NO:840). In some embodiments, the zinc finger domains include one or more sequences selected form SEQ ID NOs: 841-844. In some embodiments, the polypeptides include one or more functional domains (e.g., a transcriptional activation domain, endonuclease domain, transcriptional repressor domain, transcriptional silencing domain, acetylase domain, de-acetylase domain, methylation domain, de-methylation domain, kinase domain, phosphatase domain, dimerization domain, multimerization domain, nuclear localization domain, nuclease domain, endonuclease domain, resolvase domain, or integrase domain).

In a further aspect, the invention features methods of regulating the expression of a gene that include contacting a polypeptide as described herein with a sequence of interest within the gene to form a binding complex, such that expression of the gene is regulated.

In another aspect, the invention features methods of altering the structure of a gene that include contacting a zinc finger polypeptide as described herein with a sequence of interest in the gene to form a binding complex, such that the structure of the gene is altered.

In a further aspect, the invention features methods of cleaving a sequence of interest that include contacting a zinc finger polypeptide as described herein with the sequence of interest to form a binding complex, such that the sequence of interest is cleaved. The methods can be used to create mutations (e.g., insertion or deletion mutations) in the sequence of interest.

In another aspect, the invention features a polypeptide or polynucleotide as described herein for use in therapy, e.g., for use in therapy of a disorder as described herein.

In another aspect, the invention features a set, archive, or library of multi-zinc finger array sequences, wherein each array comprises at least first, second, and third adjacent zinc fingers, wherein the sequence of the second zinc finger is identical for each entry in the database, and wherein the database comprises at least three (e.g., at least five, ten, fifteen, twenty, 25, 40, 60, 80, 100, 150, 200, 500, or 1000) entries. In some embodiments, the set, archive, or library comprises sequences as shown in FIGS. 3A-4B.

In another aspect, the invention features a set, archive, or library of adjacent zinc finger sequence modules, wherein each module comprises two adjacent zinc fingers, wherein the sequence of the first or second zinc finger is identical for each entry in the database, and wherein the database comprises at least three (e.g., at least five, ten, fifteen, twenty, 25, 40, 60, 80, 100, 150, 200, 500, or 1000) entries. In some embodiments, the set, archive, or library comprises sequences as shown in FIGS. 3A and 3B or FIGS. 4A and 4B.

In a further aspect, the invention features a methods of creating a set of multi-zinc-finger array sequences. The methods include providing a parent zinc finger polypeptide having at least first, second, and third adjacent zinc fingers, wherein the zinc finger polypeptide binds to a known parental target sequence comprising at least first, second, and third adjacent subsites; producing a library of zinc finger polypeptides based on the parent zinc finger polypeptide sequence, wherein each member of the library comprises the parental second zinc finger sequence and the sequence of either or both of the first and third fingers are varied; and selecting members of the library of zinc finger polypeptides that bind to one or more target sequences comprising the parental second subsite and either or both of a non-parental first and third subsite, thereby providing a set of multi-zinc-finger array sequences with common second finger sequences. In some embodiments, the library is expressed in vitro. In some embodiments, the library is expressed in an expression system selected from the group consisting of eukaryotic, prokaryotic and viral expression systems. In some embodiments, the library is expressed in bacteria (e.g., E. coli).

The term “zinc finger” or “Zf” refers to a polypeptide comprising a DNA binding domain that is stabilized by zinc. The individual DNA binding domains are typically referred to as “fingers.” A Zf protein has at least one finger, preferably two fingers, three fingers, or six fingers. A Zf protein having two or more Zfs is referred to as a “multi-finger” or “multi-Zf” protein or a “zinc finger array.” Each finger typically comprises an approximately 30 amino acid, zinc-chelating, DNA-binding domain. An exemplary motif characterizing one class of these proteins is -Cys-(X)₂₋₄-Cys-(X)₁₂-His-(X)₃₋₅-His (SEQ ID NO:840), where X is any amino acid, which is known as the “C(2)H(2)” class. Studies have demonstrated that a single Zf of this class consists of an alpha helix containing the two invariant histidine residues co-ordinated with zinc along with the two cysteine residues of a single beta turn (see, e.g., Berg and Shi, 1996, Science 271:1081-85). The portion of the alpha helix that can make sequence specific contacts to DNA bases is called the “recognition helix.” “Adjacent” zinc fingers are those that are present sequentially in a zinc finger polypeptide array without an intervening zinc finger polypeptide sequence. For example, in a three-zinc-finger array, fingers 1 and 2 are adjacent and fingers 2 and 3 are also adjacent.

Each finger within a Zf protein binds to from about two to about five base pairs within a DNA sequence. Typically a single Zf within a Zf protein binds to a three or four base pair “subsite” within a DNA sequence. Accordingly, a “subsite” is a DNA sequence that is bound by a single zinc finger. A “multi-subsite” is a DNA sequence that is bound by more than one zinc finger, and comprises at least 4 bp, preferably 6 bp or more. A multi-Zf protein binds at least two, and typically three, four, five, six or more subsites i.e., one for each finger of the protein. “Adjacent” subsites are those that are bound by adjacent zinc fingers.

The present invention provides methods for the engineering of zinc finger proteins that bind to a desired nucleotide sequence comprising several subsites, which is referred to herein as a “sequence of interest.” A “sequence of interest” may be located within a “gene of interest.” For example, in one embodiment a “sequence of interest” is a string of consecutive subsites located in the vicinity of the promoter of a gene of interest. In another embodiment, a sequence of interest may be located within the coding region of a gene of interest. However, the “sequence of interest” need not be located in a natural gene, but can be any sequence chosen as the binding site of an engineered zinc finger protein, using the methods of the present invention. For example, in one embodiment, the methods of the present invention can be used to select a Zf protein that binds to a specific sequence in a piece of DNA that has been artificially altered, such as a recombinant DNA molecule in a vector, or a manipulated nucleotide sequence in a transgenic animal.

As used herein the term “target site” refers to any nucleic acid sequence bound by a Zf protein, and encompasses “sequences of interest.” For example, target sites may be artificially created nucleotide sequences that are used solely at certain stages in the selection procedure, and are not the actual “sequence of interest” to which the final selected Zf protein will bind.

The term “recombinant” when used herein with reference to portions of a nucleic acid or protein, indicates that the nucleic acid comprises two or more sub-sequences that are not found in the same relationship to each other in nature. For instance, a nucleic acid that is recombinantly produced typically has two or more sequences from distinct genes or non-adjacent regions of the same gene, synthetically arranged to make a new nucleic acid sequence encoding a new protein, for example, a DBD from one source and a regulatory or functional region from another source, or a Zf from the native Zif268 protein and a Zf selected from a library. The term “recombination” as used herein, refers to the process of producing a recombinant protein or nucleic acid by standard techniques known to those skilled in the art, and described in, for example, Sambrook et al., Molecular Cloning; A Laboratory Manual 3d ed. (2001). The term “chimeric” as used herein refers to a protein containing at least two component portions or domains which are mutually heterologous in the sense that they do not occur together in precisely the same arrangement in nature. More specifically, the component portions are not found in the same continuous polypeptide sequence or molecule in nature, at least not in the same order or orientation or with the same spacing present in the chimeric protein. Typically, the chimeric proteins of the present invention contain a Zf DNA binding domain and at least one additional domain.

“K_{D}” refers to the dissociation constant for binding of one molecule to another molecule, i.e., the concentration of a molecule (such as a Zf protein), that gives half maximal binding to its binding partner (such as a DNA target sequence) under a given set of conditions. The K_{D} provides a measure of the strength of the interaction between two molecules, or the “affinity” of the interaction between two molecules. Two molecules that bind strongly to each other have a “high affinity” for each other, while molecules that bind weakly to each other have a “low affinity” for each other.

“Specific” or “specific-binding” as used herein, refers to the interaction between a protein and a nucleic acid wherein the protein recognizes and interacts with a defined nucleotide sequence, as opposed to a “non-specific” interaction wherein the protein does not require a defined nucleotide sequence to associate with the nucleic acid molecule (for example, in the extreme, a protein that interacts with the phosphate-sugar backbone of the DNA but not the bases of the nucleotides). The strength of the association between the protein and the nucleic acid molecule can vary significantly between different “binding complexes.” A “binding complex,” as used herein, comprises an association between a sequence of interest, target site or subsite and a Zf binding domain. “Binding complexes” can comprise both weakly-bound Zf proteins and nucleic acids and strongly-bound Zf proteins and nucleic acids. The strength or “affinity” of the association of a Zf with an intended or specified sequence of interest, target site or subsite is expressed in terms of the K_{D}, as defined above.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic diagram depicting assembly of a zinc finger array by combining amino-terminal (F1) and carboxy-terminal (F3) fingers that have each been previously identified in other three-finger arrays containing a common middle (F2) finger.

FIG. 2 is a schematic diagram depicting a database of pre-selected multi finger arrays with constant F2 position fingers and a method of assembling finger arrays for novel target sites.

FIGS. 3A-B are a table of recognition helix sequences of F1 units selected to bind specific three by subsites (top row), each identified from an active three-finger array in which it was positioned adjacent to the F2 unit shown in the grey column on the far left. N.F. indicates where selections were attempted to isolate a unit but “no finger” was obtained. “−” indicates that no attempt has yet been made to identify fingers. The sequences are associated with sequence identifiers by column as follows: F2, SEQ ID NOs: 1-18; GGG, SEQ ID NOs: 19-34; GGA, SEQ ID NOs: 35-50; GGC, SEQ ID NOs: 51-67; GGT, SEQ ID NOs: 68-82; GAG, SEQ ID NOs: 83-97; GAA, SEQ ID NOs: 98-114; GAC, SEQ ID NOs: 115-130; GAT, SEQ ID NOs: 131-136; GCG, SEQ ID NOs: 137-152; GCA, SEQ ID NOs: 153-167; GCC, SEQ ID NOs: 177-183; GCT, SEQ ID NOs: 184-200; GTG, SEQ ID NOs: 201-217; GTA, SEQ ID NOs: 218-232; GTC, SEQ ID NOs: 233-248; GTT, SEQ ID NOs: 249-263; AGG, SEQ ID NOs: 264-273; AAC, SEQ ID NOs: 274-285; ACG, SEQ ID NOs: 286-297; TGC, SEQ ID NOs: 298-309; TGT, SEQ ID NOs: 310-323; TAG, SEQ ID NOs: 324-332; TCG, SEQ ID NOs: 333-335; TCT, SEQ ID NO:336; TTC, SEQ ID NO:337.

FIGS. 4A-B are a table of recognition helix sequences of F3 units selected to bind specific three by subsites (top row), each identified from an active three-finger array in which it was positioned adjacent to the F2 unit shown in the grey column on the far left. N.F. indicates where selections were attempted to isolate a unit but “no finger” was obtained. “−” indicates that no attempt has yet been made to identify fingers. The sequences are associated with sequence identifiers by column as follows: F2, SEQ ID NOs: 1-18; GGG, SEQ ID NOs: 338-353; GGA, SEQ ID NOs: 354-368; GGC, SEQ ID NOs: 369-382; GGT, SEQ ID NOs: 383-398; GAG, SEQ ID NOs: 399-416; GAA, SEQ ID NOs: 417-432; GAC, SEQ ID NOs: 433-449; GAT, SEQ ID NOs: 450-464; GCG, SEQ ID NOs: 465-480; GCA, SEQ ID NOs: 481-496; GCC, SEQ ID NOs: 497-512; GCT, SEQ ID NOs: 513-527; GTG, SEQ ID NOs: 528-543; GTA, SEQ ID NOs: 544-559; GTC, SEQ ID NOs: 560-575; GTT, SEQ ID NOs: 576-590; TGG, SEQ ID NOs: 591-606; TGC, SEQ ID NOs: 607-617; TGT, SEQ ID NOs: 618-633; TAG, SEQ ID NOs: 634-646; TAA, SEQ ID NOs: 647-659; TCG, SEQ ID NOs: 660-675; TCC, SEQ ID NO:676; TCT, SEQ ID NOs: 677-678; TTA, SEQ ID NO:679; TTC, SEQ ID NO:680; TTT, SEQ ID NO:681.

FIG. 5 is a table depicting engineered zinc finger arrays with their respective target binding sites, F1, F2, and F3 recognition helix (RH) sequences, and whether or not the arrays were active in a bacterial two-hybrid (B2H) assay. The sequences are associated with sequence identifiers by column as follows: Target sites, SEQ ID NOs: 682-709; F1 RH sequences, SEQ ID NOs: 710-737; F2 RH sequences, SEQ ID NOs: 738-765; F3 RH sequences, SEQ ID NOs: 766-793.

FIG. 6 is a bar graph depicting fold-activation of a lacZ reporter gene in the B2H system of 181 zinc finger arrays constructed by CoDA. Values of B2H activity are plotted from lowest to highest from left to right. Thresholds of fold-activation that predict failure (<1.57) or success (>3.00) as ZFNs are shown in red and green, respectively. Target sites bound by the 181 zinc finger arrays tested are shown in FIGS. 7A-B.

FIGS. 7A-B are a table depicting target sites and bacterial two-hybrid (B2H) reporter assay activities (reported as fold-activation of a lacZ reporter gene) for 181 CoDA zinc finger arrays engineered using CoDA. For most of the zinc finger arrays, IPTG was added to the culture medium at 500 μM to induce zinc finger protein expression in the B2H reporter assay as previously described. For some arrays, a lower concentration of IPTG was used as indicated to minimize toxicity associated with zinc finger array expression.

FIG. 8 is a table depicting a comparison of modularly assembled and CoDA zinc finger arrays in a bacterial two-hybrid (B2H) reporter assay. Fold-activation values for zinc finger arrays targeted to 26 different DNA sites (left column) are shown. Zinc finger arrays were made by either modular assembly (using one of three module archives from Sangamo, Barbas, or Toolgen) or CoDA. For each of the 26 target sites, the most active array (as judged by B2H fold-activation values) is shaded. The number of sites and the percentage of sites for which CoDA or a particular module set yielded the most active protein are shown in the third-to-last and second-to-last rows, respectively. The average fold-activation values for all arrays made by a CoDA or a particular modular set are shown in the last row of the table.

FIGS. 9A and B are bar graphs depicting fold-activation values (as measured in the B2H reporter assay) of the most active modularly assembled zinc finger arrays (9A) and activation values of CoDA arrays (9B) for each of the 26 target sites (listed in FIG. 8). In the right panel, fold-activation values of CoDA arrays for the same 26 target sites are shown. The fold-activation values are arranged from lowest to highest going from left to right. Thresholds of fold-activation that predict failure (<1.57) or success (>3.00) as ZFNs are shown in red and green, respectively.

FIG. 10 is a table depicting endogenous zebrafish genes targeted by CoDA ZFNs. Target sites within each gene are written 5′ to 3′ with the two half-sites targeted by the zinc finger arrays shown in upper case letters and the intervening spacer sequence shown in lower case. The target site sequences are associated with SEQ ID NOs: 794-817, respectively.

FIG. 11 is a table depicting endogenous plant (soybean and Arabidopsis) genes targeted by CoDA ZFNs. Target sites within each gene are written 5′ to 3′ with the two half-sites targeted by the zinc finger arrays shown in upper case letters and the intervening spacer sequence shown in lower case. The target site sequences are associated with SEQ ID NOs: 818-832, respectively.

### DETAILED DESCRIPTION

Described herein is a new platform for context-dependent design of zinc finger proteins that is as simple to practice as modular assembly but that possesses a high success rate comparable to selection-based methods. In the methods described herein, multi-finger arrays are assembled together by using an archive zinc finger units that have been pre-determined to work well with one another, thereby explicitly accounting for the context-dependent activities of zinc fingers in a multi-finger array.

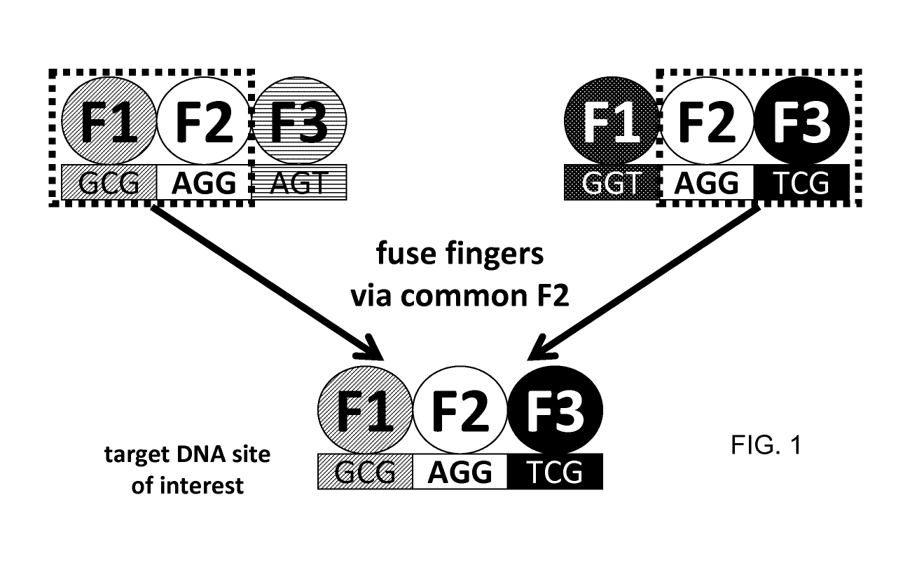

The fundamental strategy underlying the new methods is to assemble amino-terminal (F1) and carboxy-terminal (F3) fingers that have each been previously identified in other three-finger arrays containing a common middle (F2) finger. For example, FIG. 1 shows two different three-finger arrays, each identified as binding different 9 base pair target sites and that each share a common middle F2 and associated subsite. A three-finger array with a new sequence specificity can be made by joining together the amino-terminal finger (F1) from the first array, the middle finger common to both arrays (F2), and the carboxy-terminal finger (F3) from the second array (FIG. 1). In the resulting three-finger array, the F1 and F3 units have both been previously established to work well with the shared fixed F2, thereby accounting for context-dependence between adjacent fingers and increasing the probability that the assembled three fingers will work well together.

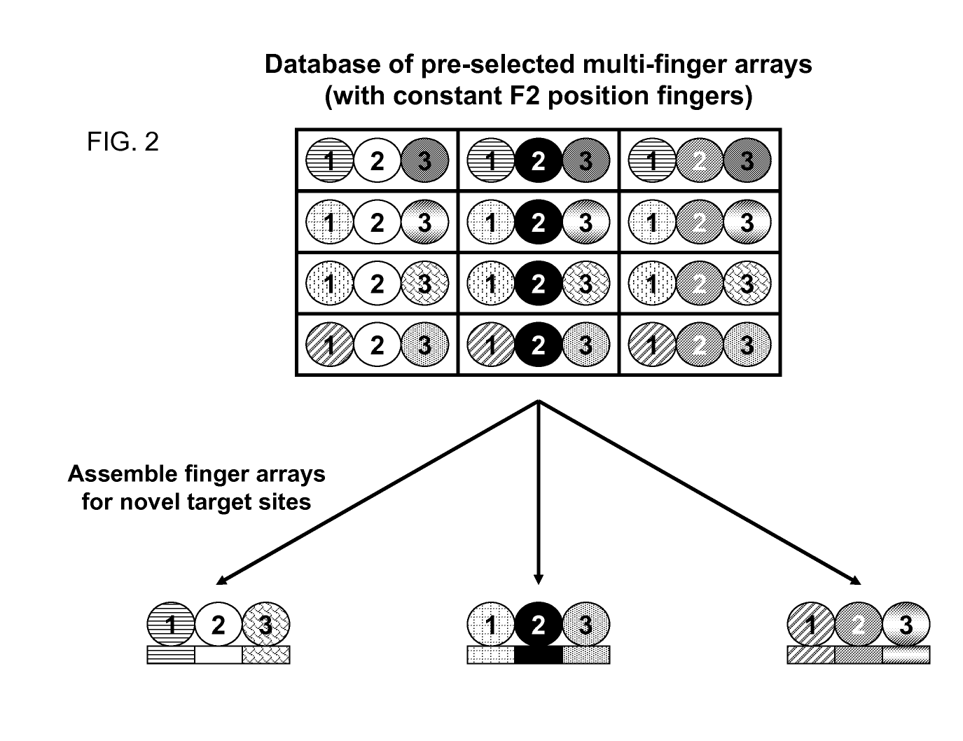

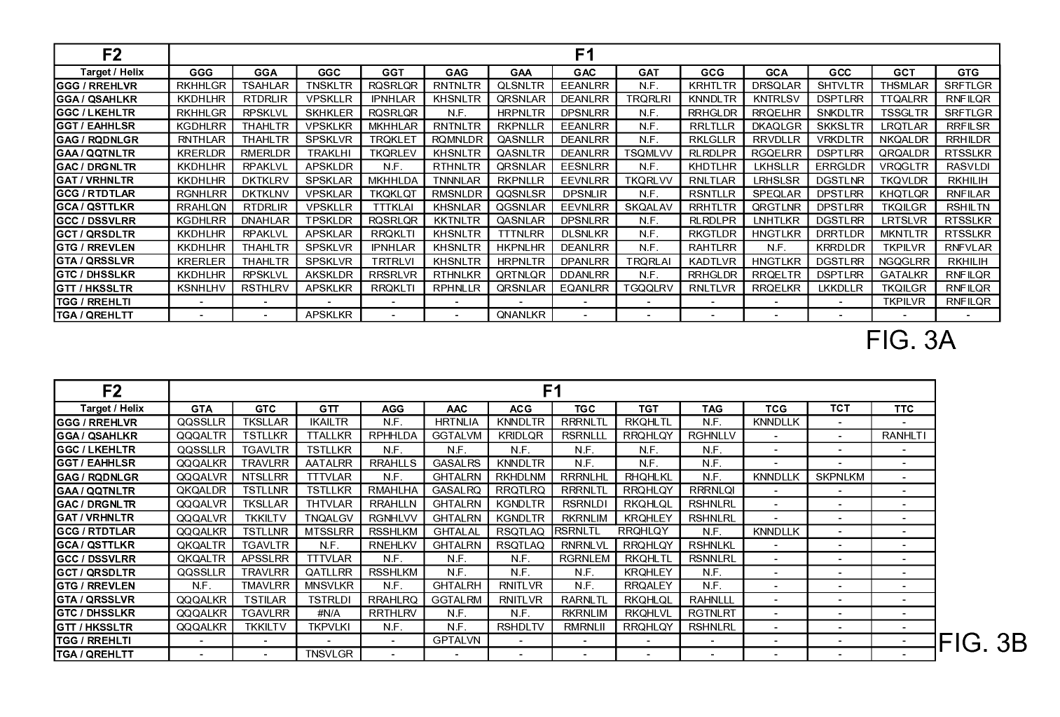

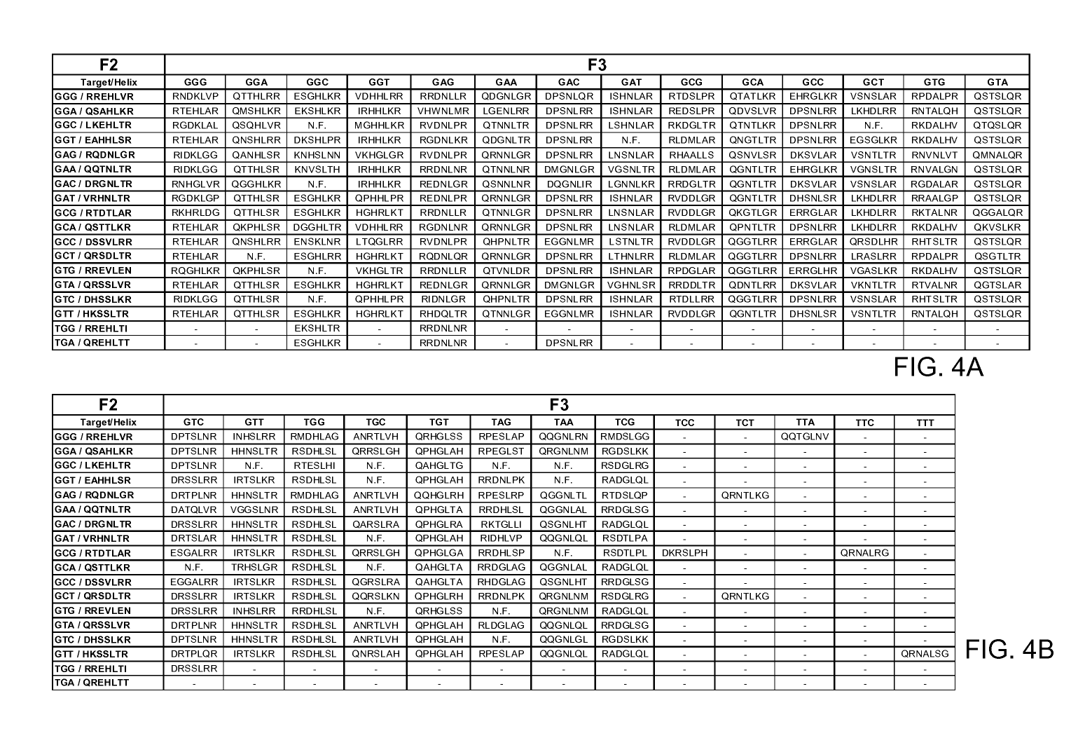

In some embodiments, a database of pre-selected multi-finger arrays with constant F2 position fingers can be used to engineer zinc finger arrays for novel target sites (see FIG. 2). The database can include several F2 fingers identified as recognizing different subsite sequences, along with F1 and F3 fingers and their associated subsites. To design a three-finger array, one simply selects an F2 finger specific for the middle subsite of the sequence of interest and F1 and F3 fingers that bound to the first and third subsites. Because the methods account for the context dependence of adjacent fingers, it is not necessary that a full three-finger protein that binds to the sequence of interest have been previously selected. An exemplary database is provided in FIGS. 3 and 4, which can be used to design zinc-finger arrays for a large number of sequences of interest.

Additionally, the methods described herein can be repeated to design zinc finger arrays with more than three fingers. For example, the methods can be used to design zinc finger arrays with four, five, six, seven, eight, nine, or more fingers. To design an array with more than three fingers, the method is repeated for each set of three adjacent fingers in the array. For example, when an array of four fingers is designed, the method can be performed by assembling the N-terminal three fingers with a common F2, then defining the third finger from the N-terminus as the new F2 for assembling the C-terminal three fingers. Alternatively, the C-terminal three fingers can be assembled first, followed by the N-terminal three fingers. For longer arrays, the sequences can be designed in any order, assembling in three-finger “windows” until the entire array is assembled. When an array that includes five fingers is designed, the method can be performed by assembling two three-finger units (F1-F2-F3 and F1′-F2′-F3′), wherein F3 and F1′ share the same sequence and target site specificity, to provide the five-finger array F1-F2-F3-F2′-F3′.

The methods described herein for assembling zinc finger arrays can be performed by hand or using the assistance of a computer program such as the Zinc Finger Targeter program (ZiFiT V3.3) (Sander et al., 2010, Nucleic Acids Res., doi:10.1093/nar/gkq319; Sander et al., 2007, Nucleic Acids Res. 35:W599-605). Such computer programs can be modified to incorporate the design parameters described herein. In some embodiments, the computer program can scan a larger nucleic acid sequence to provide the sequence of potential CoDA target sites and unique identification numbers for plasmids encoding the finger units that can be used to assemble the arrays. In some embodiments, the computer program can generate DNA sequences encoding zinc finger arrays designed by the methods described herein required to target a given site or sites. These DNA fragments can then be synthesized by a commercial provider and cloned into existing expression vectors, such as those disclosed in Wright et al., 2006, Nat. Protoc., 1:1637-1652; Maeder et al., 2008, Mol. Cell, 31:294-301; Maeder et al., 2009, Nat. Protoc., 4:1471-1501; and Foley et al., 2009, PLoS ONE, 4:e4348.

### Zinc Finger Archives

Any zinc finger proteins with known sequences and target binding sites can be used in the methods described herein as a member of an archive of zinc finger units to engineer zinc finger arrays with new specificities. The only requirement is that the sequences share a zinc finger (e.g., F2) with identical amino acid sequence.

In some embodiments, some of the members of an archive of zinc fingers are identified by a screening or selection method, e.g., as described in Rebar et al., 1994, Science, 263:671; Choo et al., 1994, Proc. Natl. Acad. Sci. USA, 91:11163; Jamieson et al., 1994, Biochemistry, 33:5689; Wu et al., 1995, Proc. Natl. Acad. Sci. USA, 92:344; Isalan et al., 2001, Nat. Biotechnol., 19: 656; Greisman et al., 1997, Science, 275:657; Joung et al., 2000, Proc. Natl. Acad. Sci. USA, 97: 7382-87; Hurt et al., 2003, Proc. Natl. Acad. Sci. USA, 100: 12271-76; Maeder et al., 2008, Mol. Cell, 31: 394-301; U.S. Pat. No. 6,410,248; and US 2007/0178454. Such screening methods typically utilize large Zf libraries in which the key amino acids required for DNA binding have been randomized. One method that can be used for selection is phage display technology, in which the proteins encoded by the Zf library are expressed on the surface of the bacteriophage. Phage particles displaying Zf motifs with the desired sequence specificity are identified using standard techniques that select on the basis of DNA binding affinity and specificity and are then subjected to multiple rounds of selection and amplification.

More recently a bacterial “two-hybrid” method has been developed for selecting zinc finger proteins. In this system Zf-DNA interactions are required for cell growth and survival (Joung et al., 2000, Proc. Natl. Acad. Sci. USA, 97:7382 and US 2002/0119498). The bacterial two-hybrid system has an extremely low background rate and, because it does not require multiple rounds of selection and amplification, it is significantly faster to perform than phage display methods. Furthermore, the bacterial two-hybrid system has an added advantage in that, unlike phage display, the Zf-DNA binding interaction occurs within living cells.

Selection or screening methods can be used to generate an archive of zinc finger proteins that can be used in the methods described herein. In such methods, one finger of a multi-finger (e.g., three-finger) array is held constant along with its cognate binding subsite. The fingers adjacent to the constant finger are randomized and selected for binding to new subsites adjacent to the constant subsite. In an exemplary embodiment, the multi-finger array is a three-finger array, the F2 finger is held constant, and the F1 and F3 fingers are selected for binding to new subsites. By this method, an archive of several zinc finger proteins with identical F2 fingers can be generated for use in the CoDA methods described herein.

### Choice of the “Sequence of Interest”

In a preferred embodiment, the sequence of interest is chosen from a genomic “address” or location that is within or proximal to, for example, a “gene of interest,” such that ideally the sequence is statistically unique enough to occur only once in the genome. This ability to specify a unique sequence is a function of the length of the target site and the size of the genome or other desired substrate (such as a nucleic acid vector, for example). For example, assuming random base distribution, a unique 16 by sequence will occur only once in 4.3×10⁹ bp, thus a 16 by sequence should be sufficient to specify a unique address within 4.3×10⁹ by of random sequence. Similarly, an 18 by address would enable sequence specific targeting within 6.8×10¹⁰ by of DNA. The unique sequence of interest selected can be located anywhere within or proximal to the gene of interest. Wherein the ultimate aim is to generate a synthetic transcription factor or nuclease to regulate expression or sequence, respectively, of the gene of interest, it is preferable that the chosen sequence of interest is within the general vicinity of the promoter and in a region where chromatin architecture will not impede binding of the Zf protein to the DNA (see for example, Liu et al., 2001, J. Biol. Chem., 276:11323). Where the aim is to design a zinc finger nuclease for creation of an insertion or deletion mutation in the gene of interest, the chosen sequence of interest can also be within a coding sequence of the gene of interest or a non-coding expression control region of the gene of interest.

A sequence of interest can be located in any gene or other nucleic acid sequence (such as a vector). For example, a sequence of interest may be in a “therapeutic gene” or “therapeutically useful gene.” “Therapeutic genes” are genes where there could be some therapeutic benefit obtained from up- or down-regulating expression, or otherwise altering the structure or function, of that gene.

In some embodiments, the sequence of interest can be positioned upstream of a test promoter for use in the bacterial two-hybrid system (Joung et al., 2000, Proc. Natl. Acad. Sci. USA, 97:7382 and US Patent Application No. 2002/0119498).

### Polypeptide Expression Systems

Once designed, the CoDA engineered Zf proteins described herein can be produced by any means known in the art. For example, a nucleic acid encoding the engineered Zf protein can be produced by synthetic methods.

In some embodiments, a nucleic acid encoding the engineered Zf protein can be produced by recombinant DNA methods from nucleic acids that encode one or more of the engineered Zfs. A variety of in vitro DNA recombination methods exist. Examples include those described in described in U.S. Pat. No. 6,489,145; U.S. Pat. No. 6,395,547; U.S. Pat. No. 5,965,408; and in Horton et al., 1995, Mol. Biotechnol., 3:93-99. Typically, recombination methods depend on a step of making fragments, and a step of recombining the fragments. For example, U.S. Pat. No. 5,605,793 generally relies on fragmentation of double stranded DNA molecules by DNase I. U.S. Pat. No. 5,965,408 generally relies on the annealing of relatively short random primers to target genes and extending them with DNA polymerase. Each of these disclosures relies on polymerase chain reaction (PCR)-like thermocycling of fragments in the presence of DNA polymerase to recombine the fragments.

In order to use the engineered proteins of the present invention, it is typically necessary to express the engineered proteins from a nucleic acid that encodes them. This can be performed in a variety of ways. For example, the nucleic acid encoding the engineered Zf protein is typically cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the engineered Zf protein or production of protein. The nucleic acid encoding the engineered Zf protein is also typically cloned into an expression vector, for administration to a plant cell, animal cell, preferably a mammalian cell or a human cell, fungal cell, bacterial cell, or protozoan cell.

To obtain expression of a cloned gene or nucleic acid, the engineered Zf protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered Zf protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the engineered Zf protein nucleic acid depends on the particular application. For example, a strong constitutive promoter is typically used for expression and purification of the engineered Zf protein. In contrast, when the engineered Zf protein is to be administered in vivo for gene regulation, either a constitutive or an inducible promoter can be used, depending on the particular use of the engineered Zf protein. In addition, a preferred promoter for administration of the engineered Zf protein can be a weak promoter, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the Zf protein signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the engineered Zf protein, e.g., expression in plants, animals, bacteria, fungus, protozoa, etc. Standard bacterial expression vectors include plasmids such as pBR322 based plasmids, pSKF, pET23D, and commercially available fusion expression systems such as GST and LacZ. A preferred fusion protein is the maltose binding protein, “MBP.” Such fusion proteins can be used for purification of the engineered Zf protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include PMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the engineered Zf protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### Characterization of CoDA Engineered Proteins

Engineered Zf proteins designed using methods of the present invention can be further characterized to ensure that they have the desired characteristics for their chosen use. For example, Zfs can be assayed using a bacterial two-hybrid, phage-display, or ribosome display system or using an electrophoretic mobility shift assay or “EMSA” (Buratowski & Chodosh, in Current Protocols in Molecular Biology pp. 12.2.1-12.2.7). Equally, any other DNA binding assay known in the art could be used to verify the DNA binding properties of the selected protein.

In one embodiment, a eukaryotic or prokaryotic cell-based expression system is used. Use of such a cell-based system advantageously provides for the expression of proteins inside living cells, thus the Zf proteins identified are assayed in a cellular context.

In a more preferred embodiment, a bacterial “two-hybrid” system is used to express and test the Zfs of the present invention. The bacterial two-hybrid system has an additional advantage, in that the protein expression and the DNA binding “assay” occur within the same cells, thus there is no separate DNA binding assay to set up.

Methods for the use of the bacterial two-hybrid system to express and assay Zf proteins are described in Joung et al., 2000, Proc. Natl. Acad. Sci. USA, 97:7382, Wright et al., 2006, Nat. Protoc, 1:1637-52; Maeder et al., 2008, Mol. Cell, 31:294-301; Maeder et al., 2009, Nat. Protoc., 4:1471-1501; and US Patent Application No. 2002/0119498, the contents of which are incorporated herein by reference. Briefly, in the bacterial two-hybrid system, the zinc finger protein is expressed in a bacterial strain bearing the sequence of interest upstream of a weak promoter controlling expression of a reporter gene (e.g., histidine 3 (HIS3), the beta-lactamase antibiotic resistance gene, or the beta-galactosidase (lacZ) gene). Expression of the reporter gene occurs in cells in which the zinc finger protein expressed by the cell binds to the target site sequence. Thus, bacterial cells expressing zinc finger proteins that bind to their target site are identified by detection of an activity related to the reporter gene (e.g., growth on selective media, expression of beta-galactosidase). In some embodiments, the Zf proteins activate transcription more than 1.57-fold (e.g., more than 2-fold, more than 2.5-fold, more than 3-fold, more than 3.5-fold, more than 4-fold, more than 5-fold, more than 6-fold, more than 7-fold, more than 8-fold, more than 9-fold, more than 10-fold, more than 12-fold, or more than 15-fold) in a bacterial two-hybrid reporter assay.

In some embodiments, calculations of binding affinity and specificity are also made. This can be done by a variety of methods. The affinity with which the selected Zf protein binds to the sequence of interest can be measured and quantified in terms of its K_{D}. Any assay system can be used, as long is it gives an accurate measurement of the actual K_{D} of the Zf protein. In one embodiment, the K_{D} for the binding of a Zf protein to its target is measured using an EMSA

In one embodiment, EMSA is used to determine the K_{D} for binding of the selected Zf protein both to the sequence of interest (i.e., the specific K_{D}) and to non-specific DNA (i.e., the non-specific K_{D}). Any suitable non-specific or “competitor” double stranded DNA known in the art can be used. In some embodiments, calf thymus DNA or human placental DNA is used. The ratio of the non-specific K_{D} to the specific KD is the specificity ratio. Zfs that bind with high specificity have a high specificity ratio. This measurement is very useful in deciding which of a group of selected Zfs should be used for a given purpose. For example, use of Zfs in vivo requires not only high affinity binding but also high-specificity binding. In a preferred embodiment, Zfs isolated using methods of the present invention have binding specificities higher than Zfs selected using other selection strategies (such as parallel selection and bipartite selection), and even more preferably, comparable or superior to those of naturally occurring multi-finger proteins, such as Zif268.

### Construction of Chimeric Zf Proteins

Often, the aim of producing a custom-designed Zf DNA binding domain by CoDA is to obtain a Zf protein that can be used to perform a function. The Zf DBD can be used alone, for example to bind to a specific site on a gene and thus block binding of other DNA-binding domains. However, in some embodiments, the Zf will be used in the construction of a chimeric Zf protein containing a Zf DNA binding domain and an additional domain having some desired specific function (e.g., gene activation) or enzymatic activity i.e., a “functional domain.”

Chimeric Zf proteins designed and produced using the methods described herein can be used to perform any function where it is desired to target, for example, some specific enzymatic activity to a specific DNA sequence, as well as any of the functions already described for other types of synthetic or engineered zinc finger molecules. Engineered Zf DNA binding domains, can be used in the construction of chimeric proteins useful for the treatment of disease (see, for example, U.S. patent application 2002/0160940, and U.S. Pat. Nos. 6,511,808, 6,013,453 and 6,007,988, and International patent application WO 02/057308), or for otherwise altering the structure or function of a given gene in vivo. The engineered Zf proteins of the present invention are also useful as research tools, for example, in performing either in vivo or in vitro functional genomics studies (see, for example, U.S. Pat. No. 6,503,717 and U.S. patent application 2002/0164575).

To generate a functional recombinant protein, the engineered Zf DNA binding domain will typically be fused to at least one “functional” domain. Fusing functional domains to synthetic Zf proteins to form functional transcription factors involves only routine molecular biology techniques which are commonly practiced by those of skill in the art, see for example, U.S. Pat. Nos. 6,511,808, 6,013,453, 6,007,988, 6,503,717 and U.S. patent application 2002/0160940).

Functional domains can be associated with the engineered Zf domain at any suitable position, including the C- or N-terminus of the Zf protein. Suitable “functional” domains for addition to the engineered protein made using the methods of the invention are described in U.S. Pat. Nos. 6,511,808, 6,013,453, 6,007,988, and 6,503,717 and U.S. patent application 2002/0160940.

In one embodiment, the functional domain is a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. It is preferred that a nuclear localization domain is routinely incorporated into the final chimeric protein, as the ultimate functions of the chimeric proteins of the present invention will typically require the proteins to be localized in the nucleus. However, it may not be necessary to add a separate nuclear localization domain in cases where the engineered Zf domain itself, or another functional domain within the final chimeric protein, has intrinsic nuclear translocation function.

In another embodiment, the functional domain is a transcriptional activation domain such that the chimeric protein can be used to activate transcription of the gene of interest. Any transcriptional activation domain known in the art can be used, such as for example, the VP16 domain form herpes simplex virus (Sadowski et al., 1988, Nature, 335:563-564) or the p65 domain from the cellular transcription factor NF-kappaB (Ruben et al., 1991, Science, 251:1490-93).

In yet another embodiment, the functional domain is a transcriptional repression domain such that the chimeric protein can be used to repress transcription of the gene of interest. Any transcriptional repression domain known in the art can be used, such as for example, the KRAB (Kruppel-associated box) domain found in many naturally occurring KRAB proteins (Thiesen et al., 1991, Nucleic Acids Res., 19:3996).

In a further embodiment, the functional domain is a DNA modification domain such as a methyltransferase (or methylase) domain, a de-methylation domain, an acetylation domain, or a deacetylation domain. Many such domains are known in the art and any such domain can be used, depending on the desired function of the resultant chimeric protein. For example, it has been shown that a DNA methylation domain can be fused to a Zf protein and used for targeted methylation of a specific DNA sequence (Xu et al., 1997, Nat. Genet., 17:376-378). The state of methylation of a gene affects its expression and regulation, and furthermore, there are several diseases associated with defects in DNA methylation.

In a still further embodiment the functional domain is a chromatin modification domain such as a histone acetylase or histone de-acetylase (or HDAC) domain. Many such domains are known in the art and any such domain can be used, depending on the desired function of the resultant chimeric protein. Histone deacetylases (such as HDAC1 and HDAC2) are involved in gene repression. Therefore, by targeting HDAC activity to a specific gene of interest using an engineered Zf protein, the expression of the gene of interest can be repressed.

In an alternative embodiment, the functional domain is a nuclease domain, such as a restriction endonuclease (or restriction enzyme) domain. The DNA cleavage activity of a nuclease enzyme can be targeted to a specific target sequence by fusing it to an appropriate engineered Zf DNA binding domain. In this way, sequence specific chimeric restriction enzyme can be produced. Several nuclease domains are known in the art and any suitable nuclease domain can be used. For example, an endonuclease domain of a type II restriction endonuclease (e.g., FokI) can be used, as taught be Kim et al., 1996, Proc. Natl. Acad. Sci. USA, 6:1156-60). In some embodiments, the endonuclease is an engineered FokI variant as described in US 2008/0131962. Such chimeric endonucleases can be used in any situation where cleavage of a specific DNA sequence is desired, such as in laboratory procedures for the construction of recombinant DNA molecules, or in producing double-stranded DNA breaks in genomic DNA in order to promote homologous recombination (Kim et al., 1996, Proc. Natl. Acad. Sci. USA, 6:1156-60; Bibikova et al., 2001, Mol. Cell. Biol., 21:289-297; Porteus & Baltimore, 2003, Science, 300:763). Repair of zinc finger nuclease-induced double-strand breaks (DSB) by error-prone non-homologous end joining leads to efficient introduction of insertion or deletion mutations at the site of the DSB (Bibikova et al., 2002, Genetics, 161:1169-75). Alternatively, repair of a DSB by homology-directed repair with an exogenously introduced “donor template” can lead to highly efficient introduction of precise base alterations or insertions at the break site (Bibikova et al., 2003, Science, 300:764; Urnov et al., 2005, Nature, 435:646-651; Porteus et al., 2003, Science, 300:763).

In some embodiments, the functional domain is an integrase domain, such that the chimeric protein can be used to insert exogenous DNA at a specific location in, for example, the human genome.

Other suitable functional domains include silencer domains, nuclear hormone receptors, resolvase domains oncogene transcription factors (e.g., myc, jun, fos, myb, max, mad, rel, ets, bcl, myb, mos family members etc), kinases, phosphatases, and any other proteins that modify the structure of DNA and/or the expression of genes. Suitable kinase domains, from kinases involved in transcription regulation are reviewed in Davis, 1995, Mol. Reprod. Dev., 42:459-67. Suitable phosphatase domains are reviewed in, for example, Schonthal & Semin, 1995, Cancer Biol. 6:239-48.

Fusions of CoDA Zfs to functional domains can be performed by standard recombinant DNA techniques well known to those skilled in the art, and as are described in, for example, basic laboratory texts such as Sambrook et al., Molecular Cloning; A Laboratory Manual 3d ed. (2001), and in U.S. Pat. Nos. 6,511,808, 6,013,453, 6,007,988, and 6,503,717 and U.S. patent application 2002/0160940.

In some embodiments, the DNA binding domain used to form the recombinant proteins of the present invention is the exact CoDA engineered protein that has been designed.

In some embodiments, two or more engineered Zf proteins are linked together to produce the final DNA binding domain. The linkage of two or more engineered proteins may be performed by covalent or non-covalent means. In the case of covalent linkage, engineered proteins can be covalently linked together using an amino acid linker (see, for example, U.S. patent application 2002/0160940, and International applications WO 02/099084 and WO 01/53480). This linker may be any string of amino acids desired. In one embodiment the linker is a canonical TGEKP linker. Whatever linkers are used, standard recombinant DNA techniques (such as described in, for example, Sambrook et al., Molecular Cloning; A Laboratory Manual 3d ed. (2001)) can be used to produce such linked proteins.

In the case of non-covalent linkage, two or more engineered proteins may be multimerized, i.e., two or more folded engineered protein “subunits” may associate with each other by non-covalent interactions to form a “multi-subunit protein assembly” or “multimeric complex”. Where only two engineered proteins are non-covalently linked, the proteins are said to be dimerized. In one embodiment two identical engineered proteins may be linked to form a homo-dimer. In an alternative embodiment two different engineered proteins may be linked to form a hetero-dimer. For example, a six-finger protein may be produced by dimerization of two three-finger proteins, or an eight-finger protein may be produced by dimerization of two four-finger proteins. The production of multimers or dimers can be performed by fusing “multimerization” or “dimerization domains” to the zinc finger proteins to be joined. Any suitable method for fusing protein domains or producing chimeric proteins can be used. For example, in one embodiment, the DNA encoding the zinc finger protein is fused to the DNA encoding the multimerization domain using standard recombinant DNA techniques (as described in, for example, Sambrook et al., Molecular Cloning; A Laboratory Manual 3d ed. (2001)).

Suitable multimerization or dimerization domains can be selected from any protein that is known to exists as a multimer or dimer, or any protein known to possess such multimerization or dimerization activity. Examples, of suitable domains include the dimerization element of Gal4, leucine zipper domains, STAT protein N-terminal domains, FK506 binding proteins, and randomized peptides selected for Zf dimerization activity (see, e.g., Bryan et al., 1999, Proc. Natl. Acad. Sci. USA, 96:9568; Pomerantz et al., 1998, Biochemistry, 37:965-970; Wolfe et al., 2000, Structure, 8: 739-750; O'Shea, 1991, Science, 254:539; Barahmand-Pour et al., 1996, Curr. Top. Microbiol. Immunol., 211:121-128; Klemm et al., 1998, Annu Rev. Immunol., 16:569-592; Ho et al., 1996, Nature, 382:822-826). Furthermore, some zinc finger proteins themselves have dimerization activity. For example, the zinc fingers from the transcription factor Ikaros have dimerization activity (McCarty et al., 2003, Mol. Cell, 11:459-470). Thus, if the engineered Zf proteins themselves have dimerization function there will be no need to fuse an additional dimerization domain to these proteins. In certain embodiments, “conditional” multimerization of dimerization” technology can be used. For example, this can be accomplished using FK506 and FKBP interactions. FK506 binding domains are attached to the proteins to be dimerized. These proteins will remain apart in the absence of a dimerizer. Upon addition of a dimerizer, such as the synthetic ligand FK1012, the two proteins will fuse.

In embodiments where the engineered proteins are used in the generation of chimeric endonuclease it is preferred that the chimeric protein possesses a dimerization domain as such endonucleases are believed to function as dimers. Any suitable dimerization domain may be used. In one embodiment the endonuclease domain itself possesses dimerization activity. For example, the nuclease domain of Fok I which has intrinsic dimerization activity can be used (Kim et al., 1996, Proc. Natl. Acad. Sci., 93:1156-60).

### Assays for Determining Regulation of Gene Expression by Engineered Proteins

A variety of assays can be used to determine the level of gene expression regulation by the engineered Zf proteins, see for example U.S. Pat. No. 6,453,242. The activity of a particular engineered Zf protein can be assessed using a variety of in vitro and in vivo assays, by measuring, e.g., protein or mRNA levels, product levels, enzyme activity, tumor growth; transcriptional activation or repression of a reporter gene; second messenger levels (e.g., cGMP, cAMP, IP3, DAG, Ca²⁺); cytokine and hormone production levels; and neovascularization, using, e.g., immunoassays (e.g., ELISA and immunohistochemical assays with antibodies), hybridization assays (e.g., RNase protection, northerns, in situ hybridization, oligonucleotide array studies), colorimetric assays, amplification assays, enzyme activity assays, tumor growth assays, phenotypic assays, and the like.

CoDA engineered Zf proteins can be first tested for activity in vitro using cultured cells, e.g., 293 cells, CHO cells, VERO cells, BHK cells, HeLa cells, COS cells, and the like. In some embodiments, human cells are used. The engineered Zf protein is often first tested using a transient expression system with a reporter gene, and then regulation of the target endogenous gene is tested in cells and in animals, both in vivo and ex vivo. The engineered Zf protein can be recombinantly expressed in a cell, recombinantly expressed in cells transplanted into an animal, or recombinantly expressed in a transgenic animal, as well as administered as a protein to an animal or cell using delivery vehicles described below. The cells can be immobilized, be in solution, be injected into an animal, or be naturally occurring in a transgenic or non-transgenic animal.

Modulation of gene expression is tested using one of the in vitro or in vivo assays described herein. Samples or assays are treated with the engineered Zf protein and compared to un-treated control samples, to examine the extent of modulation. For regulation of endogenous gene expression, the CoDA Zf protein ideally has a K_{D} of 200 nM or less, more preferably 100 nM or less, more preferably 50 nM, most preferably 25 nM or less. The effects of the engineered Zf protein can be measured by examining any of the parameters described above. Any suitable gene expression, phenotypic, or physiological change can be used to assess the influence of the engineered Zf protein. When the functional consequences are determined using intact cells or animals, one can also measure a variety of effects such as tumor growth, neovascularization, hormone release, transcriptional changes to both known and uncharacterized genetic markers (e.g., northern blots or oligonucleotide array studies), changes in cell metabolism such as cell growth or pH changes, and changes in intracellular second messengers such as cGMP.

Preferred assays for regulation of endogenous gene expression can be performed in vitro. In one in vitro assay format, the engineered Zf protein regulation of endogenous gene expression in cultured cells is measured by examining protein production using an ELISA assay. The test sample is compared to control cells treated with an empty vector or an unrelated Zf protein that is targeted to another gene.

In another embodiment, regulation of endogenous gene expression is determined in vitro by measuring the level of target gene mRNA expression. The level of gene expression is measured using amplification, e.g., using RT-PCR, LCR, or hybridization assays, e.g., northern hybridization, RNase protection, dot blotting. RNase protection is used in one embodiment. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the target gene promoter operably linked to a reporter gene such as luciferase, green fluorescent protein, CAT, or beta-galactosidase. The reporter construct is typically co-transfected into a cultured cell. After treatment with the engineered Zf protein, the amount of reporter gene transcription, translation, or activity is measured according to standard techniques known to those of skill in the art.

Another example of an assay format useful for monitoring regulation of endogenous gene expression is performed in vivo. This assay is particularly useful for examining Zf proteins that inhibit expression of tumor promoting genes, genes involved in tumor support, such as neovascularization (e.g., VEGF), or that activate tumor suppressor genes such as p53. In this assay, cultured tumor cells expressing the engineered Zf protein are injected subcutaneously into an immune compromised mouse such as an athymic mouse, an irradiated mouse, or a SCID mouse. After a suitable length of time, preferably 4-8 weeks, tumor growth is measured, e.g., by volume or by its two largest dimensions, and compared to the control. Tumors that have statistically significant reduction (using, e.g., Student's T test) are said to have inhibited growth. Alternatively, the extent of tumor neovascularization can also be measured. Immunoassays using endothelial cell specific antibodies are used to stain for vascularization of the tumor and the number of vessels in the tumor. Tumors that have a statistically significant reduction in the number of vessels (using, e.g., Student's T test) are said to have inhibited neovascularization.

Transgenic and non-transgenic animals can also be used for examining regulation of endogenous gene expression in vivo. Transgenic animals typically express the engineered Zf protein. Alternatively, animals that transiently express the engineered Zf protein, or to which the engineered Zf protein has been administered in a delivery vehicle, can be used. Regulation of endogenous gene expression is tested using any one of the assays described herein.

### Use of Engineered Zf Proteins in Gene Therapy

The engineered proteins of the present invention can be used to regulate gene expression or alter gene sequence in gene therapy applications in the same as has already been described for other types of synthetic zinc finger proteins, see for example U.S. Pat. No. 6,511,808, U.S. Pat. No. 6,013,453, U.S. Pat. No. 6,007,988, U.S. Pat. No. 6,503,717, U.S. patent application 2002/0164575, and U.S. patent application 2002/0160940.

Conventional viral and non-viral based gene transfer methods can be used to introduce nucleic acids encoding the engineered Zf protein into mammalian cells or target tissues. Such methods can be used to administer nucleic acids encoding engineered Zf proteins to cells in vitro. Preferably, the nucleic acids encoding the engineered Zf protein s are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, 1992, Science, 256:808-813; Nabel & Felgner, 1993, TIBTECH, 11:211-217; Mitani & Caskey, 1993, TIBTECH, 11:162-166; Dillon, 1993, TIBTECH, 11:167-175; Miller, 1992, Nature, 357:455-460; Van Brunt, 1988, Biotechnology, 6:1149-54; Vigne, 1995, Restorat. Neurol. Neurosci., 8:35-36; Kremer & Perricaudet, 1995, Br. Med. Bull., 51:31-44; Haddada et al., in Current Topics in Microbiology and Immunology Doerfler and Bohm (eds) (1995); and Yu et al., 1994, Gene Ther., 1:13-26.

Methods of non-viral delivery of nucleic acids encoding the engineered Zf proteins include lipofection, microinjection, biolistics, virosomes, liposomes, immunoliposomes, polycation or lipid:nucleic acid conjugates, naked DNA or RNA, artificial virions, and agent-enhanced uptake of DNA or RNA. Lipofection is described in e.g., U.S. Pat. No. 5,049,386, No. 4,946,787; and No.4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, 1995, Science, 270:404-410; Blaese et al., 1995, Cancer Gene Ther., 2:291-297; Behr et al., 1994, Bioconjugate Chem. 5:382-389; Remy et al., 1994, Bioconjugate Chem., 5:647-654; Gao et al., Gene Ther., 2:710-722; Ahmad et al., 1992, Cancer Res., 52:4817-20; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding the engineered Zf proteins takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of Zf proteins could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992, J. Virol., 66:2731-39; Johann et al., 1992, J. Virol., 66:1635-40; Sommerfelt et al., 1990, Virololgy, 176:58-59; Wilson et al., 1989, J. Virol., 63:2374-78; Miller et al., 1991, J. Virol., 65:2220-24; WO 94/26877).

In applications where transient expression of the engineered Zf protein is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987, Virology 160:38-47; U.S. Pat. No. 4,797,368; WO 93/24641; Kotin, 1994, Hum. Gene Ther., 5:793-801; Muzyczka, 1994, J. Clin. Invest., 94:1351). Construction of recombinant AAV vectors are described in a number of publications, including U.S. Pat. No. 5,173,414; Tratschin et al., 1985, Mol. Cell. Biol. 5:3251-60; Tratschin et al.,1984, Mol. Cell. Biol., 4:2072-81; Hermonat & Muzyczka, 1984, Proc. Natl. Acad. Sci. USA, 81:6466-70; and Samulski et al., 1989, J. Virol., 63:3822-28.

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., 1995, Blood, 85:3048; Kohn et al.,1995, Nat. Med., 1:1017; Malech et al., 1997, Proc. Natl. Acad. Sci. USA, 94:12133-38). PA317/pLASN was the first therapeutic vector used in a gene therapy trial. (Blaese et al., 1995, Science, 270:475-480). Transduction efficiencies of 50% or greater have been observed for MFG-S packaged vectors (Ellem et al., 1997, Immunol Immunother., 44:10-20; Dranoffet al., 1997, Hum. Gene Ther., 1:111-112).

Recombinant adeno-associated virus vectors (rAAV) are a promising alternative gene delivery systems based on the defective and nonpathogenic parvovirus adeno-associated type 2 virus. Typically, the vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Lancet, 351:1702-1703; Kearns et al., 1996, Gene Ther., 9:748-55).

Replication-deficient recombinant adenoviral vectors (Ad) are predominantly used for colon cancer gene therapy, because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., 1998, Hum. Gene Ther. 7:1083-89). Additional examples of the use of adenovirus vectors for gene transfer in clinical trials include Rosenecker et al., 1996, Infection, 24:15-10; Sterman et al., 1998, Hum. Gene Ther., 9:7 1083-89; Welsh et al., 1995, Hum. Gene Ther., 2:205-218; Alvarez et al., 1997, Hum. Gene Ther. 5:597-613; Topf et al., 1998, Gene Ther., 5:507-513; Sterman et al., 1998, Hum. Gene Ther., 7:1083-89.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, Proc. Natl. Acad. Sci. USA, 92:9747-51, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or stem cells (e.g., universal donor hematopoietic stem cells, embryonic stem cells (ES), partially differentiated stem cells, non-pluripotent stem cells, pluripotent stem cells, induced pluripotent stem cells (iPS cells) (see e.g., Sipione et al., Diabetologia, 47:499-508, 2004)), followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with nucleic acid (gene or cDNA), encoding the engineered Zf protein, and re-infused back into the subject organism (e.g., patient). Various cell types suitable for ex vivo transfection are well known to those of skill in the art (see, e.g., Freshney et al., Culture of Animal Cells, A Manual of Basic Technique (5th ed. 2005)) and the references cited therein for a discussion of how to isolate and culture cells from patients).

In one embodiment, stem cells (e.g., universal donor hematopoietic stem cells, embryonic stem cells (ES), partially differentiated stem cells, non-pluripotent stem cells, pluripotent stem cells, induced pluripotent stem cells (iPS cells) (see e.g., Sipione et al., Diabetologia, 47:499-508, 2004)) are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma and TNF-alpha are known (see Inaba et al., 1992, J. Exp. Med., 176:1693-1702).

Stem cells can be isolated for transduction and differentiation using known methods. For example, stem cells can be isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., 1992, J. Exp. Med., 176:1693-1702).

Vectors (e.g., retroviruses, adenoviruses, liposomes, etc.) containing the engineered Zf protein nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Alternatively, stable formulations of the engineered Zf protein can also be administered.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington: The Science and Practice of Pharmacy, 21st ed., 2005).

### Delivery Vehicles

An important factor in the administration of polypeptide compounds, such as the engineered Zf proteins of the present invention, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as engineered Zf protein across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Curr. Opin. Neurobiol., 6:629-634). Another subsequence, the h (hydrophobic) domain of signal peptides, was found to have similar cell membrane translocation characteristics (see, e.g., Lin et al., 1995, J. Biol. Chem., 270:14255-58).

Examples of peptide sequences that can be linked to a protein, for facilitating uptake of the protein into cells, include, but are not limited to: peptide fragments of the tat protein of HIV (Endoh et al., 2010, Methods Mol. Biol., 623:271-281; Schmidt et al., 2010, FEBS Lett., 584:1806-13; Futaki, 2006, Biopolymers, 84:241-249); a 20 residue peptide sequence which corresponds to amino acids 84-103 of the p16 protein (see Fahraeus et al., 1996, Curr. Biol., 6:84); the third helix of the 60-amino acid long homeodomain of Antennapedia (Derossi et al., 1994, J. Biol. Chem., 269:10444); the h region of a signal peptide, such as the Kaposi fibroblast growth factor (K-FGF) h region (Lin et al., supra); or the VP22 translocation domain from HSV (Elliot & O'Hare, 1997, Cell, 88:223-233). See also, e.g., Caron et al., 2001, Mol Ther., 3:310-318; Langel, Cell-Penetrating Peptides: Processes and Applications (CRC Press, Boca Raton FL 2002); El-Andaloussi et al., 2005, Curr. Pharm. Des., 11:3597-3611; and Deshayes et al., 2005, Cell. Mol. Life Sci., 62:1839-49. Other suitable chemical moieties that provide enhanced cellular uptake may also be chemically linked to the CSPO-selected Zf proteins of the present invention.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., 1993, J. Biol. Chem., 268:3334-41; Perelle et al., 1993, Infect. Immun., 61:5147-56; Stenmark et al., 1991, J. Cell Biol., 113:1025-32; Donnelly et al., 1993, Proc. Natl. Acad. Sci. USA, 90:3530-34; Carbonetti et al., 1995, Abstr. Annu Meet. Am. Soc. Microbiol. 95:295; Sebo et al., 1995, Infect. Immun., 63:3851-57; Klimpel et al., 1992, Proc. Natl. Acad. Sci. USA, 89:10277-81; and Novak et al., 1992, J. Biol. Chem., 267:17186-93).

Such subsequences can be used to translocate engineered Zf proteins across a cell membrane. The engineered Zf proteins can be conveniently fused to or derivatized with such sequences. Typically, the translocation sequence is provided as part of a fusion protein. Optionally, a linker can be used to link the engineered Zf protein and the translocation sequence. Any suitable linker can be used, e.g., a peptide linker.

The engineered Zf protein can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., the engineered Zf protein.

The liposome fuses with the plasma membrane, thereby releasing the compound into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, the engineered Zf protein) at the target tissue or cell. For systemic or tissue specific delivery, this can be accomplished, for example, in a passive manner wherein the liposome bilayer degrades over time through the action of various agents in the body. Alternatively, active compound release involves using an agent to induce a permeability change in the liposome vesicle. Liposome membranes can be constructed so that they become destabilized when the environment becomes acidic near the liposome membrane (see, e.g., Proc. Natl. Acad. Sci. USA, 84:7851 (1987); Biochemistry, 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise the engineered Zf protein and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., 1980, Annu Rev. Biophys. Bioeng., 9:467, U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication. No. WO 91/17424, Deamer & Bangham, 1976, Biochim. Biophys. Acta, 443:629-634; Fraley, et al., 1979, Proc. Natl. Acad. Sci. USA, 76:3348-52; Hope et al., 1985, Biochim. Biophys. Acta, 812:55-65; Mayer et al., 1986, Biochim. Biophys. Acta, 858:161-168; Williams et al., 1988, Proc. Natl. Acad. Sci. USA, 85:242-246; Liposomes (Ostro (ed.), 1983, Chapter 1); Hope et al., 1986, Chem. Phys. Lip., 40:89; Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., 1990, J. Biol. Chem., 265:16337-42 and Leonetti et al., 1990, Proc. Natl. Acad. Sci. USA, 87:2448-51).

### Dosages

For therapeutic applications, the dose of the engineered Zf protein to be administered to a patient is calculated in the same way as has already been described for other types of synthetic zinc finger proteins, see for example U.S. Pat. No. 6,511,808, U.S. Pat. No. 6,492,117, U.S. Pat. No. 6,453,242, U.S. patent application 2002/0164575, and U.S. patent application 2002/0160940. In the context of the present disclosure, the dose should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy, specificity, and K_{D} of the particular engineered Zf protein employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

### Pharmaceutical Compositions and Administration

Appropriate pharmaceutical compositions for administration of the engineered Zf proteins of the present invention are determined as already described for other types of synthetic zinc finger proteins, see for example U.S. Pat. No. 6,511,808, U.S. Pat. No. 6,492,117, U.S. Pat. No. 6,453,242, U.S. patent application 2002/0164575, and U.S. patent application 2002/0160940. Engineered Zf proteins, and expression vectors encoding engineered Zf proteins, can be administered directly to the patient for modulation of gene expression and for therapeutic or prophylactic applications, for example, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, and the like. Examples of microorganisms that can be inhibited by Zf gene therapy include pathogenic bacteria, e.g., chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme disease bacteria; infectious fungus, e.g., Aspergillus, Candida species; protozoa such as sporozoa (e.g., Plasmodia), rhizopods (e.g., Entamoeba) and flagellates (Trypanosoma, Leishmania, Trichomonas, Giardia, etc.); viral diseases, e.g., hepatitis (A, B, or C), herpes virus (e.g., VZV, HSV-1, HSV-6, HSV-II, CMV, and EBV), HIV, Ebola, adenovirus, influenza virus, flaviviruses, echovirus, rhinovirus, coxsackie virus, comovirus, respiratory syncytial virus, mumps virus, rotavirus, measles virus, rubella virus, parvovirus, vaccinia virus, HTLV virus, dengue virus, papillomavirus, poliovirus, rabies virus, and arboviral encephalitis virus, etc.

Administration of therapeutically effective amounts is by any of the routes normally used for introducing Zf proteins into ultimate contact with the tissue to be treated. The Zf proteins are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington: The Science and Practice of Pharmacy, 21st ed., 2005).

The engineered Zf proteins, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

### Use of Zinc Finger Nucleases

Zinc finger nucleases engineered using the methods described herein can be used to induce mutations in a genomic sequence, e.g., by cleaving at two sites and deleting sequences in between, by cleavage at a single site followed by non-homologous end joining, and/or by cleaving at a site so as to remove one or two or a few nucleotides. In some embodiments, the zinc finger nuclease is used to induce mutation in an animal, plant, fungal, or bacterial genome. Targeted cleavage can also be used to create gene knock-outs (e.g., for functional genomics or target validation) and to facilitate targeted insertion of a sequence into a genome (i.e., gene knock-in); e.g., for purposes of cell engineering or protein overexpression. Insertion can be by means of replacements of chromosomal sequences through homologous recombination or by targeted integration, in which a new sequence (i.e., a sequence not present in the region of interest), flanked by sequences homologous to the region of interest in the chromosome, is inserted at a predetermined target site. Exogenous DNA can also be inserted into ZFN-induced double stranded breaks without the need for flanking homology sequences (see, Orlando et al., 2010, Nucl. Acids Res., 1-15, doi:10.1093/nar/gkq512).

The same methods can also be used to replace a wild-type sequence with a mutant sequence, or to convert one allele to a different allele.

Targeted cleavage of infecting or integrated viral genomes can be used to treat viral infections in a host. Additionally, targeted cleavage of genes encoding receptors for viruses can be used to block expression of such receptors, thereby preventing viral infection and/or viral spread in a host organism. Targeted mutagenesis of genes encoding viral receptors (e.g., the CCR5 and CXCR4 receptors for HIV) can be used to render the receptors unable to bind to virus, thereby preventing new infection and blocking the spread of existing infections. Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, etc.); Caliciviridae; Togaviridae (e.g., rubella virus, dengue virus, etc.); Flaviviridae; Coronaviridae; Reoviridae; Bimaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses. See, e.g., Virology, 3rd Edition (W. K. Joklik, ed. 1988); Fundamental Virology, 4th Edition (Knipe and Howley, eds. 2001), for a description of these and other viruses. Receptors for HIV, for example, include CCR-5 and CXCR-4.

In similar fashion, the genome of an infecting bacterium can be mutagenized by targeted DNA cleavage followed by non-homologous end joining, to block or ameliorate bacterial infections.

The disclosed methods for targeted recombination can be used to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods of targeted recombination disclosed herein.

Exemplary genetic diseases include, but are not limited to, achondroplasia, achromatopsia, acid maltase deficiency, adenosine deaminase deficiency (OMIM No.102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, Fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefelter's syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted DNA cleavage and/or homologous recombination include acquired immunodeficiencies, lysosomal storage diseases (e.g., Gaucher's disease, GM1, Fabry disease and Tay-Sachs disease), mucopolysaccahidosis (e.g., Hunter's disease, Hurler's disease), hemoglobinopathies (e.g., sickle cell diseases, HbC, alpha-thalassemia, beta-thalassemia) and hemophilias.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or by replacing its control sequences with sequences that support a lower, non-pathologic level of expression. As another example, the wild-type allele of the ApoAI gene can be replaced by the ApoAI Milano allele, to treat atherosclerosis. Indeed, any pathology dependent upon a particular genomic sequence, in any fashion, can be corrected or alleviated using the methods and compositions disclosed herein.

Targeted cleavage and targeted recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

The compositions and methods described herein also allow for novel approaches and systems to address immune reactions of a host to allogeneic grafts. In particular, a major problem faced when allogeneic stem cells (or any type of allogeneic cell) are grafted into a host recipient is the high risk of rejection by the host's immune system, primarily mediated through recognition of the Major Histocompatibility Complex (MHC) on the surface of the engrafted cells. The MHC comprises the HLA class I protein(s) that function as heterodimers that are comprised of a common beta subunit and variable alpha subunits. It has been demonstrated that tissue grafts derived from stem cells that are devoid of HLA escape the host's immune response. See, e.g., Coffman et al., 1993, J. Immunol., 151:425-35; Markmann et al., 1992, Transplantation, 54:1085-89; Koller et al., 1990, Science, 248:1227-30. Using the compositions and methods described herein, genes encoding HLA proteins involved in graft rejection can be cleaved, mutagenized or altered by recombination, in either their coding or regulatory sequences, so that their expression is blocked or they express a non-functional product. For example, by inactivating the gene encoding the common beta subunit gene (beta2 microglobulin) using ZFP fusion proteins as described herein, HLA class I can be removed from the cells to rapidly and reliably generate HLA class I null stem cells from any donor, thereby reducing the need for closely matched donor/recipient MHC haplotypes during stem cell grafting.

Inactivation of any gene (e.g., the beta2 microglobulin gene) can be achieved, for example, by a single cleavage event, by cleavage followed by non-homologous end joining, by cleavage at two sites followed by joining so as to delete the sequence between the two cleavage sites, by targeted recombination of a missense or nonsense codon into the coding region, or by targeted recombination of an irrelevant sequence (i.e., a “stuffer” sequence) into the gene or its regulatory region, so as to disrupt the gene or regulatory region.

Targeted modification of chromatin structure, as disclosed in WO 01/83793, can be used to facilitate the binding of fusion proteins to cellular chromatin.

In additional embodiments, one or more fusions between a zinc finger binding domain and a recombinase (or functional fragment thereof) can be used, in addition to or instead of the zinc finger-cleavage domain fusions disclosed herein, to facilitate targeted recombination. See, for example, co-owned U.S. Pat. No. 6,534,261 and Akopian et al. (2003) Proc. Natl. Acad. Sci. USA 100:8688-8691.

In additional embodiments, the disclosed methods and compositions are used to provide fusions of ZFP binding domains with transcriptional activation or repression domains that require dimerization (either homodimerization or heterodimerization) for their activity. In these cases, a fusion polypeptide comprises a zinc finger binding domain and a functional domain monomer (e.g., a monomer from a dimeric transcriptional activation or repression domain). Binding of two such fusion polypeptides to properly situated target sites allows dimerization so as to reconstitute a functional transcription activation or repression domain.

### Regulation of Gene Expression in Plants

Engineered Zf proteins can be used to engineer plants for traits such as increased disease resistance, modification of structural and storage polysaccharides, flavors, proteins, and fatty acids, fruit ripening, yield, color, nutritional characteristics, improved storage capability, and the like. In particular, the engineering of crop species for enhanced oil production, e.g., the modification of the fatty acids produced in oilseeds, is of interest.

Seed oils are composed primarily of triacylglycerols (TAGs), which are glycerol esters of fatty acids. Commercial production of these vegetable oils is accounted for primarily by six major oil crops (soybean, oil palm, rapeseed, sunflower, cotton seed, and peanut). Vegetable oils are used predominantly (90%) for human consumption as margarine, shortening, salad oils, and frying oil. The remaining 10% is used for non-food applications such as lubricants, oleochemicals, biofuels, detergents, and other industrial applications.

The desired characteristics of the oil used in each of these applications varies widely, particularly in terms of the chain length and number of double bonds present in the fatty acids making up the TAGs. These properties are manipulated by the plant in order to control membrane fluidity and temperature sensitivity. The same properties can be controlled using CoDA Zf protein to produce oils with improved characteristics for food and industrial uses.

The primary fatty acids in the TAGs of oilseed crops are 16 to 18 carbons in length and contain 0 to 3 double bonds. Palmitic acid (16:0 [16 carbons: 0 double bonds]), oleic acid (18:1), linoleic acid (18:2), and linolenic acid (18:3) predominate. The number of double bonds, or degree of saturation, determines the melting temperature, reactivity, cooking performance, and health attributes of the resulting oil.

The enzyme responsible for the conversion of oleic acid (18:1) into linoleic acid (18:2) (which is then the precursor for 18:3 formation) is delta-12-oleate desaturase, also referred to as omega-6 desaturase. A block at this step in the fatty acid desaturation pathway should result in the accumulation of oleic acid at the expense of polyunsaturates.

In one embodiment engineered Zf proteins are used to regulate expression of the FAD2-1 gene in soybeans. Two genes encoding microsomal delta-6 desaturases have been cloned recently from soybean, and are referred to as FAD2-1 and FAD2-2 (Heppard et al., 1996, Plant Physiol. 110:311-319). FAD2-1 (delta-12 desaturase) appears to control the bulk of oleic acid desaturation in the soybean seed. Engineered Zf proteins can thus be used to modulate gene expression of FAD2-1 in plants. Specifically, engineered Zf proteins can be used to inhibit expression of the FAD2-1 gene in soybean in order to increase the accumulation of oleic acid (18:1) in the oil seed. Moreover, engineered Zf proteins can be used to modulate expression of any other plant gene, such as delta-9 desaturase, delta-12 desaturases from other plants, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, polygalacturonase, EPSP synthase, plant viral genes, plant fungal pathogen genes, and plant bacterial pathogen genes.

Recombinant DNA vectors suitable for transformation of plant cells are also used to deliver protein (e.g., engineered Zf proteins)-encoding nucleic acids to plant cells. Techniques for transforming a wide variety of higher plant species are well known and described in the technical and scientific literature (see, e.g., Weising et al., 1988, Ann. Rev. Genet., 22:421-477). A DNA sequence coding for the desired Zf protein is combined with transcriptional and translational initiation regulatory sequences which will direct the transcription of the Zf protein in the intended tissues of the transformed plant.

For example, a plant promoter fragment may be employed which will direct expression of the engineered Zf protein in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35 S transcription initiation region, the 1′- or 2′-promoter derived from T-DNA of Agrobacterium tumefaciens, and other transcription initiation regions from various plant genes known to those of skill.

Alternatively, the plant promoter may direct expression of the engineered Zf protein in a specific tissue or may be otherwise under more precise environmental or developmental control. Such promoters are referred to here as “inducible” promoters. Examples of environmental conditions that may effect transcription by inducible promoters include anaerobic conditions or the presence of light.

Examples of promoters under developmental control include promoters that initiate transcription only in certain tissues, such as fruit, seeds, or flowers. For example, the use of a polygalacturonase promoter can direct expression of the Zf protein in the fruit, a CHS-A (chalcone synthase A from petunia) promoter can direct expression of the ZFP in flower of a plant.

The vector comprising the Zf protein sequences will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Such DNA constructs may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al., 1984, EMBO J., 3:2717-22. Electroporation techniques are described in Fromm et al. 1985, Proc. Natl. Acad. Sci. USA, 82:5824. Biolistic transformation techniques are described in Klein et al., 1987, Nature, 327:70-73.

Agrobacterium tumefaciens-meditated transformation techniques are well described in the scientific literature (see, e.g., Horsch et al., 1984, Science, 233:496-498; and Fraley et al., 1983, Proc. Natl. Acad. Sci. USA, 80:4803).

Transformed plant cells which are derived by any of the above transformation techniques can be cultured to regenerate a whole plant which possesses the transformed genotype and thus the desired Zf protein-controlled phenotype. Such regeneration techniques rely on manipulation of certain phytohormones in a tissue culture growth medium, typically relying on a biocide and/or herbicide marker which has been introduced together with the Zf protein nucleotide sequences. Plant regeneration from cultured protoplasts is described in Evans et al., Protoplasts Isolation and Culture, Handbook of Plant Cell Culture, pp. 124-176 (1983); and Binding, Regeneration of Plants, Plant Protoplasts, pp. 21-73 (1985). Regeneration can also be obtained from plant callus, explants, organs, or parts thereof Such regeneration techniques are described generally in Klee et al., 1987, Ann. Rev. Plant Phys., 38:467-486.

### Functional Genomics Assays

Engineered Zf proteins also have use for assays to determine the phenotypic consequences and function of gene expression. Recent advances in analytical techniques, coupled with focused mass sequencing efforts have created the opportunity to identify and characterize many more molecular targets than were previously available. This new information about genes and their functions will improve basic biological understanding and present many new targets for therapeutic intervention. In some cases analytical tools have not kept pace with the generation of new data. An example is provided by recent advances in the measurement of global differential gene expression. These methods, typified by gene expression microarrays, differential cDNA cloning frequencies, subtractive hybridization and differential display methods, can very rapidly identify genes that are up or down-regulated in different tissues or in response to specific stimuli. Increasingly, such methods are being used to explore biological processes such as, transformation, tumor progression, the inflammatory response, neurological disorders etc. Many differentially expressed genes correlate with a given physiological phenomenon, but demonstrating a causative relationship between an individual differentially expressed gene and the phenomenon is labor intensive. Until now, simple methods for assigning function to differentially expressed genes have not kept pace with the ability to monitor differential gene expression.

The engineered Zf proteins of the present invention can be used to rapidly analyze the function of a differentially expressed gene. Engineered Zf proteins can be readily used to up or down-regulate or knockout any endogenous target gene, or to knock in an endogenous or endogenous gene. Very little sequence information is required to create a gene-specific DNA binding domain. This makes the engineered Zf technology ideal for analysis of long lists of poorly characterized differentially expressed genes. One can simply build a zinc finger-based DNA binding domain for each candidate gene, create chimeric up and down-regulating artificial transcription factors and test the consequence of up or down-regulation on the phenotype under study (e.g., transformation or response to a cytokine) by switching the candidate genes on or off one at a time in a model system.

Additionally, greater experimental control can be imparted by engineered Zf proteins than can be achieved by more conventional methods. This is because the production and/or function of engineered Zf proteins, like other Zf proteins, can be placed under small molecule control. Examples of this approach are provided by the Tet-On system, the ecdysone-regulated system and a system incorporating a chimeric factor including a mutant progesterone receptor. These systems are all capable of indirectly imparting small molecule control on any endogenous gene of interest or any transgene by placing the function and/or expression of a engineered Zf protein under small molecule control.

### Transgenic Animals

A further application of engineered Zf proteins is manipulating gene expression in animal models. As with cell lines, the introduction of a heterologous gene into or knockout of an endogenous in a transgenic animal, such as a transgenic mouse or zebrafish, is a fairly straightforward process. Thus, transgenic or transient expression of an engineered Zf protein in an animal can be readily performed.

By transgenically or transiently expressing a suitable engineered Zf protein fused to an activation domain, a target gene of interest can be over-expressed. Similarly, by transgenically or transiently expressing a suitable engineered Zf protein fused to a repressor or silencer domain, the expression of a target gene of interest can be down-regulated, or even switched off to create “functional knockout”. Knockin or knockout mutations by insertion or deletion of a target gene of interest can be prepared using zinc finger nucleases.

Two common issues often prevent the successful application of the standard transgenic and knockout technology; embryonic lethality and developmental compensation. Embryonic lethality results when the gene plays an essential role in development. Developmental compensation is the substitution of a related gene product for the gene product being knocked out, and often results in a lack of a phenotype in a knockout mouse when the ablation of that gene's function would otherwise cause a physiological change.

Expression of transgenic engineered Zf proteins can be temporally controlled, for example using small molecule regulated systems as described in the previous section. Thus, by switching on expression of an engineered Zf protein at a desired stage in development, a gene can be over-expressed or “functionally knocked-out” in the adult (or at a late stage in development), thus avoiding the problems of embryonic lethality and developmental compensation.

The present invention is illustrated by the following examples, which are not intended to be limiting in any way.

### EXAMPLES

### Example 1

### Creation of a Context-Dependent Assembly (CoDA) Zinc Finger Archive

To demonstrate the applicability of the context-dependent assembly (CoDA) methods for a broad range of potential target sites, a large archive was engineered consisting of 319 F1 and 344 F3 units (shown in FIGS. 3 and 4) that were identified as functioning well when positioned adjacent to one of 18 fixed F2 units in various three-finger arrays. All of the zinc finger units in the archive share the C2H2 motif Cys-(X)₂₋₄-Cys-(X)₁₂-His-(X)₃₋₅-His (SEQ ID NO:840). The F1, F2, and F3 units in the archive share a common sequence (FQCRICMRNFS; SEQ ID NO:841) amino-terminal to the recognition helix. The sequence of the F1, F2, and F3 unit carboxy-terminal to the recognition helices are HTRTH (SEQ ID NO:842), HLRTH (SEQ ID NO:843), and HLKTH (SEQ ID NO:844), respectively.

To identify the “fixed” F2 fingers for various three base pair target subsites, the amino acid sequences of F2s from a collection of three finger arrays previously identified from selections performed for over 130 different nine base pair sites (Maeder et al., 2008, Mol. Cell, 31: 294-301; Foley et al., 2009, PLoS ONE, 4:e4348; Zhang et al., 2010, Proc. Natl. Acad. Sci. USA, 107:12028-33; Townsend et al., 2009, Nature, 459:442-445) were analyzed. From this analysis, F2 units for 18 different 3 base pair subsites that occurred in at least two or more different contexts were identified. The F1 and F3 units found adjacent to these F2 units were also chosen as units because they had been selected to work well together. To obtain additional F1 and F3 units for other 3 base pair subsites, a series of selections were performed in which combinatorial three-finger array libraries composed of a fixed F2 unit and randomized F1 and F3 fingers were interrogated for binding to specific 9 base pair target sequences. From these selections the amino acid sequences of three-finger arrays that activated transcription three-fold or more in the bacterial two-hybrid (B2H) reporter assay were analyzed to identify additional F1 and F3 finger units that worked well when positioned adjacent to a specific fixed F2 unit. For selections that yielded multiple three-finger array clones, F1 and F3 finger units were chosen that occurred the most frequently in multiple distinct arrays and that were found in three-finger arrays that gave the highest fold-activation in the B2H reporter assay. Selections were performed essentially as described (Maeder et al., 2008, Mol. Cell, 31:294-301; Maeder et al., 2009, Nat. Protoc., 4:1471-1501) but with the modification that a beta-lactamase antibiotic resistance gene was used for selection instead of the HIS3 gene.

### Example 2

### Assembly and Testing of Zinc Finger Arrays

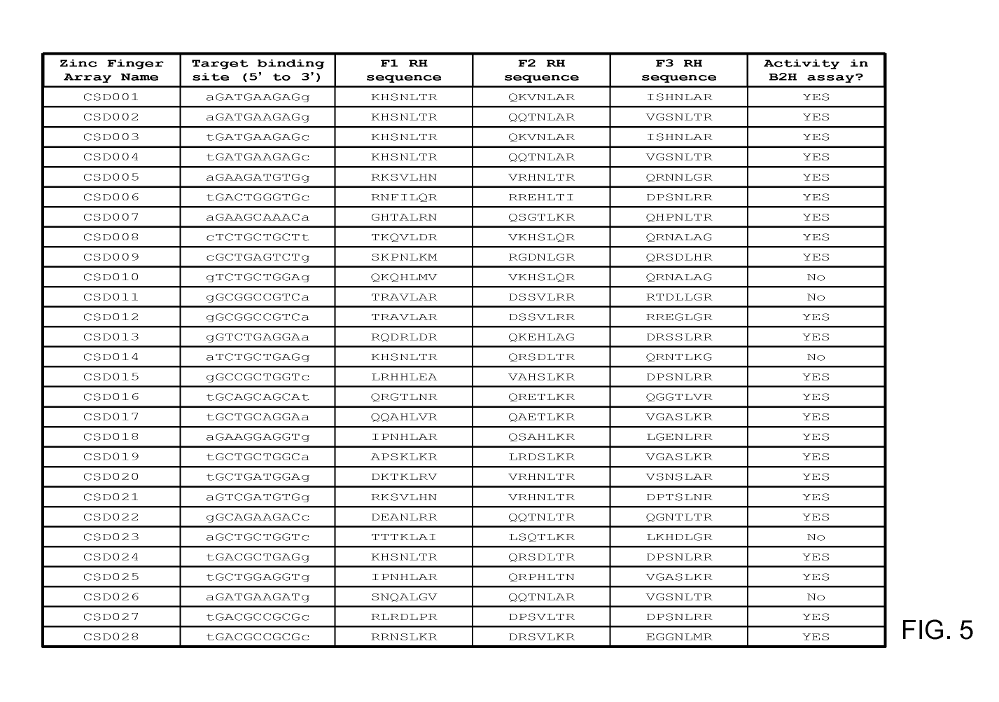

As a pilot experiment, CoDA was used to assemble 26 three-finger arrays each targeted to a specific 9 base pair DNA site (FIG. 5). The DNA-binding activities of these CoDA arrays were tested using a bacterial two-hybrid (B2H) reporter assay. This assay has been shown to identify zinc finger arrays that can bind to their target DNA sites with high affinity and specificity. As summarized in FIG. 5, 21 of the 26 three-finger arrays assembled by CoDA bound to their target site as judged by the B2H assay.

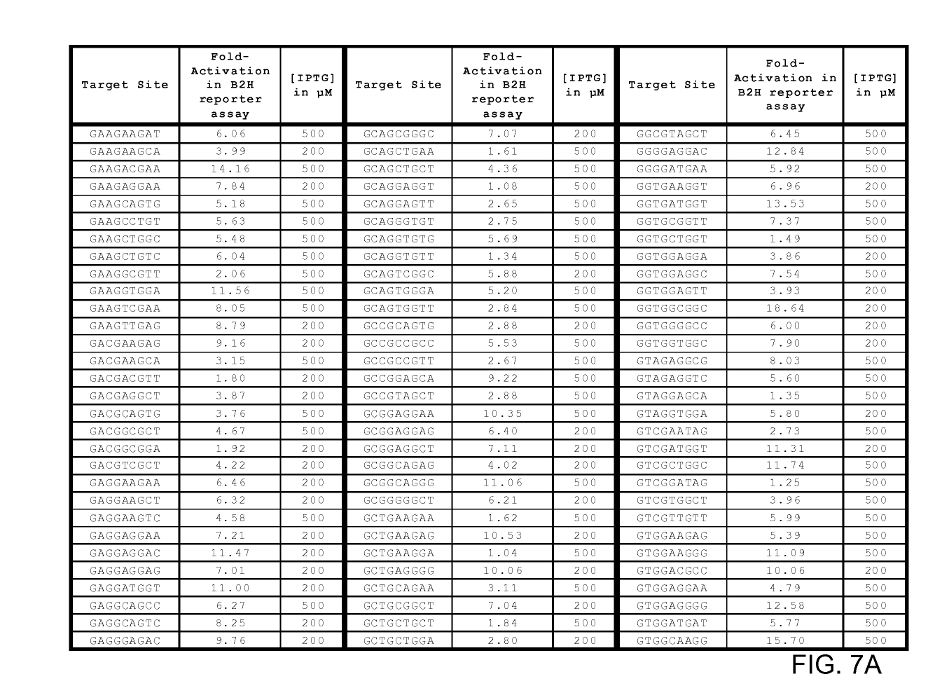

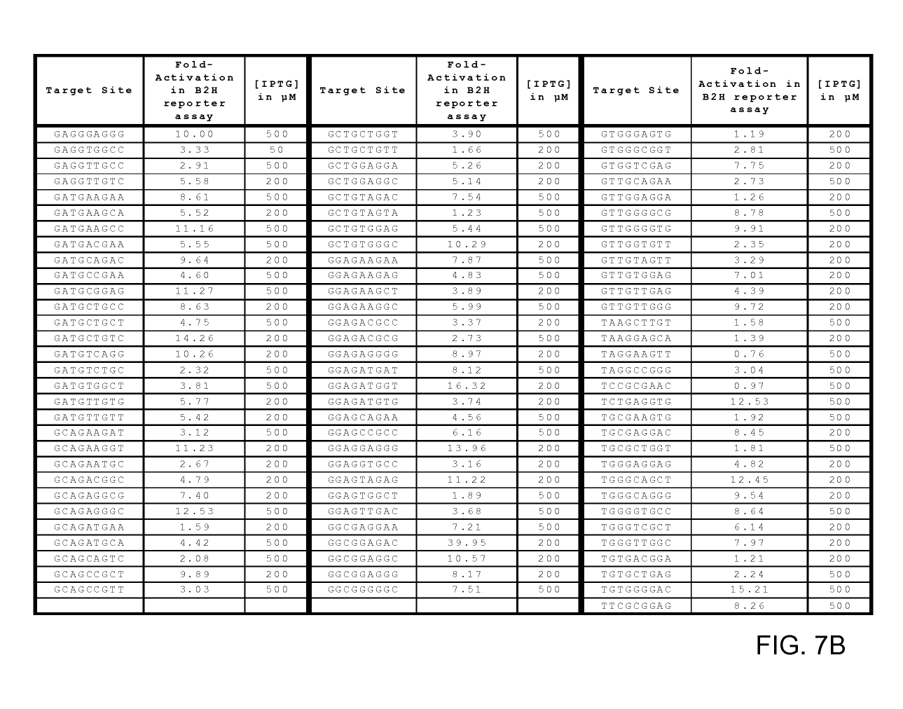

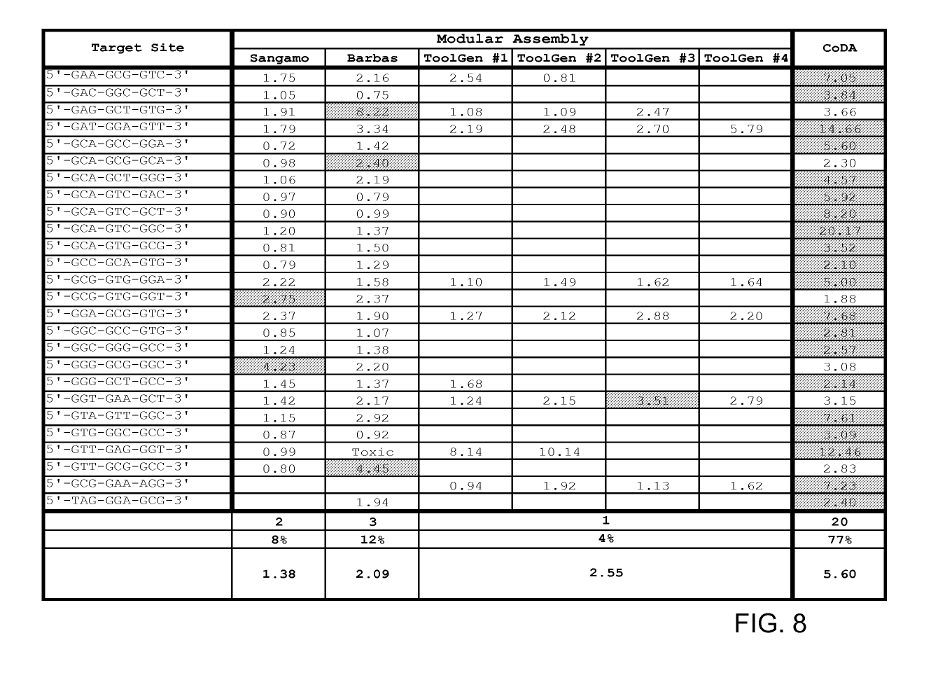

To further test the CoDA approach and the archive of zinc finger units, the method were used to assemble 181 different three-finger arrays and each was experimentally evaluated for its ability to bind its cognate DNA target site using the B2H reporter assay. (The 181 different 9 base pair DNA sites targeted in this experiment are shown in FIG. 7 and are composed of varying numbers of all four nucleotides.) To assemble the zinc finger arrays, DNA fragments encoding a F1-F2 cassette or a F3 cassette were PCR amplified from plasmids using primer pair OK1424/0K1427 or OK1428/0K1429, respectively. (Primer sequences are shown in Table 1 below.) The resulting PCR products were digested with DpnI to degrade template plasmid DNA and cleaned up using a QIAGEN PCR purification kit. The cassettes were then fused together and amplified in a single PCR step using primer pair OK1430/0K1432. PCR product encoding a three-finger array was then cleaned up using a QIAGEN PCR purification kit, treated with Pfu polymerase in the presence of dTTP nucleotide to create overhangs, phosphorylated with T4 polynucleotide kinase, and ligated to a B2H expression plasmid (pMG414) in which the zinc finger array is expressed as a fusion to a fragment of the yeast Gal11P protein (Maeder et al., 2009, Nat. Protoc., 4:1471-1501). All plasmids were sequence-verified using primer OK61.

TABLE 1
Primer sequences

| | | SEQ |
|---|---|---|
| Primer | | ID |
| Name | Primer Sequence | NO |
| OK1424 | 5′-GAGCGCCCCTTCCAGTGTCGC-3′ | 833 |
| OK1427 | 5′-TCGGCATTGGAATGGCTTCTCG-3′ | 834 |
| OK1428 | 5′-GCCATTCCAATGCCGAATATGCA-3′ | 835 |
| OK1429 | 5′-CCCTCAGGTGGGTTTTTAGGTG-3′ | 836 |
| OK1430 | 5′-GGGGAGCGCCCCTTCCAGTGTCGC-3′ | 837 |
| OK1432 | 5′-GTGCAGAGGATCCCCTCAGGTGGGTTTTTAGGTG-3′ | 838 |
| OK61 | 5′-GGGTAGTACGATGACGGAACCTGTC-3′ | 839 |

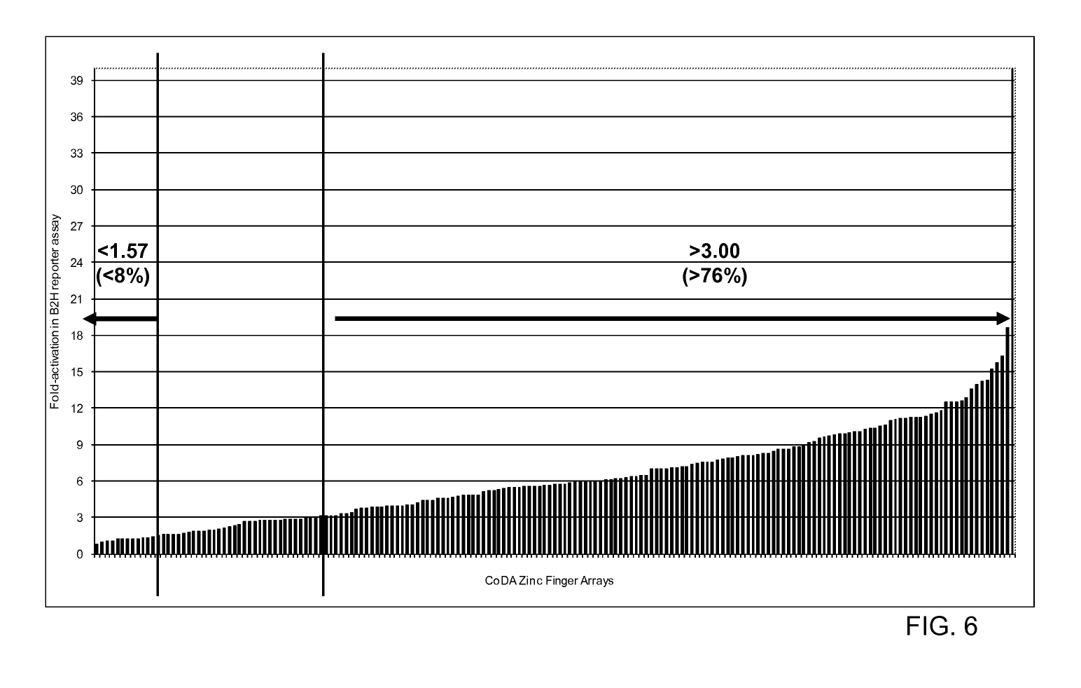

Previous work has shown that three-finger arrays that fail to activate transcription by more than 1.57-fold in the B2H reporter assay are inactive as zinc-finger nucleases (ZFNs) in mammalian cells (Ramirez et al., 2008, Nat. Methods, 5:374-375). Of the 181 three-finger arrays made, 168 of them (>92%) activated transcription by >1.57-fold (FIGS. 6 and 7). In addition, three-finger arrays that activate transcription by more than three-fold in the B2H reporter assay have a high probability of functioning efficiently as ZFNs in zebrafish (Foley et al., 2009, PLoS ONE 4:e4348), plant (Zhang et al., 2010, Proc. Natl. Acad. Sci. USA, 107:12028-33; Townsend et al., Nature, 459:442-445), and human cells (Maeder et al., 2008, Mol. Cell, 31:294-301; Cornu et al., 2008, Mol. Ther., 16:352-358; Pruett-Miller et al., 2008, Mol. Ther., 16:707-717; Zou et al., 2009, Cell Stem Cell, 5:97-110). Strikingly, 139 of 181 the arrays described herein (>76%) activated transcription more than three-fold in the B2H reporter assay (FIGS. 6 and 7A-B). These frequencies of predicted failure and success (as predicted by the B2H reporter assay) are comparable to those previously observed with three-finger arrays made using selection methods (Maeder et al., 2008, Mol. Cell, 31: 294-301; Foley et al., 2009, PLoS ONE, 4:e4348) (Table 2). Furthermore, because very few arrays (<25%) scored as inactive in the B2H reporter assay, these results suggest that this step can be skipped and that assembled CoDA ZFNs can be tested directly in the final desired cell type of interest.

TABLE 2
Comparison of selection and CoDA methods

Fold-activation in B2H
reporter assay

| Method | <1.57 | >3.00 |
|---|---|---|
| Selection | 5.3% | 86.8% |
| CoDA | 7.2% | 76.8% |

### Example 3

### Comparison of CoDA and Modular Assembly Methods

The efficacy of CoDA was directly compared with that of modular assembly by using both approaches to construct three-finger arrays for 26 different nine base pair sites and testing these proteins for DNA-binding activity in the B2H reporter assay. The DNA sites used for this experiment (FIG. 8) were chosen from among 104 sites that had been previously tested to assess the efficacy of modular assembly (Ramirez et al., 2008, Nat. Methods, 5:374-375). Nearly all of these sites (24 out of 26) matched the consensus sequence 5′ GNNGNNGNN3′, a category of target sites for which modular assembly showed the highest success rates in an earlier report (Ramirez et al., 2008, Nat. Methods, 5:374-375). In addition, it is important to note that although only one CoDA finger array was made and tested for each of the 26 target sites, multiple modularly assembled arrays (two to six arrays) were made and tested for nearly all (25 of the 26) sites (FIG. 8). Multiple modularly assembled arrays can be made using three published module archives from the Sangamo (Liu et al., 2002, J. Biol. Chem., 277:3850-56), Barbas (Mandell et al., 2006, Nucleic Acids Res., 34:W516-523), and Toolgen (Bae et al., 2003, Nat. Biotechnol., 21:275-280) groups. Despite this advantage in numbers of proteins per target site, the results demonstrated that CoDA yielded the zinc finger array with the highest or second highest B2H assay activity for 25 of the 26 target sties, and the highest B2H assay activity for 20 of the 26 target sites (FIG. 8). Furthermore, the mean B2H fold-activation of all CoDA proteins tested (5.59-fold) was higher than those made using the three different modular assembly sets (1.43-, 2.11-, and 2.53-fold for the Sangamo, Barbas, and Toolgen modules, respectively; FIG. 8).

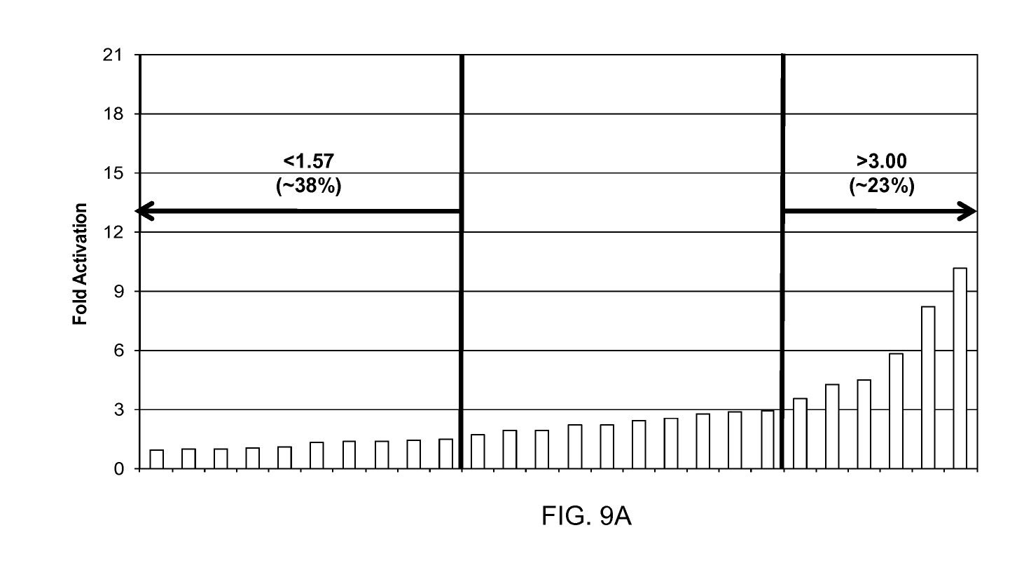

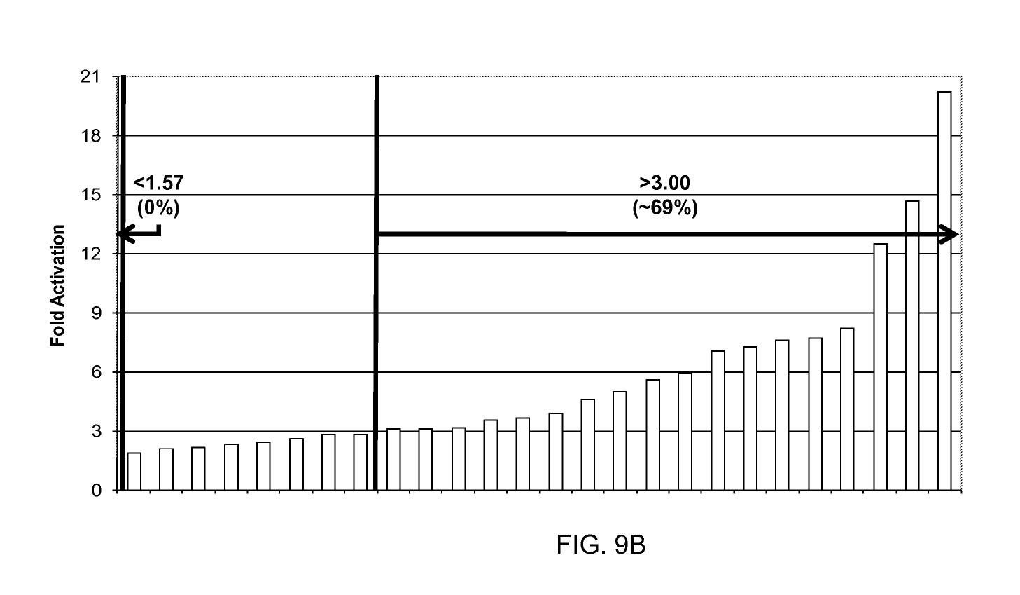

To compare success and failure rates of CoDA and modular assembly, fold-activation values in the B2H reporter assay of the most active protein made by each of the two methods for the 26 target DNA sites were examined. Of these proteins, ˜38% of the modular assembled arrays activated transcription by 1.57-fold or less in the B2H (FIG. 9A) compared with 0% of the CoDA arrays (FIG. 9B). Furthermore, only ˜23% of the modularly assembled arrays activated transcription by three-fold or more in the B2H assay (FIG. 9A) compared with ˜69% of the CoDA arrays (FIG. 9B). Taken together, these results clearly demonstrate that CoDA consistently outperforms modular assembly in direct comparisons. Furthermore, the differences in failure and success rates between the two approaches becomes even more significant when one considers that two functional arrays must be engineered to create dimers of ZFNs required for genome modification.

### Example 4

### Use of CoDA to Engineer Zinc Finger Nucleases

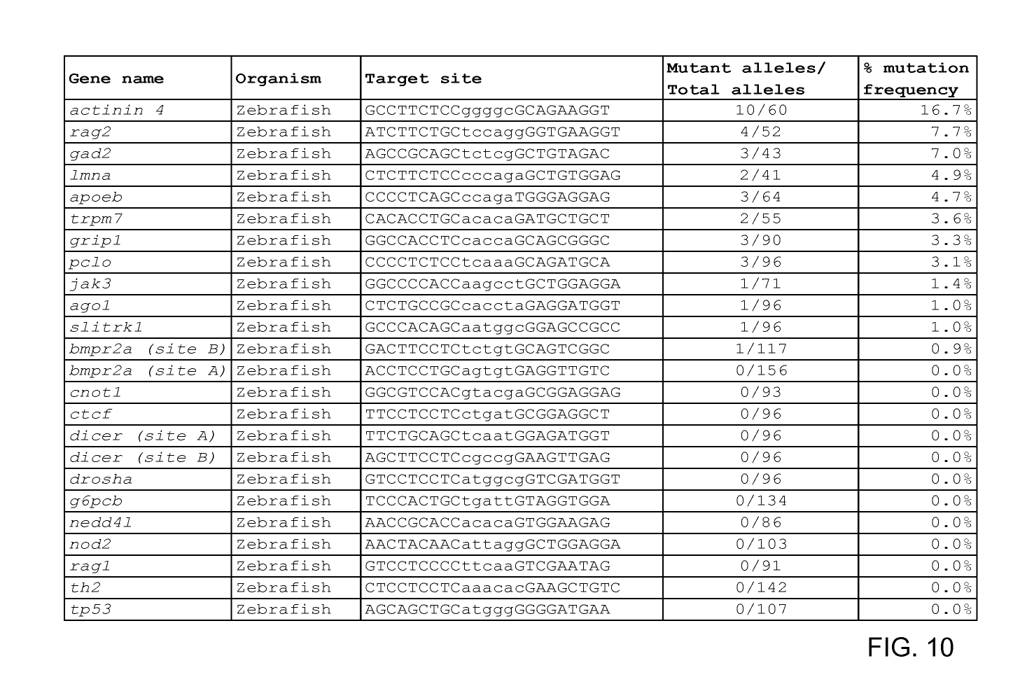

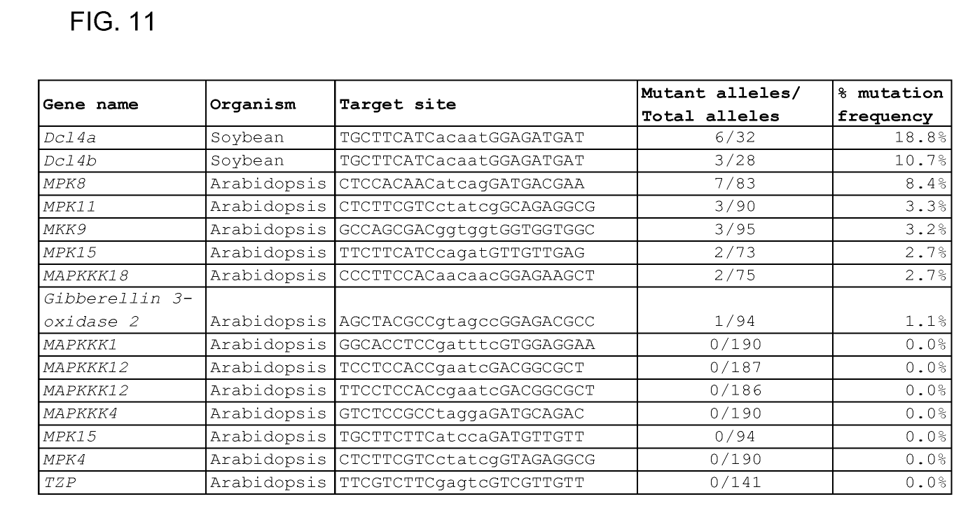

To further test the speed and efficacy of CoDA, method was used to make ZFNs for a large number of endogenous gene targets in zebrafish and plants. These organisms were chosen for testing of CoDA ZFNs because methods for using ZFNs are well established for both, and because demand for ZFNs from these communities is considerable due to the unique targeted mutation capability conferred by the technology. Using CoDA zinc finger arrays that activated transcription at least three-fold in the B2H reporter assay, ZFN pairs were constructed for 24 gene targets in zebrafish, 13 gene targets in Arabidopsis thaliana, and one target present in two duplicated genes in soybean (FIGS. 10 and 11).

For zebrafish, ZFN-induced mutations were assessed in somatic cells from normal appearing embryos, and CoDA ZFNs were able to induce targeted insertion or deletion mutations with high efficiencies for 12 out of 24 zebrafish target sites tested (FIG. 10). The CoDA ZFN-induced mutation frequencies observed in these somatic cell experiments (0.9% to 16.7%) are comparable to those from previous experiments in which founders capable of transmitting mutations through the germline were readily identified (Foley et al., 2009, PLoS ONE, 4:e4348).

For plants, it was tested whether CoDA ZFNs could induce mutations in Arabidopsis and soybean genes. CoDA ZFNs induced insertion or deletion mutations with high frequencies (1.1% to 8.4%) in six of 13 gene targets in Arabidopsis (FIG. 11). These frequencies of mutagenesis (as measured by number of mutated alleles) are comparable to those previously observed with ZFNs made by selection methods (Zhang et al., 2010, Proc. Natl. Acad. Sci. USA, 107:12028-33). In addition, a pair of ZFNs made by CoDA very efficiently introduced mutations into a target site present in two duplicated soybean genes (frequencies of 18.8% and 10.7% in transformed root tissue; FIG. 11). No comparisons to prior experiments could be made for the soybean experiments because, to our knowledge, these are the first examples of ZFN-targeted mutations in endogenous soybean genes.

The overall success rate for obtaining mutations with CoDA ZFNs on a per target basis was 50% (19 out of 38 target sites) in zebrafish and plants. A comparable historical success rate of ˜67% with selected ZFNs has been observed in zebrafish, plants, and human cells (16 out of 24 target sites; Maeder et al., 2008, Mol. Cell, 31:294-301; Foley et al., 2009, PLoS ONE, 4:e4348; Zhang et al., 2010, Proc. Natl. Acad. Sci. USA, 107:12028-33; Townsend et al., 2009, Nature, 459:442-445; Zou et al., 2009, Cell Stem Cell, 5:97-110). The simplicity and high success rate of the CoDA method enabled the mutation in this disclosure of more endogenous zebrafish and plant genes (12 and 8, respectively) than the cumulative total of all previously published reports combined (10 zebrafish genes [Doyon et al., 2008, Nat. Biotechnol., 26:702-708; Foley et al., 2009, PLoS ONE 4:e4348; Meng et al., 2008, Nat. Biotechnol., 26:695-701; Siekmann et al., 2009, Genes Dev., 23:2272-77; Cifuentes et al., 2010, Science, 328:1694-98] and 7 plant genes [Zhang et al., 2010, Proc. Natl. Acad. Sci. USA, 107:12028-33; Townsend et al., 2009, Nature, 459:442-445; Shukla et al., 2009, Nature, 459:437-441; Cai et al., 2009, Plant Mol. Biol., 69:699-709; Osakabe et al., 2010, Proc. Natl. Acad. Sci. USA, 107:12034-39]).

Although it is unclear why both CoDA and selected ZFNs fail to induce mutations at approximately half of the sites targeted, chromatin state or DNA methylation of the site (rather than DNA binding activities of the ZFNs) may be responsible, since the ZFNs appear to possess sequence-specific DNA-binding activities for their target sites as judged by the B2H reporter assay results. Regardless of the precise mechanism, users of CoDA can make ZFNs for at least two target sites per gene of interest to increase the likelihood that at least one pair will successfully introduce mutations. Further, although the tests described herein are limited to zebrafish and plants, CoDA ZFNs can also work in mammalian cells because zinc finger arrays that activate transcription three-fold or more in the B2H reporter assay have been shown to function efficiently as ZFNs in human cells (Maeder et al., 2008, Mol. Cell, 31:294-301; Cornu et al., 2008, Mol. Ther., 16:352-358; Pruett-Miller et al., 2008, Mol. Ther., 16:707-717; Zou et al., 2009, Cell Stem Cell, 5:97-110).

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A method of designing a multi-zinc-finger polypeptide sequence predicted to bind to a nucleic acid sequence of interest comprising at least three subsites, the method comprising:
a) providing a nucleotide sequence of interest comprising first, second, and third consecutive subsites, wherein each of the first and third subsites are adjacent to the second subsite;
b) identifying first and second adjacent zinc finger polypeptide sequences previously shown to bind to the first and second subsites in the context of a multi-zinc finger polypeptide;
c) identifying a third zinc finger polypeptide sequence shown to bind to a third subsite adjacent to the second subsite when present in the context of a multi-zinc finger polypeptide adjacent to the second zinc finger polypeptide sequence; and
d) combining the first, second, and third zinc finger polypeptide sequences in linear order, thereby designing a multi-zinc finger polypeptide sequence predicted to bind to the sequence of interest.

2. The method of claim 1, further comprising producing a polynucleotide comprising a sequence that encodes a polypeptide comprising the multi-zinc-finger polypeptide.

3. The method of claim 1, further comprising producing a polypeptide comprising the multi-zinc-finger polypeptide sequence.

4. The method of claim 1, wherein the first subsite is located 5′ to the second subsite.

5. The method of claim 1, wherein the first subsite is located 3′ to the second subsite.

6. The method of claim 1, wherein the second zinc finger sequence comprises a sequence selected from SEQ ID NOs: 1-18.

7. The method of claim 6, wherein the first zinc finger sequence comprises a sequence selected from SEQ ID NOs: 19-337.

8. The method of claim 6, wherein the third zinc finger sequence comprises a sequence selected from SEQ ID NOs: 338-681.

9. A polypeptide produced by the method of claim 3.

10. The polypeptide of claim 9, wherein the polypeptide comprises one or more functional domains.

11. The polypeptide of claim 10, wherein the functional domain is selected from the group comprising transcriptional activation domain, transcriptional repressor domain, transcriptional silencing domain, acetylase domain, de-acetylase domain, methylation domain, de-methylation domain, kinase domain, phosphatase domain, dimerization domain, multimerization domain, nuclear localization domain, nuclease domain, endonuclease domain, resolvase domain and integrase domain.

12. The polypeptide of claim 9, wherein the functional domain is an endonuclease domain.

13. A method of regulating the expression of a gene comprising contacting a polypeptide according to claim 10 with a sequence of interest in the gene to form a binding complex, such that expression of the gene is regulated.

14. A method of altering the structure of a gene comprising contacting a zinc finger polypeptide according to claim 10 with a sequence of interest within the gene to form a binding complex, such that the structure of the gene is altered.

15. A method of cleaving a sequence of interest comprising contacting a zinc finger polypeptide according to claim 10 with the sequence of interest to form a binding complex, such that the sequence of interest is cleaved.

16. A set of multi-zinc finger array sequences, wherein each array comprises at least first, second, and third adjacent zinc fingers, wherein the sequence of the second zinc finger is identical for each entry in the database, and wherein the database comprises at least ten entries.

17. A method of creating a set of multi-zinc-finger array sequences, the method comprising:
providing a parent zinc finger polypeptide comprising at least first, second, and third adjacent zinc fingers, wherein the zinc finger polypeptide binds to a known parental target sequence comprising at least first, second, and third adjacent subsites;
producing a library of zinc finger polypeptides based on the parent zinc finger polypeptide sequence, wherein each member of the library comprises the parental second zinc finger sequence and the sequence of either or both of the first and third fingers are varied; and
selecting members of the library of zinc finger polypeptides that bind to one or more target sequences comprising the parental second subsite and either or both of a non-parental first and third subsite, thereby providing a set of multi-zinc-finger array sequences with common second finger sequences.

18. The method of claim 17, wherein the library is expressed in vitro.

19. The method of claim 17, wherein the library is expressed in an expression system selected from the group consisting of eukaryotic, prokaryotic and viral expression systems.

20. The method of claim 19, wherein the library is expressed in bacteria.
